(19) United States
(12) Patent Application Publication
(10) Pub. No.: US 2021/0395312 A1
(43) Pub. Date: Dec. 23, 2021

(54) QUANTUM CONFINED PEPTIDE ASSEMBLIES AND USES THEREOF

(71) Applicant: Ramot at Tel-Aviv University Ltd., Tel-Aviv (IL)

(72) Inventors: Ehud GAZIT, Tel-Aviv (IL); Kai TAO, Tel-Aviv (IL); Yu CHEN, Tel-Aviv (IL); Sharon GILEAD, Tel-Aviv (IL); Sigal LAZAR, Tel-Aviv (IL); Pandeeswar MAKAM, Tel-Aviv (IL)

(73) Assignee: Ramot at Tel-Aviv University Ltd., Tel-Aviv (IL)

(21) Appl. No.: 17/467,297

(22) Filed: Sep. 6, 2021

Related U.S. Application Data
(63) Continuation of application No. PCT/IL2020/050265, filed on Mar. 6, 2020.
(60) Provisional application No. 62/814,946, filed on Mar. 7, 2019.

(51) Int. Cl. C07K 7/64

(52) U.S. Cl. CPC C07K 7/64; B82Y 30/00

(57) ABSTRACT

Self-assembled structures formed of a plurality of cyclic peptides which are in association with metal ions is provided. The cyclic peptides are each of from 2 to 6 amino acid residues, and two or more of the amino acid residues are each independently an aromatic amino acid residue. The self-assembled structures exhibit photoluminescence and can be used or incorporated in light emitting systems.

### RELATED APPLICATIONS

This application is a Continuation of PCT Patent Application No. PCT/IL2020/050265, having the international filing date of Mar. 6, 2020 which claims the benefit of priority under 35 USC § 119(e) of U.S. Provisional Patent Application No. 62/814,946 filed on Mar. 7, 2019. The contents of the above applications are all incorporated by reference as if fully set forth herein in their entirety.

The project leading to this application has received funding from the European Research Council (ERC) under the European Union's Horizon 2020 research and innovation programme (grant agreement No 694426).

### FIELD AND BACKGROUND OF THE INVENTION

The present invention, in some embodiments thereof, relates to peptide-based materials and, more particularly, but not exclusively, to quantum confined peptide assemblies which feature tunable photoluminescence and to uses thereof as, for example, photoactive materials and carriers for drug delivery.

Quantum confinement describes a change of electronic and optical properties of when the material sampled is of sufficiently small size—typically 10 nanometers or less. The bandgap increases as the size of the nanostructure decreases. More specifically, quantum confined materials can exhibit a change in emission and/or absorption spectrum of a quantum well, dot, disks or other quantum systems or objects upon application of an external electric field.

Quantum confined (QC) materials, such as quantum dots (QDs), have been widely employed for imaging due to their remarkable photoluminescent properties. However, currently known inorganic QC constituents, such as cadmium-based QDs, are intrinsically cytotoxic, thus limiting their applications. Although organic fluorescent dyes allow to overcome the potential cytotoxicity to some extent, several concerns, including weak sustainability and photobleaching, narrow color spectrum, and in some cases complicated synthesis procedures, still practically impede their utilization.

Bioinspired supramolecular structures, especially peptide self-assemblies, were found to show extensive quantum confinement effects and demonstrate remarkable photoluminescence that could offer some benefits over the state-of-the-art counterparts.

However, the majority of intrinsically fluorescent peptides have low quantum yields and photostability, which severely hinders their practical applications, and specifically limits their potential as eco-friendly materials for optoelectronic devices and efficient bioimaging probes. The quest for eco-friendly, organic, tunable and flexible QC alternatives with improved and stable photoluminescence is continuously ongoing.

Accumulating studies demonstrate that aromatic linear-dipeptides, with the representative model of diphenylalanine (FF), can self-assemble into nanostructures with remarkable physiochemical features, such as optic, electrical, piezoelectric (including ferroelectric and pyroelectric) properties. It has been shown that the supramolecular morphologies and properties can be easily modified by amino acids substitutions, covalent conjugation or co-assembly with external moieties. For example, upon substitution of one F with tryptophan (W), self-assembling FW nanostructures present a smaller bandgap of 2.25 eV, compared to 3.25 eV of FF nanotubes, thus showing improved conductive and photoluminescent properties. See, for example, Tao et al. Science. 2017 November 17; 358(6365): doi:10.1126/science.aam9756.

Recent studies revealed that cyclo-dipeptides with backbones of 2,5-diketopiperazine configurations, derived from dehydration condensation of linear dipeptides, self-assemble into photoluminescent nanostructures different from their linear counterparts [Lee, J. S. et al. Angew. Chem. Int. Ed. 50, 1164-1167 (2011); Yan et al. Angew. Chem. Int. Ed. 50, 11186-11191 (2011); Manchineella, S. & Govindaraju, T. ChemPlusChem 82, 88-106 (2016); and Amdursky, N. et al. Biomacromolecules 12, 1349-1354 (2011)].

Cyclic-peptides derived from amino acid residues carrying complexing side chain substituents, such as imidazole, carboxylate or thioether groups, can be used as models to mimic the coordination of metal ions in enzymes. [Ma et al. J. Am. Chem. Soc. 2014, 136 (51), 17734-17737; Clark et al. J. Am. Chem. Soc. 1998, 120 (4), 651-656; Bellezza et al. Trends in Molecular Medicine 2014, 20 (10), 551-558; Anderson et al. Coordination Chemistry Reviews 2017, 349, 102-128; Zou et al. Chemical Society Reviews 2015, 44 (15), 5200-5219; and Mannini et al. ACS Chemical Neuroscience 2018, 9 (12), 2959-2971].

Cyclic-dipeptides are highly tunable due to hydrogen bonding capabilities of the skeleton and other noncovalent interactions, that can be used to engineer artificial multifunctional scaffolds [Montenegro et al. Accounts of Chemical Research 2013, 46 (12), 2955-2965; Mantion et al. J. Am. Chem. Soc. 2008, 130 (8), 2517-2526].

Additional background art includes WO 2010/038228; Gazit, E. Peptide nanostructures: aromatic dipeptides light up. Nature Nanotechnol. 11, 309-310 (2016); Tao, K. et al. Nature Commun. 9, 3217 (2018); Tao, K. Peptide Semiconductor Times Are Coming. Go(dot)nature(dot)com/2MgoxSF; Kai Tao, Ehud Gazit. Aromatic peptide assemblies as bio-inspired supramolecular semiconductors. Peptide Self-Assembly: Biology, Chemistry, Materials and Engineering, Beijing, China, August 2018 (Poster); Kai Tao, Ehud Gazit. Aromatic cyclo-dipeptide self-assemblies with quantum confined photoluminescence from visible to near-infrared ranges. The 5^{th} BioE12018 International Winterschool on Bioelectronics, Kirchberg in Tirol, Austria, March 2018 (Poster); Tao et al. Science 358, eaam9756 (2017); Tao et al., Mater Today (Kidlington) Author manuscript; available in PMC 2019 Nov. 12; Yuan et al., Research (Wash D.C.) 2019; 2019:9025939 doi: 10.34133/2019/9025939; and Tao et al., Adv. Funct. Mater. 2020, 1909614, all of which are incorporated by reference as of fully set forth herein.

### SUMMARY OF THE INVENTION

According to an aspect of some embodiments of the present invention there is provided a light emitting system, comprising a self-assembled structure formed of a plurality of cyclic peptides, at least a portion of the plurality of cyclic peptides being in association with metal ions, wherein each cyclic peptide in the plurality of cyclic peptides independently comprises from 2 to 6 amino acid residues, wherein at least two of the amino acid residues are each independently an aromatic amino acid residue, and wherein at least one of the aromatic amino acid residues comprises an imidazole in its side chain, wherein the self-assembled plurality of cyclic peptides exhibits photoluminescence.

According to some of any of the embodiments described herein, in at least a portion, or all, of the plurality of cyclic peptides, at least one amino acid residue is an L-amino acid residue and at least one amino acid residue is a D-amino acid residue.

According to some of any of the embodiments described herein, in at least a portion, or all, of the plurality of cyclic peptides, each cyclic peptide comprises at least two aromatic amino acid residues, each independently comprising the imidazole.

According to some of any of the embodiments described herein, each cyclic peptide in at least a portion, or all, of the plurality of cyclic peptides is a cyclic dipeptide.

According to some of any of the embodiments described herein, in at least a portion, or all, of the plurality of cyclic peptides, each cyclic peptide is a cyclic homodipeptides which comprises two amino acid residues, each independently comprising the imidazole.

According to some of any of the embodiments described herein, the cyclic homodipeptide comprises one L-amino acid residue and one D-amino acid residue.

According to some of any of the embodiments described herein, each of the amino acid residues is a histidine residue.

According to an aspect of some embodiments of the present invention there is provided a light emitting system comprising a self-assembled structure formed of a plurality of cyclic peptides, at least a portion of the cyclic peptides being in association with metal ions, wherein each cyclic peptide in the plurality of cyclic peptides independently comprises from 2 to 6 amino acid residues, wherein at least two of the amino acid residues are each independently an aromatic amino acid residue, wherein the self-assembled plurality of cyclic peptides exhibits photoluminescence.

According to some of any of the embodiments described herein, in at least a portion, or all, of the plurality of cyclic peptides, each cyclic peptide is a cyclic dipeptide.

According to some of any of the embodiments described herein, in at least a portion, or all, of the plurality of cyclic peptides, each cyclic peptide comprises at least one aromatic amino acid that comprises an imidazole in its side-chain.

According to some of any of the embodiments described herein, the cyclic dipeptide is a cyclic heterodipeptide that does not comprise a tryptophan residue.

According to some of any of the embodiments described herein, in at least a portion, or all, of the plurality of cyclic peptides, each amino acid residue has the same chirality.

According to some of any of the embodiments described herein, in at least a portion, or all, of the plurality of cyclic peptides, each amino acid residue is an L-amino acid residue.

According to some of any of the embodiments described herein, in at least a portion, or all, of the plurality of cyclic peptides, at least one amino acid residue is an L-amino acid residue and at least one amino acid residue is a D-amino acid residue.

According to some of any of the embodiments described herein, in at least a portion, or all, of the plurality of cyclic peptides, each cyclic peptide is a cyclic homodipeptide.

According to some of any of the embodiments described herein, the cyclic homodipeptide comprises one L-amino acid residue and one D-amino acid residue.

According to some of any of the embodiments described herein, the association with the metal ions modulates at least one property of the photoluminescence of the self-assembled plurality of cyclic peptides.

According to some of any of the embodiments described herein, the photoluminescence property is selected from emission wavelength, excitation wavelength, quantum yield, photoluminescence time, and photoluminescence stability.

According to some of any of the embodiments described herein, the association with the metal ions modulates an emission wavelength of the self-assembled plurality of cyclic peptides.

According to some of any of the embodiments described herein, the association with the metal ions redshifts an emission wavelength of the self-assembled plurality of cyclic peptides.

According to some of any of the embodiments described herein, the light emitting system exhibits, upon excitation, an emission wavelength of at least 400 nm.

According to some of any of the embodiments described herein, the metal ions are multivalent metal ions.

According to some of any of the embodiments described herein, the metal ions form a part of a metal salt.

According to some of any of the embodiments described herein, the metal is selected from zinc, copper, silver, gold, magnesium, manganese, cadmium, other transitions metals, lanthanide metal and actinide metal.

According to some of any of the embodiments described herein, the self-assembled structure has an average size of less than 100 nm at least in one dimension or cross-section.

According to some of any of the embodiments described herein, light emitting system further comprises an excitation system configured to excite the self-assembled structure to emit light.

According to some of any of the embodiments described herein, the light emitting system further comprises a therapeutically active agent in association with the self-assembled structure. Such a system is a drug delivery system that can be used for treating and/or monitoring a medical condition treatable by the therapeutically active agent. In some of these embodiments, the system is capable of releasing the active agent at a targeted diseased site. In some of these embodiments, the system is capable of binding targeted (e.g., diseased) cell membranes and/or of delivering the therapeutically active agent to the cell's nucleus. In some of these embodiments, the system features cell-permeability.

According to an aspect of some embodiments of the present invention there is provided a pharmaceutical composition comprising the light emitting system as described herein in any of the respective embodiments and any combination thereof, and optionally a pharmaceutically acceptable carrier.

According to an aspect of some embodiments of the present invention there is provided a light emitting system as described herein in any of the respective embodiments and any combination thereof or the pharmaceutical composition as described herein, for use in a method of treating a subject having a medical condition treatable by the therapeutically active agent and/or for monitoring the medical condition and/or for monitoring the treating.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Implementation of the method and/or system of embodiments of the invention can involve performing or completing selected tasks manually, automatically, or a combination thereof. Moreover, according to actual instrumentation and equipment of embodiments of the method and/or system of the invention, several selected tasks could be implemented by hardware, by software or by firmware or by a combination thereof using an operating system.

For example, hardware for performing selected tasks according to embodiments of the invention could be implemented as a chip or a circuit. As software, selected tasks according to embodiments of the invention could be implemented as a plurality of software instructions being executed by a computer using any suitable operating system. In an exemplary embodiment of the invention, one or more tasks according to exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

### BRIEF DESCRIPTION OF THE SEVERAL VIEWS OF THE DRAWING(S)

The patent or application file contains at least one drawing executed in color. Copies of this patent or patent application publication with color drawing(s) will be provided by the Office upon request and payment of the necessary fee.

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 presents the 2D chemical structures of exemplary cyclo-dipeptides featuring a diketopiperazine skeleton according to some embodiments of the present invention, cyclo-FW and cyclo-WW.

FIGS. 2A-B present the excitation spectra of cyclo-FW and cyclo-WW monomers (thin light curves, 0.05 mM) and self-assemblies (thick dark curves, 5.0 mM) in MeOH (FIG. 2A); The excitation wavelengths red shifted from 285 nm to about 305 nm after self-assembly, and the UV-Vis absorption spectra of cyclo-FW and cyclo-WW (FIG. 2B) after self-assembly, showing spike-like absorptions at 273 nm, 280 nm and 289 nm, characteristic of the formation of quantum dot (QD) structures.

FIG. 3 is a schematic representation of the process of cyclo-dipeptides self-assembly: the monomers form dimeric QDs, which serve as the building blocks to self-assemble into larger QC architectures.

FIGS. 4A-B present calculated molecular orbital amplitude plots and energy levels of the highest occupied and lowest unoccupied molecular orbitals of cyclo-FW (FIG. 4A) and cyclo-WW (FIG. 4B), showing band gaps of 3.63 eV and 3.56 eV, respectively.

FIG. 5 presents plots showing DLS characterization of cyclo-dipeptides self-assembly (5.0 mM in MeOH). The results demonstrate that both cyclo-dipeptides self-assembled into larger particles several hundred nanometers in size.

FIG. 6 presents the fluorescent emission of cyclo-FW (blue) and cyclo-WW (red) self-assemblies. The insets show the corresponding solutions under UV light (365 nm). Intrinsic fluorescent emissions of the corresponding cyclo-dipeptides were added and marked in black for comparison.

FIG. 7 presents the fluorescent emission of cyclo-WW+Zn(II). The insets show the corresponding solutions under UV light (365 nm). Intrinsic fluorescent emissions of the corresponding cyclo-dipeptides were added and marked in black for comparison.

FIGS. 8A-B present the height measurement of dimers self-assembled by cyclo-WW+Zn(II) in MeOH. FIG. 8A is an AFM micrograph (Scale bar: 500 nm) and FIG. 8B shows the cross-section profile corresponding to the black line in FIG. 8A, demonstrating that the size of the dimers was approximately 3.0 nm.

FIG. 9 presents the DLS analysis of cyclo-WW+Zn(II) in MeOH. The single peak at 2.88 nm demonstrates that only dimers were present in the solution.

FIG. 10 presents the fluorescent emission of cyclo-Fw. The insets show the corresponding solutions under UV light (365 nm). Intrinsic fluorescent emissions of the corresponding cyclo-dipeptides were added and marked in black for comparison.

FIG. 11 presents DLS characterization of the size distributions of cyclo-Fw self-assemblies in MeOH. The results demonstrate that the cyclo-dipeptides self-assembled into larger supramolecular structures, several hundred nanometers in size.

FIGS. 12A-B present the fluorescent emission of cyclo-WW+Cu(II) (FIG. 12A) and cyclo-WW+UV (FIG. 12B). The insets show the corresponding solutions under UV light (365 nm). Intrinsic fluorescent emissions of the corresponding cyclo-dipeptides were added and marked in black for comparison.

FIGS. 13A-B present the DLS characterization of the size distributions of cyclo-WW+Cu(II) (FIG. 13A) and cyclo-WW+UV (FIG. 13B) self-assemblies in MeOH. The results demonstrate that the cyclo-dipeptides self-assembled into larger supramolecular structures, several hundred nanometers in size.

FIG. 14 is a bar graph showing the lifetime statistics of cyclo-WW self-assemblies. Left panel (red): cyclo-WW in MeOH (cyclo-WW: 370/425; +Zn(II): 370/520; +Cu(II): 370/465; +UV: 370/465). Right panel (blue): cyclo-WW+Zn(II) in DMSO (540/610; 670/712; 740/817). Error bars on lifetime measures show standard deviations for three replicates.

FIGS. 15A-F present SEM and AFM images of the cyclo-dipeptides QC self-assemblies in MeOH. FIG. 15A show needle-like cyclo-FW crystals. Scale bar: 40 μm. FIG. 15B show spherical cyclo-WW nanoparticles. Scale bar: 20 μm. FIG. 15C show dimeric QDs of cyclo-WW+Zn(II). Scale bar: 500 nm. FIG. 15D show Nano-flower architectures of cyclo-Fw. Scale bar: 20 μm. FIGS. 15E and 15F show larger spherical nanoparticles of cyclo-WW+Cu(II) (FIG. 15E) and cyclo-WW+UV (FIG. 15F). Scale bar: 2 μm.

FIGS. 16A-B present a UV-Vis absorption spectra of cyclo-WW self-assemblies in the absence or presence of Zn(II) in MeOH (FIG. 16A), showing a new peak at 515 nm appeared when complexing cyclo-WW and Zn(II); and photographic presentation of the color change of dried cyclo-WW before and after coordination with Zn(II) (FIG. 16B), showing cyclo-WW assemblies after MeOH evaporation (left), and cyclo-WW+Zn(II) after MeOH evaporation (right). After complexation with Zn(II), the color changed from the original white/light yellow into pink.

FIG. 17 presents Job Plot analysis of cyclo-WW with Zn(II) at different ratios, with the total molar concentration fixed at 15.0 mM. The lines were added for guideline, showing the intersection point at cyclo-WW proportion of 0.7.

FIGS. 18A-B show a presentation of the chemical shifts of cyclo-WW hydrogen atoms upon coordination with Zn(II), compared to the peptide alone; the hydrogen atoms in different chemical environments are marked with italicized letters (FIG. 18A), and an FTIR spectra of cyclo-WW self-assemblies in the absence or presence of metal ion/UV irradiation (FIG. 18B); the peaks of the active bonds are numerically marked; the IR spectra were vertically moved for clarity.

FIG. 19 is a schematic presentation showing the possible molecular mechanism of cyclo-WW dimer coordination with Zn(II): the backbone diketopiperazine rings contribute to the complexation through nitrogen atoms, while the side-chain indole rings form aromatic interactions.

FIGS. 20A-C present plots showing the time-resolved fluorescent emission extracted from fluorescence spectra of cyclo-WW+Cu(II) excited at 370 nm (red) or at 395 (black) (FIG. 20A); of cyclo-WW+UV excited at 370 nm (FIG. 20B); and of cyclo-WW+Zn(II) MeOH solution at 520 nm (FIG. 20C), with the inset showing the fluorescent color evolution over time. The extracted maximal emissions are plotted against time (d: day; w: week.).

FIG. 21 presents MS spectra of cyclo-WW+Cu(II) and cyclo-WW+Cu(II)+UV, showing the MW of oxidized cyclo-WW (marked in red) and reduced Cu(I), confirming the redox reactions in the solutions.

FIG. 22A-B present the fluorescent emission spectra of cyclo-WW+Ag(I) and cyclo-WW+[AuCl₄](-I) (FIG. 22A), with the insets showing the fluorescent color of the sample solutions under UV light (365 nm), demonstrating that cyclo-WW+Ag(I) and cyclo-WW+[AuCl₄](-I) had the same emissions as cyclo-WW+Cu(II); and photos demonstrating the reduced Ag and Au metals adsorbed on the vial walls (FIG. 22B).

FIG. 23A-B present the fluorescent emission spectra of cyclo-FW self-assemblies in the absence or presence of Zn(II), Cu(II) and UV irradiation (FIG. 23A); and photos showing the color of the solutions under UV light (365 nm) (FIG. 23B).

FIG. 24 presents the fluorescent spectra of UV-irradiated (365 nm) cyclo-WW in the absence or presence of metal ions. The similar emission spectra indicated that the oxidized cyclo-WW could not complex with metal ions.

FIG. 25 presents a molecular excitation spectra of cyclo-WW in MeOH (black), and of cyclo-WW+Zn(II) in MeOH (blue) and in DMSO (red), showing that the molecular excitation red-shifted from 305 nm in MeOH to 310 nm in DMSO (emission set at 350 nm), and thus demonstrating that cyclo-WW+Zn(II) self-assembled more extensively in DMSO than in MeOH.

FIGS. 26A-C present a TEM image of self-assembled cyclo-WW+Zn(II) nanospheres (FIG. 26A; Scale bar: 300 nm); a bar graph showing a statistical diameter distribution of cyclo-WW+Zn(II) nanospheres in DMSO, as analyzed from TEM images, showing a diameter of 63.6±12.2 nm (FIG. 26B), and powder XRD spectrum of the nanospheres formed by cyclo-WW+Zn(II) in DMSO (FIG. 26C), in which the distinct sharp peaks and high intensities indicate well-ordered nanocrystal structures within the self-assemblies.

FIGS. 27A-C present an emission vs. excitation profile of the nanospheres formed of cyclo-WW+Zn(II) in DMSO (FIG. 27A), the extracted emission spectra of the nanospheres (FIG. 27B), and visible to NIR photos of cyclo-WW+Zn(II) DMSO solution under various wavelengths.

FIGS. 28A-B present photobleaching (FIG. 28A) and photostability (FIG. 28B) characterization of cyclo-WW+Zn(II) nanoparticles formed in DMSO (abbreviated as cPNPs) and of the fluorescent dyes ICG and Cy5.5, as controls.

FIGS. 29A-B present a schematic presentation of a LED setup using dried cyclo-WW+Zn(II) assembled in MeOH as phosphors (FIG. 29A), with the upper inset showing the working depiction of a prototype, emitting bright green light (Ex: 450 nm), and spectroscopic characterization of the LED photoluminescence using three excitation wavelengths, as indicated, showing the same emission at 550 nm (FIG. 29B).

FIGS. 30A-B present a bar graph showing the data obtained for the cytotoxicity of the cyclo-WW+Zn(II) nanospheres in DMSO (62.5 nM and 125 nM) towards B16-BL6, HaCaT and MCF7 cells (FIG. 30A; Viability relative to untreated controls ±sd, designated as error bars, is shown based on three repeats and averaged), and In vivo whole body NIR fluorescent imaging following subcutaneous injection of the nanospheres (50 μL, 2.7 mM) into nude mice, showing notable emissions under various excitations (FIG. 30B; The dotted circle indicates the location of injection).

FIGS. 31A-G present the 2D chemical structures of cyclo-HH, cyclo-FF and cyclo-YY (FIG. 31A); Microscopic images showing the self-assemblies of cyclo-HH in MeOH (FIG. 31B, scale bar: 1 μm), cyclo-FF in DMSO (FIG. 31C, scale bar: 50 nm), and cyclo-YY in DMSO (FIG. 31D, scale bar: 500 nm); and Fluorescent emission of cyclo-HH in MeOH (FIG. 31E), cyclo-FF in DMSO (FIG. 31F), and cyclo-YY in DMSO (FIG. 31G).

FIGS. 32A-C present AFM images showing the morphologies of self-assembled formed of cyclo-HH in DMSO (FIG. 32A, Scale bar: 600 nm) cyclo-FF in DMSO (FIG. 32B, Scale bar: 400 nm), and cyclo-YY in DMSO (FIG. 32C, Scale bar: 500 nm), at 0.5 mM.

FIG. 33 presents a general structure of exemplified aromatic cyclo-dipeptides featuring diketopiperazine skeleton according to some embodiments of the present invention. The different combinations of the R1 and R2 side chains give rise to 10 aromatic cyclo-dipeptides, as detailed in the table (right).

FIGS. 34A-L present AFM images of aromatic cyclo-dipeptides self-assemblies in MeOH. (FIG. 34A) cyclo-HH nanofibers. (FIG. 34B) cyclo-YY ribbons. (FIG. 34C) cyclo-WW nanospheres. (FIG. 34D) cyclo-FF nanofibers. (FIG. 34E) cyclo-WY nanofibers. (FIG. 34F) cyclo-HF nanofibers. (FIG. 34G) cyclo-FY fibers. (FIG. 34H) cyclo-FW ribbons. (FIG. 34I) cyclo-HY nanofibers. (FIG. 34J) cyclo-WH nanofibers. (FIG. 34K) cyclo-HY dot-like nanoparticles. (FIG. 34L) cyclo-WH dot-like nanoparticles. (FIG. 34I) and (FIG. 34K), (FIG. 34J) and (FIG. 34L) were obtained from the same samples, respectively. The height profile in each panel corresponds to the black line in the AFM image above.

FIGS. 35A-J present the Contour profiles of emission vs. excitation of cyclo-dipeptide MeOH solutions. (FIG. 35A) cyclo-HH. (FIG. 35B) cyclo-YY. (FIG. 35C) cyclo-WW. (FIG. 35D) cyclo-FF. (FIG. 35E) cyclo-HY. (FIG. 35F) cyclo-WH. (FIG. 35G) cyclo-WY. (FIG. 35H) cyclo-FH. (FIG. 35I) cyclo-FY. (FIG. 35J) cyclo-FW. The maximal emissions were extracted and are presented in Table 1.

FIGS. 36A-C present the fluorescence contour profiles of cyclo-HH in the absence (FIG. 36A) or presence (FIG. 36B) of Zn(II), with the insets showing photographic pictures of the corresponding peptide solutions under UV light irradiation, and the extracted maximal emission spectra of cyclo-HH in the absence (black) or presence (red) of Zn(II) ions (FIG. 36C).

FIG. 37A-B present an AFM image of cyclo-HH+Zn(II), in which the height profile in the lower panel corresponds to the black line in the AFM image, showing dot-like nanoparticles of only several nanometers (FIG. 37A), and DLS profile of cyclo-HH in the absence (black) or presence (red) of Zn(II) ions (FIG. 37B).

FIGS. 38A-D present FTIR spectra of cyclo-HH in the absence (black curve) or presence (red curve) of Zn(II). The —NH and —C—C stretching vibration regions are boxed by green and blue dashed rectangles, respectively (FIG. 38A), the 2D chemical structure of cyclo-HH, with the hydrogen atoms in different chemical environments marked with italicized alphabet letters (FIG. 38B); ¹H NMR spectrum of cyclo-HH in the absence (blue) or presence (red) of Zn(II) (FIG. 38C), and the chemical shifts of hydrogen atoms upon doping with Zn(II), compared to the peptide alone (FIG. 38D).

FIGS. 39A-B present graphic images of “Class 1” and “Class 2” β-bridge like conformations of cyclo-HH dimers (FIG. 39A), and graphic images showing the self-assembly procedure of cyclo-HH+Zn(II) into “Class 2” β-bridge like conformations, with the frequency percentage shown above the arrows (FIG. 39B). The cyclo-HH molecules and Zn(II) ions are shown in licorice and gray van der Waals representations, respectively. Hydrogen bonds and Zn(II) coordination are indicated with thin black lines.

FIGS. 40A-D present a photographic picture depicting the prototype of cyclo-HH+Zn(II) LED, emitting bright green light upon excitation at 420 nm (FIG. 40A); CIE coordinates of the operating LED and the corresponding color temperature (FIG. 40B); an emission spectrum of the LED operated under a voltage of 3.0 V (FIG. 40C); and an emission spectrum of a LED having cyclo-HH assemblies applied, operated under a voltage of 3.0 V, and showing no significant emission at wavelengths above 500 nm (FIG. 40D).

FIGS. 41A-C present an AFM image of self-assembled CHH—Zn, showing the presence of about 30 nm nanoparticles (FIG. 41A, Scale bar=400 nm), CHH—Zn TEM image (FIG. 41B), and dynamic light-scattering (DLS) measurement profiles of CHH—Zn (FIG. 41C).

FIGS. 42A-D present normalized UV-vis spectra of CHH—Zn, CHH and Zn(NO₃)₂, with the inset showing CHH—Zn under daylight (left) and UV lamp (365 nm; right)) (FIG. 42A); Excitation-emission matrix contour profiles of CHH—Zn (FIG. 42B); Trajectory of tunable fluorescence emission colors recorded upon changing the excitation wavelength of CHH—Zn from 330 to 450 nm in the CIE coordinate diagram (FIG. 42C); and schematic comparison of the quantum yield of different fluorescent biometabolites, with CHH—Zn marked with a green star (FIG. 42D).

FIGS. 43A-B present ¹H chemical shifts of CHH—Zn, compared to the peptide alone, obtained in NMR analysis (FIG. 43A) and a Job plot analysis of CHH with Zn(NO₃)₂ (FIG. 43B).

FIGS. 44A-B present a Single-crystal structure of CHH—Zn(II) in Pbcn space group, Color scheme: grey, C; red, O; blue, N; green, Zn, and purple, Iodine (left) and of CHH—NaNO₃ in P21/c space group (right) (FIG. 44A), and PXRD pattern of a CHH—Zn (red), the measured (green) and the simulated (blue) pattern of the single crystal structure and of CHH—Zn(NO₃)₂ (cyan) (FIG. 44B).

FIGS. 45A-D present data obtained in mechanistic analysis of CHH self-assembly with Zn(NO₃)₂. FIG. 45A presents a molecular graphics image of the CHH—Zn(II) elementary structure observed in MD simulations. The CHH are shown in licorice representation. Zinc ions are shown in yellow VDW representation. Hydrogen bonds and zinc coordination are indicated with black dotted lines. FIG. 45B present plots showing radius of gyration (Å) of Zn(II) ions within the clusters observed in the simulations of CHH—Zn(II) (blue) or CHH—Zn(II)+NO₃— (orange). FIG. 45C is a bar graph showing the percentage of CHH, NO₃—, and Zn(II) within clusters observed in the simulations of CHH—Zn(II)—NO₃. FIG. 45D is a schematic illustration of the plausible self-assembly process of CHH—Zn assemblies.

FIGS. 46A-B present photographs of CHH—Zn used as a phosphor for working green LED with a luminous efficiency of 54.69 lm/W, with the insert showing emission spectrum of working LED (FIG. 46A), and of OLED structure, energy diagram, and operation (FIG. 46B).

FIGS. 47A-C present confocal microscopy imaging of live cells treated with CHH—Zn and DRAQ5 (FIG. 47A; Scale bar is 25 μm), Z-Stack 3D image of HeLa cells (left) and the XY section image visualized through 3D reconstruction (right) (FIG. 47B), and a bar graph showing HeLa cells viability following incubation with different concentrations of the CHH—Zn+EPI carrier (0-4 μg/mL).

FIGS. 48A-D present confocal fluorescence images of HeLa cells incubated with CHHZn+Epirubicin and Epirubicin alone (FIG. 48A), plots showing profiles of Epirubicin release from CHH—Zn in 3.5 kDa dialysis chambers with different pH values (pH 6.0, or 7.4) (FIG. 48B), FLIM analysis of HeLa cells after incubation with CHH—Zn+Epirubicin, (I) Bright field (II) FLIM images and (III) phasor-separated and pseudocolored FLIM images of HeLa cells (FIG. 48C) and fluorescence lifetime histogram of Epirubicin at different time points (FIG. 48D, Scale bar is 25 μm).

FIGS. 49A-B are schematic illustrations of a light emitting system, according to various exemplary embodiments of the present invention.

FIG. 50 is a schematic illustration of a utility system according to various exemplary embodiments of the present invention.

FIGS. 51A-C are schematic illustrations of a system for analyzing a target material by two photon absorption, according to some embodiments of the present invention.

FIG. 52 is a schematic illustration of a communication system according to some exemplary embodiments of the present invention.

FIG. 53 is a schematic illustration of a quantum computer system according to some embodiments of the present invention.

FIG. 54 schematic illustration of a memory system, according to some embodiments of the present invention.

FIG. 55 is a schematic illustration of a memory system in embodiments of the invention in which the system operates according to the operation principle of a transistor.

FIGS. 56A-F are schematic illustrations of exemplary characteristic bandgap diagrams of a self-assembled structure formed of a plurality of cyclic peptides, according to some embodiments of the present invention.

### DESCRIPTION OF SPECIFIC EMBODIMENTS OF THE INVENTION

The present invention, in some embodiments thereof, relates to peptide-based materials and, more particularly, but not exclusively, to quantum confined peptide assemblies which feature tunable photoluminescence and to uses thereof as, for example, photoactive materials and carriers for drug delivery.

Before explaining at least one embodiment of the invention in detail, it is to be understood that the invention is not necessarily limited in its application to the details of construction and the arrangement of the components and/or methods set forth in the following description and/or illustrated in the drawings and/or the Examples and/or to the details set forth in the following description or exemplified by the Examples. The invention is capable of other embodiments or of being practiced or carried out in various ways.

Quantum confined (QC) materials have been extensively studied for photoluminescent applications. Due to intrinsic limitations of low biocompatibility and challenging modulation, the utilization of conventional inorganic quantum confined photoluminescent materials in bio-imaging and bio-machine interface faces critical restrictions.

In a search for biocompatible, organic, tunable and flexible QC alternatives with improved and stable photoluminescence, the present inventors have extensively studied the mechanism and properties of self-assemblies formed of aromatic cyclo-dipeptides. The present inventors have uncovered that aromatic cyclo-dipeptides dimerize into quantum dots, which serve as building blocks to further self-assemble into quantum confined supramolecular structures with diverse morphologies and photoluminescence properties. The present inventors have uncovered that the emission exhibited by these structures can be tuned from the visible to the near-infrared region (420 nm to 820 nm) by modulating the self-assembly process (e.g., by performing the process in the presence of metal salts), and have demonstrated that no cytotoxic effect is observed for these nanostructures, and their utilization for in vivo imaging and as phosphors for light-emitting diodes. The present inventors have uncovered that the morphologies and optical properties of the aromatic cyclo-dipeptide self-assemblies can be tuned, making them potential candidates for supramolecular quantum confined materials providing biocompatible alternatives for broad biomedical and opto-electric applications.

As shown in the Examples section that follows, it has been demonstrated that aromatic (including heteroaromatic) cyclo-dipeptides self-assemble to dimeric QDs (quantum dots), which act as building blocks to further organize into supramolecular structures. Due to the extensive and directional QC regions within, the assemblies show intrinsic photoluminescence properties. Through amino acid substitution, metal ion coordination, molecular oxidation or UV irradiation and/or solvent replacement, the supramolecular morphologies could be finely controlled, ranging from dimeric QDs to larger organizations. Correspondingly, the photoluminescence could be tuned, covering most of the visible into the NIR spectral region. The aromatic cyclo-dipeptides oligomerize into quantum dots, which act as building blocks to self-assemble into quantum-confined supramolecular structures with intrinsic fluorescence. In particular, the supramolecular morphologies could be finely tuned from dimeric quantum dots to larger organizations (needle-like crystals, nanospheres, nanofibers, nanorods et al.), by controlling the self-assembly process including substitution of aromatic amino acid residues, coordination with metal (e.g., zinc) ions, substitution with D-type enantiomers, oxidation by copper ions or UV irradiation. Correspondingly, the photoluminescence could be modulated in a wide-spectrum visible light region.

The self-assembly could be finely halted at an initial oligomerization step by doping (coordinating) with metal (e.g., zinc) ions, through attracting and pulling the metal ions from the solvent into peptide domain. The doping significantly enhances the emissions.

The biocompatibility and wide-spectrum emission features make these supramolecular structures highly suitable for in vivo bio-imaging applications with no detected cytotoxicity and for the fabrication of light emitting configurations such as LEDs, where the assemblies are used as phosphors.

By thorough analysis of aromatic cyclo-dipeptide self-assembly, the enhancement of fluorescence via coordination with Zn(II) was demonstrated. The metal-ligand electron transfer resulted in redshifted and enhanced emissions with high quantum yields (QYs).

Diverse modulation strategies, such as design of larger cyclo-oligopeptides, substitution with D-type enantiomers, complexation with other metal ions, flexible assembly approaches, self-assembly in different solvents etc., were shown to tune the cyclo-peptides self-assemblies, providing diverse QC supramolecular structures with a rainbow of photoluminescence in visible and even infrared region.

The present inventors have further constructed a self-assembled structure of a fluorescent short peptide core encapsulated by the peptide scaffold building module, which exhibits a bright fluorescence with quantum yields of up to 70% for green fluorescence, and have demonstrated the utilization of these bright fluorescence peptide self-assemblies for eco-friendly optoelectronics and bioimaging. The “self-encapsulation” strategy was utilized for fabricating an advanced nanocarrier for traceable intracellular drug delivery.

Some embodiments of the present invention relate to self-assembled, quantum-confined, photoluminescent supramolecular structures (e.g., nanostructures) which comprise self-assembled aromatic cyclo-peptides (e.g., aromatic cyclic dipeptides) which can comprise, for example, one or more of histidine (His, H), tyrosine (Tyr, Y), tryptophan (Trp, W) and phenylalanine (Phe, F), or of non-coded structurally analogous amino acids, as detailed hereinbelow, optionally in combination with metal ions or metal salts.

Some embodiments of the present invention relate to uses of these peptide structures in materials science, quantum research, nanotechnology, fundamental biology, biomedicine, nanotechnology and bio-organic optical and electronic fields.

Self-assembled structure: A self-assembled structure as described herein is also referred to herein as self-assembled peptide structure. The self-assembled structure is composed of a plurality of molecules, that is, peptide molecules as described herein and optionally metal ions (e.g., metal salts), which assemble together to form a three-dimensional (e.g., at least partially ordered) structure. The peptide molecules are linked to one another by non-covalent bonds, preferably via π-π aromatic interactions. When metal ions form a part of the structure, the metal ions are linked to the peptide molecules or a portion thereof by coordinative interactions.

The self-assembled structure is typically formed spontaneously (self-assemble) when the plurality of molecules (e.g., cyclic peptides and optionally metal salt) are contacted together and subjected to conditions that allow self-assemble to occur. Such conditions typically include contacting the molecules in the presence of a suitable solvent, at a concentration that allows self-assemble to occur, as described in further detail hereinafter and exemplified in the Examples section that follows.

In some of any of the embodiments described herein, the self-assembled structure is a self-assembled nanostructure, that is, a structure that has an average size of less than 1 micrometer, or less than 500 nm, or less than 100 nm, of at least one dimension or cross-section thereof. According to an aspect of some embodiments of the present invention there is provided a self-assembled structure formed of a plurality of cyclic peptides.

The plurality of cyclic peptides can comprise two cyclic peptides, which form a dimer as the self-assembled structure. In some of these embodiments, the self-assembled dimers are in a form of quantum dots (QDs).

The plurality of cyclic peptides can comprise three, four, five, six, seven, eight, nine, ten, or more cyclic peptides, and can comprise dozens, hundreds and even more cyclic peptides that self-assemble to form the structure. The cyclic peptides can be the same or different and are preferably the same.

The self-assembled structure of the present embodiments can adopt various sizes and shapes, which can be manipulated by the choice of the cyclic peptides, and/or by the conditions to which the self-assembly is subjected (e.g., solvent, cyclic peptide's concentration). The size and shape of the structure can affect the photoluminescence performance of the structure and can be manipulated so as to provide a desirable performance.

In some of any of the embodiments described herein, all the cyclic peptides in the plurality of cyclic peptides are the same.

According to some embodiments, each cyclic peptide in the plurality of cyclic peptides is independently a cyclic short peptide which comprises up to 10 amino acid residues, preferably from 2 to 6 amino acid residues.

In some of any of the respective embodiments, at least a portion, or each, of the plurality of cyclic peptides comprises cyclic peptides of 2 to 10 amino acid residues, optionally from 2 to 9 amino acid residues, optionally from 2 to 8 amino acid residues, optionally from 2 to 7 amino acid residues, optionally from 2 to 6 amino acid residues, optionally from 2 to 5 amino acid residues, and optionally from 2 to 4 amino acid residues. In exemplary embodiments, at least a portion, or each, of said plurality of cyclic peptides comprises 2 or 3 amino acid residues. In some of any of the aforementioned embodiments, each amino acid reside is an α-amino acid residue.

Herein throughout, by “at least a portion” it is meant at 10%, or at least 20%, or at least 30%, preferably at least 50 5, or at least 60%, or at least 70%, or at least 80%, or at least 90%, or all of the cyclic peptides in the plurality of cyclic peptides.

According to some of any of the embodiments described herein, at least one, preferably at least two, and optionally all, of the amino acid residues forming the cyclic peptide is/are aromatic amino acid residue(s), as described herein. When two or more aromatic amino acid residues are present, the aromatic amino acid residues can be the same or different.

According to some of any of the embodiments described herein, in at least a portion of the plurality of cyclic peptides, each cyclic peptide is a cyclic dipeptide, comprised of two amino acid residues. In some of these embodiments, each of the two amino acid residues is independently an aromatic amino acid residue. The two aromatic amino acid residues can be the same or different. According to some of any of the embodiments described herein, the presence of aromatic amino acid residues in the cyclic peptide allows the plurality of cyclic peptides to self-assemble so as to form a supramolecular structure.

The term “peptide” as used herein encompasses native peptides (either degradation products, synthetically synthesized peptides or recombinant peptides) and peptidomimetics (typically, synthetically synthesized peptides), as well as peptoids and semipeptoids which are peptide analogs, which may have, for example, modifications rendering the peptides more stable while in a body or more capable of penetrating into cells. Such modifications include, but are not limited to, N-terminus modification, C-terminus modification, peptide bond modification, including, but not limited to, CH₂—NH, CH₂—S, CH₂—S═O, O═C—NH, CH₂—O, CH₂—CH₂, S═C—NH, CH═CH or CF═CH, backbone modifications, and residue modification. Methods for preparing peptidomimetic compounds are well known in the art and are specified, for example, in Quantitative Drug Design, C. A. Ramsden Gd., Chapter 17.2, F. Choplin Pergamon Press (1992). Peptide bonds (—CO—NH—) within the peptide may be substituted, for example, by N-methylated bonds (—N(CH₃)—CO—), ester bonds (—C(R)H—C—O—O—C(R)—N—), ketomethylene bonds (—CO—CH₂—), α-aza bonds (—NH—N(R)—CO—), wherein R is any alkyl, e.g., methyl, carba bonds (—CH₂—NH—), hydroxyethylene bonds (—CH(OH)—CH₂—), thioamide bonds (—CS—NH—), olefinic double bonds (—CH═CH—), retro amide bonds (—NH—CO—), peptide derivatives (—N(R)—CH₂—CO—), wherein R is the “normal” side chain, naturally presented on the carbon atom. These modifications can occur at any of the bonds along the peptide chain and even at several (2-3) at the same time.

In any of the respective embodiments herein pertaining to a cyclic peptide, each of the amino acid residues of the cyclic peptide may independently be a coded amino acid residue or a non-coded amino acid residue. Herein, a “coded” amino acid refers to any of the 20 “standard” amino acids encoded by the universal genetic code.

As used herein throughout, the term “amino acid” or “amino acids” is understood to include the 20 naturally occurring amino acids, which are also referred to herein as “coded” amino acids; those amino acids often modified post-translationally in vivo, including, for example, hydroxyproline, phosphoserine and phosphothreonine; and other unusual amino acids, including synthetically prepared amino acids, including, but not limited to, 2-aminoadipic acid, hydroxylysine, isodesmosine, nor-valine, nor-leucine and ornithine. The term “amino acid” includes both D- and L-amino acids.

In any of the respective embodiments herein in which the chirality of one or more amino acid residues of a peptide is not explicitly defined, each of the amino acid residues is an L-amino acid residue.

Natural aromatic amino acids, Trp, Tyr, His and Phe, may be substituted for synthetic unnatural acids such as phenylglycine, TIC, naphthylalanine (Nal), ring-methylated derivatives of Phe, halogenated derivatives of Phe or O-methyl-Tyr, imidazole-substituted derivatives of His, and β amino-acids. Such modified amino acids are also referred to herein as structural analogs of the aromatic amino acids.

In some of any of the embodiments described herein, at least a portion of the cyclic peptides described herein comprises polyaromatic cyclic peptides, comprising two or more aromatic amino acid residues. In some embodiments, at least a portion of the cyclic peptides described herein comprises or consists essentially of aromatic amino acid residues. In some embodiments, each cyclic peptide in the plurality of cyclic peptides consists essentially of aromatic amino acid residues.

The cyclic peptides described herein can include any combination of: cyclic dipeptides composed of one or two aromatic amino acid residues; cyclic tripeptides including one, two or three aromatic amino acid residues; cyclic tetrapeptides including two, three or four aromatic amino acid residues; cyclic pentapeptides including two, three, four or five aromatic amino acid residues; and cyclic hexapeptides including two, three, four, five or six aromatic amino acid residues.

The phrase “aromatic amino acid residue”, as used herein, refers to an amino acid residue that comprises an aromatic moiety in its side-chain.

As used herein, the phrase “aromatic moiety” describes a monocyclic or polycyclic moiety having a completely conjugated pi-electron system. The aromatic moiety can be an all-carbon moiety (aryl) or can include one or more heteroatoms such as, for example, nitrogen, sulfur or oxygen (heteroaryl). The aromatic moiety can be substituted or unsubstituted, whereby when substituted, the substituent can be, for example, one or more of alkyl, trihaloalkyl, alkenyl, alkynyl, cycloalkyl, aryl, heteroaryl, heteroalicyclic, halo, nitro, azo, hydroxy, alkoxy, thiohydroxy, thioalkoxy, cyano and amine. The aromatic moiety can include one or more aryl and/or heteroaryl groups, as defined hereinbelow, which can be fused or non-fused to one another.

Exemplary aromatic moieties include, but are not limited to, phenyl, biphenyl, naphthalenyl, phenanthrenyl, anthracenyl, [1,10]phenanthrolinyl, indoles, imidazoles, thiophenes, thiazoles and [2,2′]bipyridinyl, each being optionally substituted. Thus, representative examples of aromatic moieties that can serve as the side chain within the aromatic amino acid residues described herein include, without limitation, substituted or unsubstituted naphthalenyl, substituted or unsubstituted phenanthrenyl, substituted or unsubstituted anthracenyl, substituted or unsubstituted [1,10]phenanthrolinyl, substituted or unsubstituted [2,2′]bipyridinyl, substituted or unsubstituted biphenyl, and substituted or unsubstituted phenyl. The aromatic moiety can alternatively be substituted or unsubstituted heteroaryl such as, for example, indole, thiophene, imidazole, oxazole, thiazole, pyrazole, pyridine, pyrimidine, quinoline, isoquinoline, quinazoline, quinoxaline, and purine.

When substituted, the aromatic moiety includes one or more substituents such as, but not limited to, alkyl, trihaloalkyl, alkenyl, alkynyl, cycloalkyl, aryl, heteroaryl, heteroalicyclic, halo, nitro, azo, hydroxy, alkoxy, thiohydroxy, thioalkoxy, cyano, and amine. Exemplary substituted phenyls may be, for example, pentafluoro phenyl, iodophenyl, biphenyl and nitrophenyl.

Herein, a “cyclic peptide” is also referred to as “cyclo-peptide”, whereby specific peptides are preceded by the prefix “cyclo-” or “cyclic”.

A cyclic peptide according to any of the respective embodiments described herein may optionally be a cyclic peptide obtainable by linking a peptide C-terminus to a peptide N-terminus by an amide bond, by linking two side-chains (e.g., cysteine side chains) by a disulfide (—S—S—) bond, by a lactam bridge, by a hydrocarbon-staple (optionally a chiral hydrocarbon staple), by a triazole bridge, by bio-Cys alkylation, or by an acetone Hcy linker, and/or by any form of peptide cyclization described in the art, e.g., in Hu et al. [Angew. Chem. Int. Ed. 55:8013-8017 (2016)].

In exemplary embodiments, a cyclic peptide as described herein is a peptide in which a peptide's C-terminus is linked to its N-terminus by an amide bond.

In some of any of the respective embodiments, at least one of the amino acid residues (in at least a portion, or each, of the plurality of cyclic peptides) comprises an aromatic moiety. In some embodiments, at least two adjacent amino acid residues (in at least a portion, or each, of the plurality of cyclic peptides) each comprise an aromatic moiety. Examples of amino acid residues comprising an aromatic moiety include, without limitation, residues of phenylalanine (Phe), tyrosine (Tyr), tryptophan (Trp), histidine (His), β,β-diphenylalanine (Dip), naphthylalanine (Nal), and dihydroxyphenylalanine (DOPA).

In any of the respective embodiments herein, a cyclic peptide is a cyclic dipeptide, e.g., a substituted diketopiperazine.

Each of these cyclic dipeptides can include one or two aromatic amino acid residues. Preferably, each of these dipeptides includes two aromatic amino acid residues. The aromatic residues composing the cyclic dipeptide can be the same, such that the cyclic dipeptide is a cyclic homodipeptide, or different.

The phrase “aromatic cyclic dipeptide” as used herein describes a cyclic peptide composed of two amino acid residues, at least one, and preferably both, being an aromatic amino acid as defined herein.

According to some of any of the embodiments described herein, the aromatic cyclic dipeptide comprises in its side chain an aromatic group which is unsubstituted or which is substituted by one or more substituents as described herein.

According to some of any of the embodiments described herein, at least a portion of, or each cyclic peptide, in the plurality of cyclic peptides is an aromatic cyclic dipeptide. According to some of any of the embodiments described herein, at least a portion of, or each cyclic peptide, in the plurality of aromatic cyclic dipeptides comprises a plurality of aromatic dipeptides of two aromatic amino acid residues as described herein.

According to some of any of the embodiments described herein, at least a portion of, or each cyclic peptide in, the plurality of aromatic cyclic dipeptides comprises aromatic homodipeptides, having two aromatic amino acid residues which are identical with respect to their side-chains residue, or in which the two aromatic amino acid residues are identical (the same).

Exemplary aromatic cyclic homodipeptides include, but are not limited to, phenylalanine-phenylalanine cyclic dipeptide, naphthylalanine-naphthylalanine cyclic dipeptide, (pentafluro-phenylalanine)-(pentafluro-phenylalanine) cyclic dipeptide, (iodo-phenylalanine)-(iodo-phenylalanine) cyclic dipeptide, (4-phenyl phenylalanine)-(4-phenyl phenylalanine) cyclic dipeptide, (p-nitro-phenylalanine)-(p-nitro-phenylalanine) dipeptide, tryptophan-tryptophan cyclic dipeptide, tyrosine-tyrosine cyclic dipeptide, and histidine-histidine cyclic dipeptide.

According to some of any of the embodiments described herein, each of the aromatic homodipeptides is a (substituted or unsubstituted) phenylalanine-phenylalanine dipeptide.

According to some of any of the embodiments described herein, each of the aromatic homodipeptides is an unsubstituted phenylalanine-phenylalanine dipeptide (cyclo-Phe-Phe; cyclo-FF). In some of these embodiments, the self-assembled structure is in a form of a nanofiber (e.g., a plurality of nanofibers).

According to some of any of the embodiments described herein, each of the aromatic homodipeptides is an unsubstituted tryptophan-tryptophan dipeptide (cyclo-Trp-Trp; cyclo-WW). In some of these embodiments, the self-assembled structure is in a form of a nanospheres (e.g., a plurality of nanospheres).

According to some of any of the embodiments described herein, each of the aromatic homodipeptides is an unsubstituted tyrosine-tyrosine dipeptide (cyclo-Tyr-Tyr; cyclo-YY). In some of these embodiments, the self-assembled structure is in a form of platelets (e.g., a plurality of nanoplatelets).

According to some of any of the embodiments described herein, each of the aromatic homodipeptides is an unsubstituted histidine-histidine dipeptide (cyclo-His-His; cyclo-HH). In some of these embodiments, the self-assembled structure is in a form of a nanofiber (e.g., a plurality of nanofibers).

According to some of any of the embodiments described herein, at least a portion of, or each cyclic peptide in, the plurality of aromatic cyclic dipeptides comprises aromatic heterodipeptides, having two aromatic amino acid residues which are different with respect to their side-chains residue, or in which the two aromatic amino acid residues are different with respect to their chirality.

Exemplary such aromatic cyclic dipeptides include, but are not limited to, phenylalanine-tryptophan cyclic dipeptide, naphthylalanine-tryptophan cyclic dipeptide, (pentafluro-phenylalanine)-tryptophan cyclic dipeptide, (iodo-phenylalanine)-tryptophan cyclic dipeptide, (4-phenyl phenylalanine)-tryptophan cyclic dipeptide, (p-nitro-phenylalanine)-tryptophan dipeptide, phenylalanine-tyrosine cyclic dipeptide, naphthylalanine-tyrosine cyclic dipeptide, (pentafluro-phenylalanine)-tyrosine cyclic dipeptide, (iodo-phenylalanine)-tyrosine cyclic dipeptide, (4-phenyl phenylalanine)-tyrosine cyclic dipeptide, (p-nitro-phenylalanine)-tyrosinedipeptide, phenylalanine-histidine cyclic dipeptide, naphthylalanine-histidine cyclic dipeptide, (pentafluro-phenylalanine)-histidine cyclic dipeptide, (iodo-phenylalanine)-histidine cyclic dipeptide, (4-phenyl phenylalanine)-histidine cyclic dipeptide, (p-nitro-phenylalanine)-histidine dipeptide, tryptophan-histidine cyclic dipeptide, tyrosine-tryptophan cyclic dipeptide, and histidine-tyrosine cyclic dipeptide.

The skilled person will appreciate that an indicated “first” amino acid of a cyclic peptide may be arbitrary, such that, e.g., cyclo-Phe-Trp may also be considered as cyclo-Trp-Phe.

According to some of any of the embodiments described herein, each of the aromatic cyclic dipeptides is a (substituted or unsubstituted) phenylalanine-tryptophan dipeptide.

According to some of any of the embodiments described herein, each of the aromatic cyclic dipeptides is an unsubstituted phenylalanine-tryptophan dipeptide (cyclo-Phe-Trp; cyclo-FW). In some of these embodiments, the self-assembled structure is in a form of a platelet (e.g., a plurality of nanoplatelets).

According to some of any of the embodiments described herein, each of the aromatic cyclic dipeptides is an unsubstituted phenylalanine-tyrosine dipeptide (cyclo-Phe-Tyr; cyclo-FY). In some of these embodiments, the self-assembled structure is in a form of a nanofiber (e.g., a plurality of nanofibers).

According to some of any of the embodiments described herein, each of the aromatic cyclic dipeptides is an unsubstituted phenylalanine-histidine dipeptide (cyclo-Phe-His; cyclo-FH). In some of these embodiments, the self-assembled structure is in a form of a nanofiber (e.g., a plurality of nanofibers).

According to some of any of the embodiments described herein, each of the aromatic cyclic dipeptides is an unsubstituted tyrosine-tryptophan dipeptide (cyclo-Tyr-Trp; cyclo-YW). In some of these embodiments, the self-assembled structure is in a form of a nanofiber (e.g., a plurality of nanofibers).

According to some of any of the embodiments described herein, each of the aromatic cyclic dipeptides is an unsubstituted histidine-tyrosine dipeptide (cyclo-His-Tyr; cyclo-HY). In some of these embodiments, the self-assembled structure is in a form of a nanofiber (e.g., a plurality of nanofibers).

According to some of any of the embodiments described herein, each of the aromatic cyclic dipeptides is an unsubstituted tryptophan-histidine dipeptide (cyclo-Trp-His; cyclo-WH). In some of these embodiments, the self-assembled structure is in a form of a nanofiber (e.g., a plurality of nanofibers).

According to some of any of the embodiments described herein, each of the aromatic cyclic dipeptides comprises a (substituted or unsubstituted) imidazole in its side chain.

In some of any of the embodiments described herein, for any of the above-mentioned aromatic cyclic dipeptides, each of the amino acid residues is L-amino acid residue.

In some of any of the embodiments described herein, for any of the above-mentioned aromatic cyclic dipeptides, each of the amino acid residues is D-amino acid residue.

In some of any of the embodiments described herein, for any of the above-mentioned aromatic cyclic dipeptides, one of the amino acid residues is D-amino acid residue and one of the amino acid residues is L-amino acid residue.

In some embodiments, all of the aromatic cyclic dipeptides in the plurality of cyclic peptides forming the self-assembled structures are the same, that is, all have the same amino acid residues, and the same type of peptide bond linking therebetween. In some of these embodiments, the amino acids residues have the same or different chirality.

According to an aspect of some embodiments of the present invention the self-assembled structure is formed of a plurality of cyclic peptides as described herein that comprises or consists of a plurality of cyclo-WW, or a plurality of cyclo-WF, or a plurality of cyclo-YY or a plurality of cyclo-FF.

According to an aspect of some embodiments of the present invention the self-assembled structure is formed of a plurality of cyclic peptides as described herein that comprises or consists of a plurality of cyclo-WW.

According to an aspect of some embodiments of the present invention the self-assembled structure is formed of a plurality of cyclic peptides as described herein comprises or consists of a plurality of cyclo-HH. According to some of these embodiments, both histidine residues are L-histidine residues. According to some of these embodiments, one of the histidine residues is L-histidine and one is D-histidine. Such a cyclic peptide is also referred to herein as CHH.

According to an aspect of some embodiments of the present invention the self-assembled structure is formed of a plurality of cyclic peptides as described herein that comprises or consists of a plurality of cyclic aromatic dipeptides as described herein, and at least a portion, or each, of the plurality of cyclic dipeptide comprise or consist of cyclic aromatic dipeptides (homodipeptides or heterodipeptides) that do not comprise a tryptophan residue.

According to an aspect of some embodiments of the present invention the self-assembled structure is formed of a plurality of cyclic peptides as described herein that comprises a plurality of cyclic aromatic dipeptides as described herein, and at least a portion, or each, of the plurality of cyclic dipeptides comprises or consists of cyclic aromatic heterodipeptides as described herein. According to an aspect of some embodiments of the present invention the self-assembled structure is formed of a plurality of cyclic peptides as described herein that comprises or consists of a plurality of cyclic aromatic dipeptides as described herein, and at least a portion, or each, of the plurality of cyclic dipeptide comprise or consist of cyclic aromatic heterodipeptides that do not comprise a tryptophan residue.

According to an aspect of some embodiments of the present invention the self-assembled structure is formed of a plurality of cyclic peptides as described herein (e.g., cyclic dipeptides, cyclic tripeptides and/or cyclic tetrapeptides), and at least a portion, or each, of the plurality of cyclic dipeptides comprises or consists of cyclic aromatic peptides as described herein, each comprising at least one aromatic amino acid that comprises an imidazole in its side-chain (e.g., histidine or a structural analog thereof). As exemplified in the Examples section that follows, it has been demonstrated that a presence of an imidazole group results in coordinative interactions with metal ions that provides an enhanced modulation of the photoluminescence properties of the self-assembled structure.

According to an aspect of some embodiments of the present invention the self-assembled structure is formed of a plurality of cyclic peptides as described herein that comprises a plurality of cyclic aromatic dipeptides as described herein, and at least a portion, or each, of the plurality of cyclic dipeptides comprises or consists of cyclic aromatic dipeptides (heterodipeptides or homodipeptides) as described herein, each comprising one aromatic amino acid that comprises an imidazole in its side-chain (e.g., histidine).

According to an aspect of some embodiments of the present invention the self-assembled structure is formed of a plurality of cyclic peptides as described herein, and at least a portion, or each, of the plurality of cyclic dipeptides comprises one L-amino acid residue (e.g., an aromatic L-amino acid residue) and one D-amino acid residue (e.g., aromatic D-amino acid residue).

According to an aspect of some embodiments of the present invention the self-assembled structure is formed of a plurality of cyclic peptides as described herein that comprises a plurality of cyclic aromatic dipeptides as described herein, and at least a portion, or each, of the plurality of cyclic aromatic dipeptides comprises or consists of cyclic aromatic dipeptides (heterodipeptides or homodipeptides) as described herein, each comprising one aromatic L-amino acid residue and one aromatic D-amino acid residue.

According to an aspect of some embodiments of the present invention the self-assembled structure is formed of a plurality of cyclic peptides as described herein (e.g., cyclic dipeptides, cyclic tripeptides and/or cyclic tetrapeptides), and at least a portion, or each, of the plurality of cyclic dipeptides comprises or consists of cyclic aromatic peptides as described herein, each comprising at least one aromatic amino acid that comprises an imidazole in its side-chain (e.g., histidine), and each comprising one L-amino acid residue (e.g., an aromatic L-amino acid residue) and one D-amino acid residue (e.g., aromatic D-amino acid residue). As exemplified in the Examples section that follows, it has been demonstrated that a presence of an imidazole group along with two amino acids of different chirality provides an enhanced modulation of the photoluminescence properties of the self-assembled structure.

According to an aspect of some embodiments of the present invention the self-assembled structure is formed of a plurality of cyclic peptides as described herein that comprises a plurality of cyclic aromatic dipeptides as described herein, and at least a portion, or each, of the plurality of cyclic dipeptides comprises or consists of cyclic aromatic dipeptides (heterodipeptides or homodipeptides) as described herein, each comprising one aromatic amino acid that comprises an imidazole in its side-chain (e.g., histidine), one of these aromatic amino acids is L-amino acid residue and one of these aromatic amino acids is D-amino acid residue.

According to an aspect of some embodiments of the present invention the self-assembled structure is formed of a plurality of cyclic peptides as described herein (e.g., cyclic dipeptides, cyclic tripeptides and/or cyclic tetrapeptides) which is devoid of tryptophan-containing cyclic peptides.

According to an aspect of some embodiments of the present invention the self-assembled structure is formed of a plurality of cyclic peptides as described herein (e.g., cyclic dipeptides, cyclic tripeptides and/or cyclic tetrapeptides), which is devoid of one or more of cyclo-WW, cyclo-YY, cyclo-FF, and cyclo-WF.

According to some of any of the embodiments described herein, in at least a portion, or in all, of the plurality of cyclic peptides, each amino acid residue has the same chirality (e.g., each amino acid residue is an L-amino acid residue).

According to some of any of the embodiments described herein, in at least a portion, or in all, of the plurality of cyclic peptides, in at least a portion of the plurality of cyclic peptides, at least one amino acid residue is an L-amino acid residue and at least one amino acid residue is a D-amino acid residue. According to some of these embodiments, at least a portion, or all, of the plurality of cyclic peptides, do not comprise a tryptophan residue.

According to an aspect of some embodiments of the present invention the self-assembled structure is formed of a plurality of cyclic aromatic homodipeptides as described herein.

According to some embodiments of the present invention, the self-assembled plurality of cyclic peptides as described herein in any of the respective embodiments exhibits photoluminescence. In some of these embodiments, the photoluminescence is exhibited upon exposure to light in the UV-vis region. In some of these embodiments, the photoluminescence is exhibited at room temperature.

Photoluminescence is a process in which a molecule absorbs a photon in the visible region, exciting one of its electrons to a higher electronic excited state, and then radiates a photon as the electron returns to a lower energy state. If the molecule undergoes internal energy redistribution after the initial photon absorption, the radiated photon is of longer wavelength (i.e., lower energy) than the absorbed photon. Photoluminescence encompasses fluorescence (when an excited electron is in a singlet state) and phosphorescence (when an excited electron is in a triplet state).

In some embodiments, the self-assembled plurality of cyclic peptides as described herein in any of the respective embodiments exhibits photoluminescence in response to excitation by light at a wavelength within the UV-vis spectral range.

In some embodiments, the self-assembled plurality of cyclic peptides as described herein in any of the respective embodiments exhibits photoluminescence by emitting light at a wavelength within the UV-vis spectral range. In some embodiments, the light emitted upon excitation at a wavelength lower than 400 nm is at a wavelength lower than 500 nm.

In some embodiments, the self-assembled plurality of cyclic peptides as described herein in any of the respective embodiments exhibits a quantum yield lower than 10, typically lower than 5, or lower than 4, e.g., of about 2 or 3.

By “quantum yield” it is meant the number of times a specific event occurs upon absorption of a photon by a system.

According to an aspect of some embodiments of the present invention there is provided a self-assembled structure which comprises a self-assembled plurality of cyclic peptides as described herein in any of the respective embodiments, wherein at least a portion of the cyclic peptides is in association with metal ions. In some embodiments, the metal ions are positively charged metal ions.

In some of these embodiments, the metal ions form a part of the self-assembled structures, and in some embodiments, the metal ions are co-assembled with the cyclic peptides. The association between the cyclic peptides and metal ions is typically via coordination bonds, presumably, but not explicitly, coordinative bonds between electron donating atoms or groups in the cyclic peptide and the metal ions.

In some embodiments, the electron donating atoms or groups can be of the amide bonds of the cyclic peptides. Preferably, the electron donating groups are of a side chain of the cyclic peptides, for example, of amine, hydroxy, thiols, alkoxy, thioalkoxy, and like nitrogen-, oxygen-or sulfur-containing substituents of the aromatic moiety in the side chain of aromatic amino acid residues in the cyclic peptide, or of a nitrogen, oxygen or sulfur heteroatom in an heteroaromatic moiety in the side chain. For example, the electron donating group can be a nitrogen of an indole-containing aromatic moiety (as in e.g., tryptophan) or of an imidazole-containing aromatic moiety (as in e.g., histidine).

In some of any of the embodiments described herein, the metal ions are multivalent metal ions, that is, are capable of forming two or more coordinative bonds, or, in other words, feature a positive charge higher than +1 or an oxidation state higher than (II).

The metal ions can be multivalent ions of any respective metal, including main group metals, transition metals, and elements of the lanthanide and actinide series. Non-limiting examples include zinc ions, copper ions, silver ions, gold ions, magnesium ions, manganese ions, cadmium ions, ferrous ions, and the like.

In exemplary embodiments, the metal ions are zinc ions.

In some embodiments, the metal ions are oxidizing ions, which are capable of being reduced to a lower oxidation state. Exemplary such ions include, but are not limited to, copper (II) ions, silver ions, gold ions, ferric ions, and the like.

According to some of any of the embodiments described herein, the metal ions form a part of a metal salt, which further comprises counter ions (anions). In some embodiments, the metal salt is dissolvable or dispersible in a solvent at which self-assembly occurs. The counter ion can be organic or inorganic. Exemplary counter ions include, but are not limited to, halides, nitrates, BF₃ etherate, etc.

In some of any of the embodiments described herein, a mol ratio between the metal ions and the cyclic peptides in the self-assembled structure ranges from about 5:1 to about 1:5 or from about 1:3 to about 3:1 or from about 2:1 to 1:2, or is about 1:1 (stoichiometric ratio).

In some of any of the embodiments described herein, the association with the metal ions affects the size and/or the shape of the self-assembled plurality of cyclic peptides, such that a size and/or a shape of structure that does not comprise metal ions is different from a size and/or a shape of structure that comprises the metal ions, as exemplified in the Examples section that follows. In some of any of the embodiments described herein, a self-assembled structure as described herein has an average size of less than 100 nm at least in one dimension or cross-section. The self-assembled structure can have an average size as defined herein of from few nanometers and up to 100 nm, including any intermediate values and subranges therebetween. The self-assembled structure is also referred to as a self-assembled nanostructure.

In some of any of the embodiments described herein the self-assembled structure is formed by contacting the plurality of cyclic peptides with metal ions in the presence of a solvent. The solvent is preferably a polar organic solvent such as an alcohol (e.g., methanol, ethanol, isopropanol, butanol, etc.), DMSO, DMF, and like solvents.

The concentration of the cyclic peptides is preferably such that allows self-assembly to occur, and can range, for example, from 0.1 mM to 100 mM, or from 0.1 mM to 50 mM, or from 0.1 mM to 20 mM, or from 0.1 mM to 10 mM, or from 0.1 mM to 5 mM, or from 5 mM to 20 mM, or from 0.5 mM to 100 mM, or from 0.5 mM to 50 mM, or from 0.5 mM to 20 mM, or from 0.5 mM to 10 mM, or from 0.5 mM to 5 mM, or from 5 mM to 20 mM, or from 1 mM to 100 mM, or from 1 mM to 50 mM, or from 1 mM to 20 mM, or from 1 mM to 10 mM, or from 1 mM to 5 mM, including any intermediate values and subranges therebetween. In exemplary embodiments, the concentration is higher than 0.5 Mm or higher than 1 mM.

The self-assembled structures as described herein can adopt various structural configurations, depending in the type of cyclic peptides and metal ions.

In exemplary embodiments, for example, when the metal ions are zinc ions, the self-assembled structures are dimeric structures, and are is a form of quantum dots. The self-assembled structures can form larger structures (e.g., nanofibers) upon, for example, solvent evaporation or upon applying other conditions.

In exemplary embodiments, at least a portion of the self-assembled structure is crystalline. In exemplary embodiments, the self-assembled structures features an ordered (e.g., crystalline) structure in its inner region (core) and an amorphous structure in its outer region (shell), thus forming a pseudo-core/shell structure. Without being bound by any particular theory, it is assumed that such a structural configuration provides for improved photoluminescence stability.

In some of any of the embodiments described herein, by being associated with the metal ions, at least one property of the photoluminescence of the self-assembled plurality of cyclic peptides is modulated.

Such a modulation in a photoluminescence property can be, for example, an emission wavelength (in response to the same excitation event), an excitation wavelength (for providing the same emission wavelength), the quantum yield, the photoluminescence time, and the photoluminescence stability.

In some embodiments, the association with the metal ions modulates an emission wavelength of the self-assembled plurality of cyclic peptides in response to the same excitation event (e.g., the same excitation wavelength).

In some embodiments, the emission wavelength is redshifted when the self-assembled cyclic peptides are in association with metal ions.

By “redshifted” it is meant that an increase in wavelength; a decrease in wave frequency and photon energy.

In some of any of the embodiments described herein, the self-assembled structure exhibits, upon excitation at a wavelength within the UV-vis spectral range (e.g., of from 300 nm to 400 nm), an emission wavelength of higher than 400 nm, higher than 450 nm, and even higher than 500 nm.

Alternatively, and depending on the type of metal ions associated with the plurality of cyclic peptides, other photoluminescence properties are modulated. Exemplary such modulations are demonstrated in the Examples section that follows, and include, for example, association with oxidizing metal ions.

In some embodiments, the association with the metal ions modulates a quantum yield of a photoluminescence exhibited by the self-assembled plurality of cyclic peptides, such that the quantum yield is higher. In some embodiments, the quantum yield is higher by at least 2 folds, compared to self-assembled plurality of cyclic peptides not in association with metal ions. In some embodiments, the quantum yield is higher than 5, or higher than 8, e.g., is from 5 to 20, or from 6 to 20, or from 10 to 20.

Applications and Uses:

The self-assembled structure or the light emitting system comprising or consisting of the same of the present embodiments optionally and preferably exhibits quantum confinement.

The term “quantum confinement,” as used herein refers to a phenomenon in which there are quantized energy levels in at least one dimension.

A structure (self-assembled structure of the present embodiments) exhibits quantum confinement when the positions of charge carriers (electrons or holes) in the structure are confined along at least one dimension. A structure in which the charge carriers are confined along one dimension but are free to move in the other two dimensions is referred to herein as a “two-dimensional quantum confinement structure,” since the structure allows free motion in two dimensions. A structure in which the charge carriers are confined along two dimensions but and are free to move only in one dimension is referred to herein as a “one-dimensional quantum confinement structure,” since the structure allows free motion in one dimension. A structure in which the charge carriers are confined along all three dimensions, namely a structure in which the charge carriers are localized, is referred to herein as a “zero-dimensional quantum confinement structure,” since the structure does not allow free motion.

A two-dimensional quantum confinement structure is interchangeably referred to herein as a quantum well structure, a one-dimensional quantum confinement structure is interchangeably referred to herein as a quantum wire structure, and a zero-dimensional quantum confinement structure is interchangeably referred to herein as a quantum dot structure.

In various exemplary embodiments of the invention the length L_{QC} of the smallest dimension along which a quantum confinement occurs is in the nanometer range, preferably below 3 nm or below 2 nm. In some embodiments of the present invention L_{QC} is in the sub-nanometer range (i.e., less than 1 nm), preferably less than 0.8 nm or less than 0.7 nm or less than 0.6 nm. This is an advantageous over traditional inorganic semiconductor quantum confinement structure which possess much higher quantum confinement lengths. L_{QC} is referred to as the quantum confinement length.

Quantum confinement can be verified by examining the optical properties of the structure. For quantum confinement structures, the optical properties are significantly different from other structures since the optical absorption coefficient is defined by density of states (DOS) of the charge carriers. For a structure which does not exhibits any quantum confinement the DOS is proportional to the square root of the energy. For a quantum confinement structure, the DOS is quantized. When the absorption spectrum of a structure has a step-like shape, the structure can be identified as a quantum well structure. A step-like shape of a spectrum is a widely used term in the scientific community and a person ordinarily skilled in the art of spectral analysis would recognize a spectrum having a step-like shape by observing a plot of the absorption coefficient as a function of the wavelength. Typically, but not exclusively, a step-like spectrum is characterized by a change of at least 10% in the absorption coefficient over a wavelength range of less than 10 nm.

When the absorption spectrum of a structure has a spike-like shape, the structure can be identified as a quantum dot structure. A spike-like shape of a spectrum is a widely used term in the scientific community and a person ordinarily skilled in the art of spectral analysis would recognize a spectrum having a spike-like shape by observing a plot of the absorption coefficient as a function of the wavelength. A spike-like spectrum is characterized by at least one peak in the absorption coefficient. Typically, but not exclusively, the width of a peak in a spike-like spectrum, as measured at half of the peak's height above the base of the peak, is less than 10 nm. The ability of the self-assembled structure of the present embodiments to exhibit quantum confinement makes it suitable for being incorporated in a variety of applications. The following lists a few, non-limiting, examples of applications that can incorporate the self-assembled structure according to some embodiments of the present invention:

The self-assembled structure of the present embodiments can be provided or be incorporated in a supramolecular photoluminescent material. Such a material can be incorporated in an optoelectronic device, such as, but not limited to, organic light emitting diodes (OLEDs), organic photovoltaic devices (OPVs), organic transistors, such as, but not limited to, organic field effect transistors (OFETs). Also contemplated are embodiments in which the self-assembled structure is incorporated in a nanoelectronic device, such as, but not limited to, a nano-transistor, e.g., a single electron transistor (SET), a nano-switch, a nano-sensor, and the like. In some embodiments of the present invention the example, the self-assembled structure is incorporated in a touch display screen.

The quantum confinement exhibited by the structure of the present embodiments can be utilized in quantum applications. For example, the self-assembled structure of the present embodiments can be incorporated in quantum communication, quantum computers, quantum information processing, quantum cryptography, calculations.

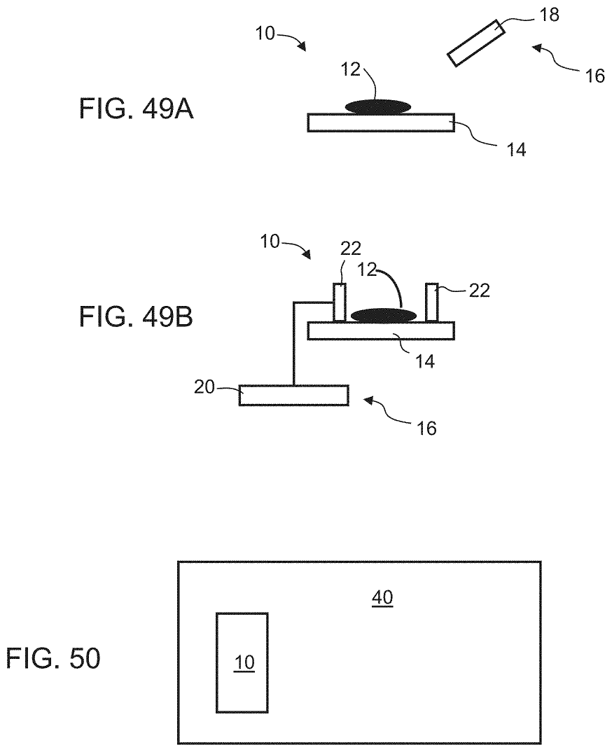

FIGS. 49A-B are schematic illustrations of a light emitting system 10, according to some embodiments of the present invention.

System 10 comprises a self-assembled structure 12 formed of a plurality of cyclic peptides in association with metal ions as described herein. Self-assembled structure 12 optionally and preferably exhibits quantum confinement. Optionally and preferably, but not necessarily, system 10 further comprises an excitation system 16 for exciting self-assembled structure 12 so as to emit light. In various exemplary embodiments of the invention self-assembled structure 12 emits the light at room temperature (e.g., at about 15-25° C.).

In various exemplary embodiments of the invention self-assembled structure 12 emits the light intrinsically, when exposed to UV-vis light.

The present embodiments contemplate several types of excitation systems 16 for exciting the self-assembled structure. Generally, the type of excitation system 16 is selected in accordance with the mechanism by which it is desired to have the light emitted from self-assembled structure 12.

FIG. 49A illustrates an embodiments of the invention in which excitation system 16 comprises a light source 18. In these embodiments, self-assembled structure 12 emits light via the photoluminescence effect. Light source 18 is preferably a monochromatic light source, e.g., a laser device.

FIG. 49B illustrates an embodiments of the invention in which excitation system 16 comprises or are connectable to a voltage source 20. In these embodiments, self-assembled structure 12 emits light via the electroluminescence effect. Source 20 can generate electric filed by means of electrodes 22. For clarity of presentation, voltage source 20 is illustrated as connected to only one of electrodes 22, but the skilled person would appreciated that more than one electrode can be connected to source 20. In some embodiments of the present invention, electrodes 22 injecting holes and electrons to self-assembled structure 12, in which case self-assembled structure 12 emits light via injection luminescence.

The difference between the embodiment in which self-assembled structure 12 emits light via electroluminescence and the embodiment in which self-assembled structure 12 emits light via injection luminescence is, inter alia, in the materials from which electrodes 22 are made and/or the voltage level of source 20. For generating light via injection luminescence, electrodes 22 are preferably made of materials having a different work function such that one electrode injects electrons and the other electrode injects holes (or equivalently receives electrons). In this embodiment the voltage source can be of relatively low voltage since it is not necessary for the generated electric field to be of high intensity. For generating light via electroluminescence, the effect is achieved primarily via application of sufficiently high electric field, in which case the electrodes can be made of the same material.

In various exemplary embodiments of the invention self-assembled structure 12 is deposited on a substrate 14 which can be made of any material, subjected to the luminescence effect by which the self-assembled structure emits the light.

For example, when self-assembled structure 12 emits light via the photoluminescence effect, substrate 14 can be made of any material, including inorganic materials such as glass or quartz or organic materials, typically polymeric materials. In this embodiment, substrate can be made of, or being coated by, a material which reflects the light generated by light source 18. Such construction can enhance the photo-excitation.

When self-assembled structure 12 emits light via the electroluminescence or injection luminescence effect, substrate 14 can be made of an electrically conductive or semi-conductive material in which case substrate 14 serves as one of the electrodes 22. Alternatively, electrodes 22 can be deposited directly on substrate 14, in which case substrate 14 is preferably made of an electrically isolating material. The conductive or semi-conductive substrate 14 can be organic or inorganic. In exemplary embodiments, substrate 14 is an organic material, e.g., a polymeric material such as PVK, PVP, and the like. In exemplary such embodiments the system is configured as OLED.

FIG. 50 is a schematic illustration of a utility system 40 according to various exemplary embodiments of the present invention. Utility system 40 incorporates system 10, and various other components depending on the application for which system 40 is employed. In some embodiments, utility system 40 is a laser system, in some embodiments, utility system 40 is display system, in some embodiments, utility system 40 is an optical communication system, in some embodiments, utility system 40 is an illumination system and in some embodiments, utility system 40 is an optical connector. Such utility systems are known in the art and the skilled person would know how to construct such system using light emitting system 10 of the present embodiments.

Since self-assembled structure 12 or light emitting system 10 exhibits quantum confinement, system 10 can be used for two-photon emission. Two-photon emission is a process in which quantum entangled photon pairs are emitted from the system. It is recognized that quantum confinement can be produced by quantum confinement structures. In these structures, pairs of entangled photons are emitted by single photon emission from pairs of entangled electrons. A two-photon emission system is advantageous since it possesses properties absent from other emission systems.

Following are representative examples for utility system 40 which examples are particularly suitable when system 10 is a two-photon emission system. It is noted, however, that many of these examples are also applicable when system 10 does not emit entangled photons.

In an aspect of some embodiments of the present invention utility system 40 is used for two-photon microscopy, two-photon spectroscopy or two-photon imaging. In these embodiments, the system emits two photons in the direction of a sample to induce two-photon absorption in the sample. Two-photon absorption is a process in which two distinct photons are absorbed by an ion or molecule, causing excitation from the ground state to a higher energy state to be achieved. The ion or molecule remains in the upper excited state for a short time, commonly known as the excited state lifetime, after which it relaxes back to the ground state, giving up the excess energy in the form of photons.

The use of the system of the present embodiments for microscopy and/or spectroscopy is advantageous because it allows a wider energy gap hence reduces or eliminates background photons emitted by other mechanism (e.g., infrared photons or photon emitted by thermal excitations). Thus, the two-photon emission system of the present embodiments increases signal to noise ratio.

When considering fluorescence, an important figure of merit is the quantum efficiency, defined to be the visible fluorescence intensity divided by the total input intensity. For display or spectroscopic applications based on two-photon induced fluorescence, the use of the two-photon system of the present embodiments facilitates dominance of radiative relaxation over non-radiative relaxation (phonons) hence increases the quantum efficiency.

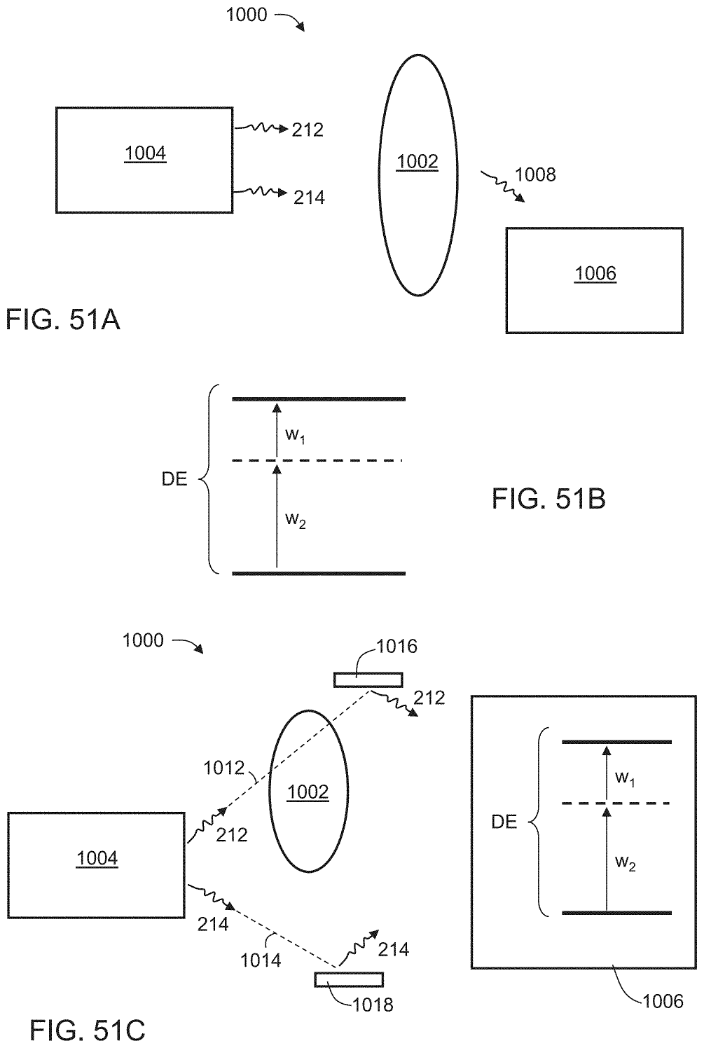

FIGS. 51A-B are schematic illustrations of a system 1000 for analyzing a target material 1002 by two photon absorption. System 1000 can be used for spectroscopy, microscopy and/or imaging of target material 1002. For example, when target material 1002 contains a fluorophore therein, system 1000 can be used for fluorescence spectroscopy. Representative examples of fluorophores suitable for the present embodiments include fluorophores which exhibit two-photon absorption cross-sections, such as the compositions described in U.S. Pat. No. 5,912,257, the contents of which are hereby incorporated by reference. Also contemplated are fluorophores which are normally excitable by a single short wavelength photon (e.g., ultraviolet photon). In this embodiment, the two-photon emission system emits two long wavelength photons (e.g., infrared photons) which can be simultaneously absorbed by such fluorophores.

System 1000 comprises a two-photon emission system 1004 which emits two photons 212 and 214 in the direction of material 1002 to induce two-photon absorption therein. System 1004 can be similar to system 10 described above. Preferably, device 1004 emits photons at predetermined frequencies at frequencies ω₁ and ω₂. The characteristic energy diagram is illustrated in FIG. 51B showing an energy gap ΔE=h(ω₁+ω₂)/2π. Thus photons generate excitation across ΔE. The value of the frequencies ω₁ and ω₂, is preferably selected such that ΔE is higher than the average energy of thermal and other background (e.g., infrared) photons. Once the material returns to its ground state, it emits radiation 1008 which can be detected by a detector 1006, as known in the art. System 1000 can employ any of the components of known systems for the analysis or imaging via two-photon absorption, see, e.g., U.S. Pat. Nos. 5,034,613, 6,020,591, 5,957,960, 6,267,913, 5,684,621, the contents of which are hereby incorporated by reference.

Reference is now made to FIG. 51C, which is a schematic illustration of system 1000 in an embodiment in which the detection is based on two-photon absorption. In this embodiment, the optical path 1012 of photon 212 can be arranged to pass through material 1002 and the optical path 1014 of photon 214 can be arranged to bypass material 1002. Both optical paths 1012 and 1014 terminate as detector 1006. Thus, photon 212 can serve as a signal photon and photon 214 can serve as an idler photon.

The wavelength of photon 212 is preferably selected to allow photon 212 to excite the molecules in material 1002. For example, the wavelength of photon 212 can be selected to match the vibrational or rotational resonances of the molecules in the material. In biological materials, such resonances are typically in the mid infrared or far infrared. For example, most of the absorption spectra of organic compounds are generated by the vibrational overtones or the combination bands of the fundamentals of O—H, C—H, N—H, and C— transitions. Thus, for biological materials, photon 212 can be a mid-infrared photon or a far infrared photon. Also contemplated are embodiments in which photon 212 is a near infrared photon which can be suitable for molecular overtone (harmonic) and combination vibrations. The use of other wavelengths (e.g., visible photons) is not excluded from the scope of the present invention.

Optical paths 1012 and 1014 can be established via an arrangement of optical elements 1016 and 1018 such as, but not limited to, mirrors, lenses, prisms, gratings, holographic elements, graded-index optical elements, optical fibers, or other similar beam-directing mechanisms.

When signal photon 212 passes through the material, it can be either absorbed by the material giving rise to a resonance in one of the molecules or continue to propagate therethrough, with or without experiencing scattering events. If signal photon 212 is not absorbed it can continue along path 1012 to detector 1006. Preferably optical paths 1012 and 1014 are of the same lengths such that when signal photon 212 successfully arrives at detector 1006 it arrives simultaneously with idler photon 214.

Detector 1006 is preferably characterized by a detection threshold which equals the sum of energies of photons 212 and 214. This can be achieved using a semiconductor detector having a sufficiently wide bandgap to allow two-photon absorption. For example, detector 1006 can be an Si detector.

Having a wide bandgap, detector 1006 does not provide a detection signal when only idler photon 214 arrives. Additionally, the wide bandgap prevents or reduces triggering of detector 1006 by noise, such as infrared background photons because the energy of such photons is lower than the detection threshold and further because triggering caused by simultaneous arrival of two background photons is extremely rare due to the random nature of the background photons.

Thus, detector 1006 provides indication of simultaneous arrival of the signal-idler photons pair, in a substantially noise-free manner. Such indication can provide information regarding material 1002 by means of transmission spectroscopy because the resonances appear as dips in the spectrum on the detector output. System 1000 can also operate according to similar principles in reflectance spectroscopy.

In an aspect of some embodiments of the present invention utility system 40 is used for communication applications. Since the light emitting system of the present embodiments typically emits two-photons simultaneously, the existence of one photon is an indication of the existence of another photon. Thus, a communication system incorporating the device of the present embodiments can use one photon as a signal and the other photon as an idler. More specifically, such communication system can transmit one photon to a distant location and use the other photon as an indication that a transmission is being made.

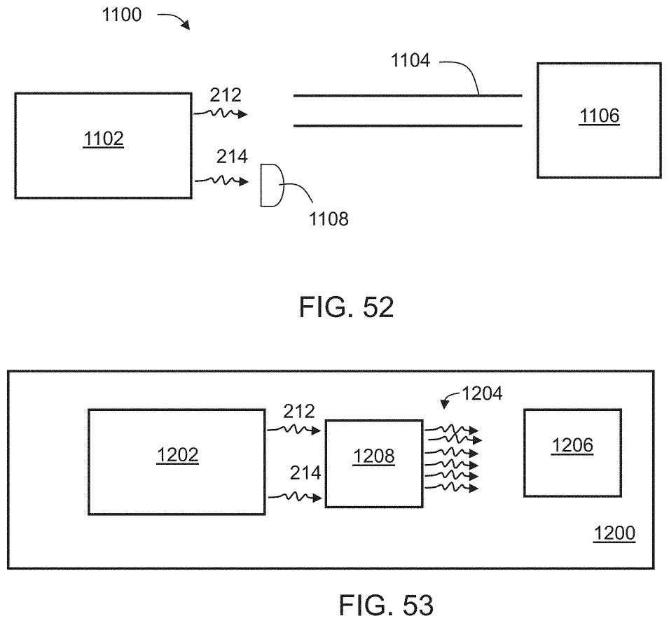

FIG. 52 is a schematic illustration of a communication system 1100 according to various exemplary embodiments of the present invention. System 1100 comprises a two-photon emission system 1102 which emits two photons 212 and 214. System 1102 can be similar to system 10 described above. Preferably, system 1102 emits photons at predetermined frequencies at frequencies ω₁ and ω₂. One photon (photon 212 in the present example) serves as a signal as is being transmitted over a communication channel 1104 such as an optical fiber or free air, while the other photon (photon 214 in the present example) serves as an idler and being detected by a detector for indicating that the signal has been transmitted.

Such communication system can be used for quantum cryptography and quantum teleportation.

Quantum cryptography provides security by means of physical phenomenon by the uncertainty principle of Heisenberg in the quantum theory. According to the uncertainty principle, the state of quantum will be changed once it is observed, wiretapping (observation) of communication will be inevitably detectable. This allows to take measures against the wiretapping, such as shutting down the communication upon the detection of wiretapping. Thus, quantum cryptography makes undetectable wiretapping impossible physically. Moreover, the uncertainty principle explains that it is impossible to replicate particles.

Quantum teleportation is a technique to transfer quantum information (“qubits”) from one place where the photons exist to another place.

A qubit is a quantum bit, the counterpart in quantum communication and computing to the binary digit or bit of classical communication and computing. Just as a bit is the basic unit of information in a classical signal, a qubit is the basic unit of information in a quantum signal. A qubit is conventionally a system having two degenerate (e.g., of equal energy) quantum states, wherein the quantum state of the qubit can be in a superposition of the two degenerate states. The two degenerate states are also referred to as basis states, and typically denoted I0> and I1>. The qubit can be in any superposition of these two degenerate states, making it fundamentally different from an ordinary digital bit.

Quantum teleportation can be used to transmit quantum information in the absence of a quantum communications channel linking the sender of the quantum information to the recipient of the quantum information. Suppose, for example, that a sender, Bob, receives a qubit α|0>+βI1> where and α and β are parameters on a unit circle. Bob needs to transmit to a receiver, Alice, but he does not know the value of the parameters and he can only transmit classical information over to Alice. According to the laws of quantum teleportation Bob can transmit information over a classical channel, provided Bob and Alice agree in advance to share a Bell state generated by an entangled state source. Such entangled state source can be the two-photon emission system of the present embodiments.

Thus, the system of the present embodiments can emit photons in a quantum entangled state hence be used in quantum cryptography and quantum teleportation.

In an aspect of some embodiments of the present invention utility system 40 is used as a component in a quantum computer.

Quantum computing generally involves initializing the states of several entangled qubits, allowing these states to evolve, and reading out the states of the qubits after the evolution. N entangled qubits can define an initial state that is a combination of 2^{N} classical states. This initial state undergoes an evolution, governed by the interactions that the qubits have among themselves and with external influences, providing quantum mechanical operations that have no analogy with classical computing. The evolution of the states of N qubits defines a calculation or, in effect, 2^{N} simultaneous classical calculations (e.g., conventional calculations as in those performed using a conventional computer). Reading out the states of the qubits after evolution completely determines the results of the calculations. For example, when there are two entangled qubits, 2²=4 simultaneous classical calculations can be performed. Taken together, quantum superposition and entanglement create an enormously enhanced computing power. Where a 2-bit register in an ordinary computer can store only one of four binary configurations (00, 01, 10, or 11) at any given time, a 2-qubit register in a quantum computer can store all four numbers simultaneously, because each qubit represents two values. If more qubits are entangled, the increased capacity is expanded exponentially.

FIG. 53 is a schematic illustration of a quantum computer system 1200 according to various exemplary embodiments of the present invention. System 1200 comprises a two-photon emission system 1202 which emits two photons 212 and 214, as described above. In this embodiment, photons 212 and 214 are in entangled state. System 1202 can be similar to system 10 described above. System 1200 further comprises a calculation unit 1206 which uses the photons as entangled qubits and perform calculations as known in the art (see, e.g., U.S. Pat. No. 6,605,822, the contents of which are hereby incorporated by reference). In various exemplary embodiments of the invention system 1200 comprises an optical mechanism 1208 for the generation of more than two entangled photons. For example, such mechanism can receive photons 212 and 214 emitted by system 1202, generate by reflection, refraction or diffraction two or more photons from each photon, so as to produce a plurality of entangled photons 1204.

Also contemplated are applications in which system 40 is used as an optical amplifier, in which the energy spectrum emitted by the two-photon is sufficiently broad. The use of the two-photon emission system of the present embodiments as an optical amplifier is advantageous because the gain in two-photon amplifier, in contrast to conventional single photon lasers, is nonlinear, depending on the amplitude of the light wave. Such two-photon amplifier can also be used for pulse generation. Since the length of the pulse is a decreasing function of the gain bandwidth of the amplifier, the broad spectrum of the two-photon system of the present embodiments facilitate generation of very short pulses.

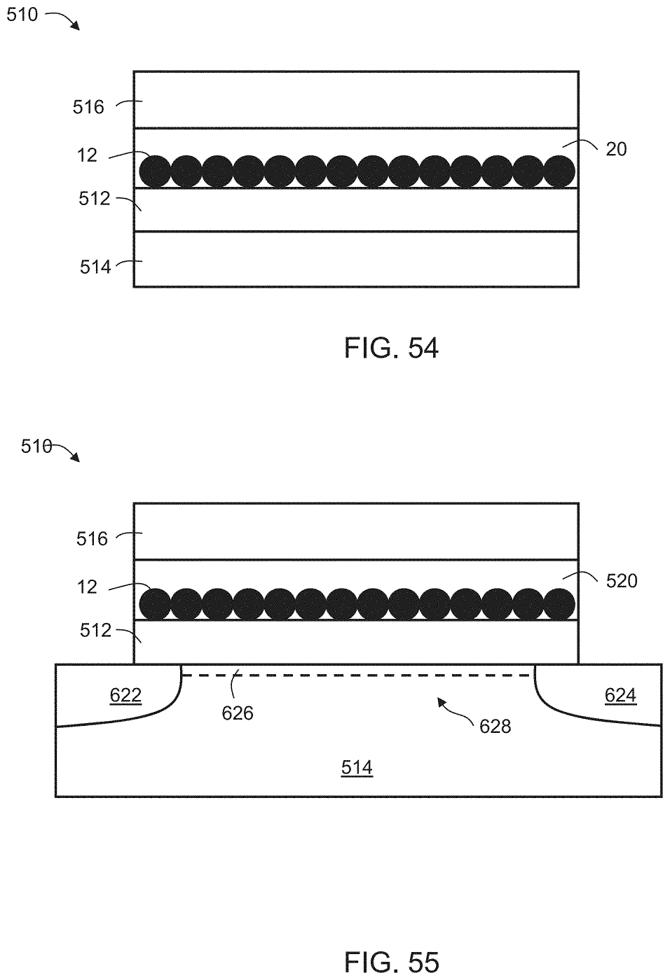

FIG. 54 illustrates a memory system 510, according to some embodiments of the present invention. System 510 comprises a memory layer 512 between a first layer 514 and a second layer 516, wherein first 514 and second 516 layers are configured to apply an electrical bias to memory layer 512. In various exemplary embodiments of the invention memory layer 512 comprises self-assembled structure 12 as described herein.

System 510 can have more than one operation principle. In some embodiments, voltage is applied to system 510, preferably between layers 514 and 516, and memory layer 520 shows bistable resistance values, therefore realizing desired memory properties. Specifically, depending on the density of states of structure 11, a tunneling current through memory layer 512 exhibits a bistable current for at least some values of the applied voltage.

FIG. 55 is a schematic illustration of system 510 in embodiments of the invention in which system 510 operates according to the operation principle of a transistor. In these embodiments first layer 514 comprises a source region 622 and a drain region 624 being separated laterally over layer 514 to define a channel region 628 between regions 622 and 624. Second layer 516 serves as or comprises a gate electrode.

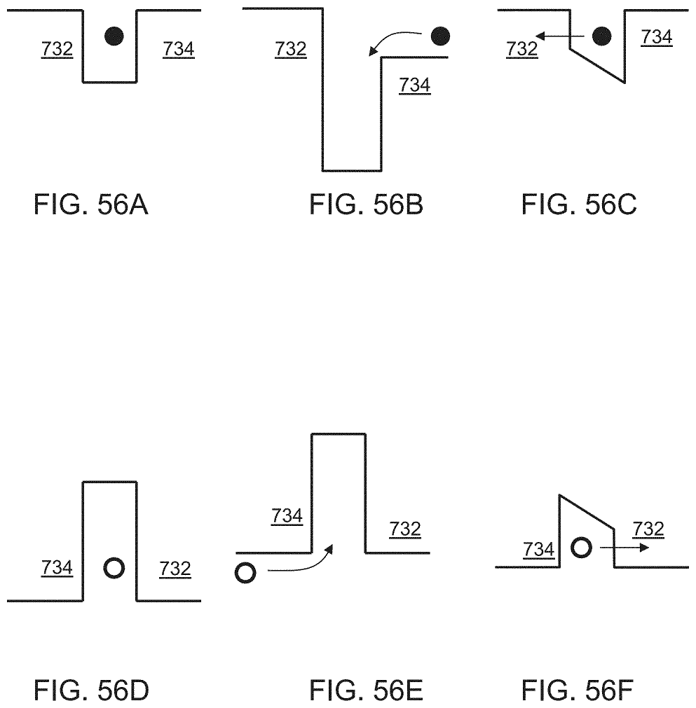

FIGS. 56A-F are schematic illustrations of exemplary characteristic bandgap diagrams of the self-assembled structure of the present embodiments. The illustrated bandgap is particularly useful when the self-assembled structure is incorporated in a memory system, such as, but not limited to, system 510.

FIGS. 56A-C schematically illustrate bandgap diagrams when the majority charge carriers in layer 514 are electrons (shown as full circles), and FIGS. 56D-F schematically illustrate bandgap diagrams when the majority charge carriers in layer 514 are holes (shown as empty circles).

When charge storage is required, the binding potential of the charge carrier (electrons or holes) in the self-assembled structure represents an emission barrier 732. Such storage can represent a logic state “1” (FIGS. 56A and 56D). When it is desired to maintain a logic state “0” a capture barrier 734 can be formed by band-bending using the gate electrode.

To write a logic state “1” into memory layer 512, a forward bias can be applied to gate electrode 516. The forward bias is preferably selected to reduce or eliminate the capture barrier formed by the band-bending, thus allowing fast write time (FIGS. 56B and 56E).

To erase the information from memory layer 512, the electric field at the position of the self-assembled structure can be increased by applying a reverse bias at the gate electrode 516 to effect emission by tunneling (FIGS. 56C and 56F).

To read information from memory layer 512 a two-dimensional charge carrier channel 626 (namely a two-dimensional electron gas or a two-dimensional hole gas) is preferably formed between source 622 and drain 624. The charge carriers stored in the self-assembled structure affect the charge density and the mobility in charge carrier channel 626. Thus, the charge density and/or mobility in the channel 626 is indicative of existence of charge carriers in the self-assembled structure. Thus, by measuring the charge density and/or mobility between source 622 and drain 624 (for example, by evaluating the electrical resistance or conductance of the layer carrying gas 626), the presence, level or absence of charge carriers in self-assembled structure 12 is determined.

The layers and regions of system 510 can be of any type known in the art of memory systems. For example, layer 514 can be a p-type semiconductor, e,g, (p-doped silicon), layers 512 can be made of silicon oxide on which structure 11 is deposited, additional layer 520 can be made of silicon oxide, and layer 16 can be made of polycrystalline silicon. Source region 622 and drain region 624 can be made, for example, from N+ silicon, as known in the art.

It is to be noted that the shape of self-assembled structure 12 in any of the respective figures is for illustrative purposes and should not be regarded as indicative or limiting in any way.

In exemplary embodiments, self-assembled structure 12 is deposited on the respective substrate as a thin layer of nanofibers or a plurality of quantum dots, depending on the shape and dimensions of the self-assembled structure.

The self-assembled structure can be used in the biomedicine field. For example, the self-assembled structure can be used as an imaging agent (intracellular or extracellular) for imaging, for example, for drug delivery and/or monitoring, cancer therapy, visual detection of metabolic activities, and the like.

For example, the self-assembled structure or a light-emitting system comprising or consisting of the same, can be used in combined therapeutic and diagnostic modalities on the same delivery system, such as in the field of a theranostic (therapy and diagnostic) nanomedicine. Information obtained from theranostic nanomedicine is exploited for fine tuning the therapeutic dose, while monitoring the progression of the diseased tissue, treatment efficacy and delivery kinetics. Such an approach enhances early diagnosis and treatment and may decrease drugs under-or over-dosing, resulting in a more personalized treatment.

Since the signal from fluorescent probes in vivo is impeded by the emitted fluorescence from tissues and biomolecules (e.g., water, melanin, proteins and hemoglobin), which absorb photons in the wavelengths range of 200-650 nm (i.e., low signal-to-noise ratio), intravital imaging at higher spectral ranges is desirable. At the higher range, auto-fluorescence is minimal and scattering of light is reduced, enabling deep tissue penetration and facilitating non-invasive monitoring.

The self-assembled structures of the present embodiments, be being capable of emitting light at higher wavelengths can thus serve as efficient imaging agents in diagnostic and theranostic applications.

According to an aspect of some embodiments of the present invention there is provided a light emitting system which comprises a self-assembled structure as described herein in any of the respective embodiments and therapeutically active agent being in association with the self-assembled structure. The therapeutically active agent can form a part of the self-assembled structure or can be encapsulated, encaged, embedded, entrapped or absorbed in or on the structure.

According to an aspect of some embodiments of the present invention there is provided a pharmaceutical composition that comprises such a system.

The pharmaceutical composition can comprise the system, optionally in combination with a pharmaceutically acceptable carrier.

Herein, the phrase “pharmaceutically acceptable carrier” refers to a carrier or a diluent that does not cause significant irritation to an organism and does not inhibit the distribution, therapeutic properties or otherwise does not abrogate the biological activity and properties of the administered or applied structure as described herein and/or therapeutically active agent as described herein.

Herein the term “excipient” refers to an inert substance added to a pharmaceutical composition to further facilitate administration or application of a drug.

Techniques for formulation and administration of drugs may be found in “Remington's Pharmaceutical Sciences” Mack Publishing Co., Easton, Pa., latest edition, which is incorporated herein by reference.

Pharmaceutical compositions for use in accordance with the present invention thus may be formulated in conventional manner using one or more pharmaceutically acceptable carriers comprising excipients and auxiliaries, which facilitate processing of the active ingredient(s) into preparations which can be used pharmaceutically. Proper formulation is dependent upon the route of administration chosen. The dosage may vary depending upon the dosage form employed and the route of administration utilized. The exact formulation, route of administration and dosage can be chosen by the individual physician in view of the patient's condition (see e.g., Fingl et al., 1975, in “The Pharmacological Basis of Therapeutics”, Ch. 1 p.1).

The pharmaceutical composition may be formulated for administration in either one or more of routes depending on whether local or systemic treatment or administration is of choice, and on the area to be treated. Administration may be done orally, by inhalation, or parenterally, for example by intravenous drip or intraperitoneal, subcutaneous, intramuscular or intravenous injection, or topically (including transdermally, ophtalmically, vaginally, rectally, intranasally).

The amount of a composition to be administered or otherwise applied will, of course, be dependent on the subject being treated, the severity of the affliction, the manner of administration, the judgment of the prescribing physician, etc.

Compositions of the present invention may, if desired, be presented in a pack or dispenser device, such as an FDA (the U.S. Food and Drug Administration) approved kit, which may contain one or more unit dosage forms containing the active ingredient. The pack may, for example, comprise metal or plastic foil, such as, but not limited to a blister pack or a pressurized container (for inhalation). The pack or dispenser device may be accompanied by instructions for administration. The pack or dispenser may also be accompanied by a notice associated with the container in a form prescribed by a governmental agency regulating the manufacture, use or sale of pharmaceuticals, which notice is reflective of approval by the agency of the form of the compositions for human or veterinary administration. Such notice, for example, may be of labeling approved by the U.S. Food and Drug Administration for prescription drugs or of an approved product insert. Compositions comprising active ingredient(s) according to embodiments of the invention formulated in a compatible pharmaceutical carrier may also be prepared, placed in an appropriate container, and labeled for treatment of a particular medical condition, disease or disorder, as is detailed herein.

Such a system or a composition comprising same can be used in a theranostic approach as described herein, for delivering and monitoring the activity of the therapeutically active agent. Such a system can be for use in a method of treating a subject having a medical condition treatable by the therapeutically active agent and/or for monitoring the treatment of the medical condition, as described herein.

Any therapeutically active agents and corresponding medical conditions can be used according to these embodiments. Examples include anti-cancer agents for treating cancer, antibiotics for treating bacterial infections, anti-inflammatory agents for treating inflammation, and the like.

The light emitting system as described herein (without a therapeutically active agent) can alternatively be used in combination with a pharmaceutical composition that comprises a therapeutically active agent, by being administered to a subject concomitantly with the therapeutic composition for monitoring the delivery and/or efficacy of the therapeutically active agent.

Given the essential roles metal ions play in peptide self-assembly, the quantum-confined self-assembled structures of the present embodiments can be used as fluorescent markers to detect the dynamics of aggregations processes, such as amyloid formation by proteins and polypeptides. This allows visual detection of metabolic activities, by acting by interaction with metal ions. Herein, the hierarchical aggregation of proteins and polypeptides is detected, and the prospective physicochemical, physiological and pathological features is probed. This allows unlocking the secrets of the optical behaviors of peptides/proteins self-assemblies in neuronal signaling and control, and clarifying the pathogenesis underlying neural degenerations and exploring methodologies to inhibit the process.

The nanoscale sizes, quantum confined properties and intrinsic biocompatibility allow the self-assembled structures to be implanted into neuronal cells, in order to investigate the interface between the structures and neurons. Therefore, the metabolic procedures of the assemblies in neural cells and their response and influence on neuronal activities (such as synaptic activities and signal transduction) can be studied by tracking their photoluminescent signals. This can lay the basis for future diagnosis and treatment of sensory functions, which will be explored to facilitate the recovery of sensory functions in defective neuronal systems. In this scenario, with the advantages of high sensitivity to impulses, outstanding optical or electrical properties, the bio-inspired self-organizations may bring relief to the sufferers and their caretakers.

As used herein the term “about” refers to ±10% or ±5%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

As used herein, the term “alkyl” refers to a saturated aliphatic hydrocarbon including straight chain and branched chain groups. Preferably, the alkyl group has 1 to 20 carbon atoms. The alkyl group may be substituted or unsubstituted. When substituted, the substituent group can be, for example, trihaloalkyl, alkenyl, alkynyl, cycloalkyl, aryl, heteroaryl, heteroalicyclic, halo, nitro, azo, hydroxy, alkoxy, thiohydroxy, thioalkoxy, cyano, and amine.

A “cycloalkyl” group refers to an all-carbon monocyclic or fused ring (i.e., rings which share an adjacent pair of carbon atoms) group wherein one of more of the rings does not have a completely conjugated pi-electron system. Examples, without limitation, of cycloalkyl groups are cyclopropane, cyclobutane, cyclopentane, cyclopentene, cyclohexane, cyclohexadiene, cycloheptane, cycloheptatriene, and adamantane. A cycloalkyl group may be substituted or unsubstituted. When substituted, the substituent group can be, for example, alkyl, trihaloalkyl, alkenyl, alkynyl, cycloalkyl, aryl, heteroaryl, heteroalicyclic, halo, nitro, azo, hydroxy, alkoxy, thiohydroxy, thioalkoxy, cyano, and amine.

An “alkenyl” group refers to an alkyl group which consists of at least two carbon atoms and at least one carbon-carbon double bond.

An “alkynyl” group refers to an alkyl group which consists of at least two carbon atoms and at least one carbon-carbon triple bond.

An “aryl” group refers to an all-carbon monocyclic or fused-ring polycyclic (i.e., rings which share adjacent pairs of carbon atoms) groups having a completely conjugated pi-electron system. Examples, without limitation, of aryl groups are phenyl, naphthalenyl and anthracenyl. The aryl group may be substituted or unsubstituted. When substituted, the substituent group can be, for example, alkyl, trihaloalkyl, alkenyl, alkynyl, cycloalkyl, aryl, heteroaryl, heteroalicyclic, halo, nitro, azo, hydroxy, alkoxy, thiohydroxy, thioalkoxy, cyano, and amine.

A “heteroaryl” group refers to a monocyclic or fused ring (i.e., rings which share an adjacent pair of atoms) group having in the ring(s) one or more atoms, such as, for example, nitrogen, oxygen and sulfur and, in addition, having a completely conjugated pi-electron system. Examples, without limitation, of heteroaryl groups include pyrrole, furane, thiophene, imidazole, oxazole, thiazole, pyrazole, pyridine, pyrimidine, quinoline, isoquinoline and purine. The heteroaryl group may be substituted or unsubstituted. When substituted, the substituent group can be, for example, alkyl, trihaloalkyl, alkenyl, alkynyl, cycloalkyl, aryl, heteroaryl, heteroalicyclic, halo, nitro, azo, hydroxy, alkoxy, thiohydroxy, thioalkoxy, cyano, and amine.

A “heteroalicyclic” group refers to a monocyclic or fused ring group having in the ring(s) one or more atoms such as nitrogen, oxygen and sulfur. The rings may also have one or more double bonds. However, the rings do not have a completely conjugated pi-electron system. The heteroalicyclic may be substituted or unsubstituted. When substituted, the substituted group can be, for example, lone pair electrons, alkyl, trihaloalkyl, alkenyl, alkynyl, cycloalkyl, aryl, heteroaryl, heteroalicyclic, halo, nitro, azo, hydroxy, alkoxy, thiohydroxy, thioalkoxy, cyano, and amine. Representative examples are piperidine, piperazine, tetrahydrofuran, tetrahydropyrane, morpholino and the like.

A “hydroxy” group refers to an —OH group.

A “thio”, “thiol” or “thiohydroxy” group refers to and —SH group.

An “azide” group refers to a —N═N=group.

An “alkoxy” group refers to both an —O-alkyl and an —O-cycloalkyl group, as defined herein.

An “aryloxy” group refers to both an —O-aryl and an —O-heteroaryl group, as defined herein. A “thiohydroxy” group refers to and —SH group.

A “thioalkoxy” group refers to both an —S-alkyl group, and an —S-cycloalkyl group, as defined herein.

A “thioaryloxy” group refers to both an —S-aryl and an —S-heteroaryl group, as defined herein.

A “halo” or “halide” group refers to fluorine, chlorine, bromine or iodine.

A “trihaloalkyl” group refers to an alkyl substituted by three halo groups, as defined herein. A representative example is trihalomethyl.

An “amino” group refers to an —NR′R″ group where R′ and R″ are hydrogen, alkyl, cycloalkyl or aryl.

A “nitro” group refers to an —NO₂ group.

A “cyano” group refers to a —C≡N group.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Materials and Experimental and Analytical Methods

Cells lines and cultures: The MCF-7 human breast cancer cell line was purchased from the American Type Culture Collection (ATCC). The B16-BL6 murine melanoma cell line was obtained from the National Cancer Institute-Central Repository. The human skin HaCaT keratinocyte cell line, a transformed human epidermal cell line, was obtained from the Germany Cancer Research Center.

MCF-7 cells were cultured in Eagle's Minimum Essential Medium supplemented with 10% FBS. B16-BL6 and HaCaT cells were cultured in Dulbecco's Modified Eagle's Medium supplemented with 10% FBS. All culture media contained 1% penicillin and cells were maintained at 37° C. in a humidified 5% CO2 incubator. Cells were periodically examined to verify the absence of mycoplasma contamination using the commercial detection kit (Lonza, Switzerland, LT07-703).

Mice and care: Athymic male NU/NU nude mice (6-week old) were purchased from Charles River (Wilmington, Mass., USA) and maintained on a 16:8 hours light-dark cycle. All procedures for animal use were approved by the Institutional Animal Care and Use Committee (IACUC) at The Ohio State University.

Materials: Aromatic cyclo-dipeptides were purchased from Bachem (Bubendorf, Switzerland), DGpeptides (Hangzhou, China) or GL Biochem (Shanghai, China), anhydrous zinc chloride (ZnCl₂), copper chloride dihydrate (CuCl₂.2H₂O) and anhydrous MeOH from Sigma Aldrich (Rehovot, Israel), DMSO from Sigma-Aldrich (St. Louis, Mo.). All materials were used as received without further purification. Water was processed using a Millipore purification system (Darmstadt, Germany) with a minimum resistivity of 18.2 MS/cm.

Sample preparation: Cyclo-dipeptides were added to anhydrous MeOH, MeOH solutions of metal salts (e.g., ZnCl₂ or CuCl₂), DMSO, or DMSO solutions of metal salts (e.g., ZnCl₂), to final concentrations of 5.0 mM cyclo-dipeptides and 10.0 mM metal salts. To dissolve the peptides, the solutions were incubated in a water bath at 70 or 80° C. for 5 minutes, after which most of the solutions became transparent.

UV-Vis spectra: UV-Vis spectra between 200 to 800 nm were recorded on an Agilent Cary 100 UV-Vis spectrophotometer with a quartz cuvette of 1 mm path length.

Fluorescence:

UV-Visible fluorescence characterization: 600 μL sample solution was pipetted into a 1.0 cm path-length quartz cuvette, and the spectrum was collected using a FluoroMax-4 Spectrofluorometer (Horiba Jobin Yvon, Kyoto, Japan) at ambient temperature. For molecular fluorescence of some experiments, the emission wavelength was set at 350 nm with a slit of 3 nm, and the excitation wavelength was set at 200-330 nm with a slit of 3 nm. For self-assembly fluorescence, the excitation wavelength was set at 370 nm with a slit of 3 nm, and the emission wavelength was set at 380-700 nm with a slit of 3 nm. For excitation-dependent maximal emission evolution experiment, the excitation wavelengths were set at 300-450 nm with a slit of 3 nm, and the emission wavelengths were set at 380-600 nm with a slit of 3 nm. According to the samples, anhydrous MeOH or MeOH solution of ZnCl₂ (CuCl₂) was used as background and subtracted. At least five measurements were performed and averaged for accuracy. Emission and excitation wavelengths, slit dimensions and background solutions were adjusted per the measured sample and are as indicated below.

NIR fluorescence characterization: Cary Eclipse Fluorescence Spectrophotometer (Agilent Technologies) was used for measuring fluorescence excitation and emission spectra, with both slits width set at 5 nm. Three measurements were performed and averaged for accuracy.

Fluorescent decay measurement (lifetime): 600 μL sample solution was pipetted into a 1.0 cm path-length quartz cuvette, and the spectrum was collected using a FluoroMax-4 Spectrofluorometer (Horiba Jobin Yvon, Kyoto, Japan) equipped with a NanoLED laser excitation source at ambient temperature. The wavelength was set as the maximum excitation and emission of the samples, and a LUDOX sample (silica beads, 2 μm) was used as the prompt. The lifetime was determined by fitting the fluorescent decay data from the DAS6 Analysis software (Horiba Jobin Yvon, Kyoto, Japan). Three measurements were performed and averaged for accuracy.

Fluorescence photobleaching: The kinetic measurements of Cy5.5 (excitation: 650 nm, emission: 720 nm), ICG (excitation: 785 nm, emission: 826 nm) and cyclo-WW+Zn(II) in DMSO (excitation: 550/680/770 nm, emission: 615/712/817 nm) were conducted using a Cary Eclipse Fluorescence Spectrophotometer (Agilent Technologies). The fluorescence emission data was collected at 80 points per second for 10 minutes. The excitation and emission slit widths were each set at 5 nm. At least three measurements were performed and averaged for accuracy.

Fluorescence photostability: Fluorescence emission spectra were measured at different time points (1 day, 7 days and 14 days). The excitation and emission slit widths were each set at 5 nm. Three measurements were performed and averaged for accuracy.

UV-Vis absorption: For cyclo-dipeptides solutions absorption, 45 μL of the sample solution was pipetted into a 96-well UV-Star UV transparent plate (Greiner BioOne, Frickenhausen, Germany), and the UV-Vis absorbance was recorded using a Biotek Synergy HT plate reader (Biotek, Winooski, Vt., USA), with a normal reading speed and calibration before reading. Anhydrous MeOH was used as background and subtracted. Three measurements were performed and averaged for accuracy.

QD radius calculation: The QD (quantum dot) radius was calculated based on the model of organic QDs³⁴, as shown in the following Equation (1):

$$R = π {r}_{B}^{0} \sqrt{\frac{{m}_{0} / M}{\frac{μ}{{m}_{0} {ɛ}_{∞}^{2}} - \frac{{E}_{ex}^{QD}}{{R}_{y}}}} \qquad ( 1 )$$

Where r_{B}⁰=h²/m₀e²=0.53 Å is the Bohr radius of the hydrogen atom; m₀ is the free electron mass (9.11×10⁻³¹ kg); R_{y}=m₀e⁴/2 h²=13.56 eV is the Rydberg constant; M=mₑ+mₕ is the translation mass of the exciton (mₑ and mₕ are the effective mass of electron and hole, respectively); μ=mₑmₕ (mₑ+mₕ) is the reduced exciton mass; ε_{∞} is the high-frequency dielectric constant of the QD; Eₑₓ^{QD} is the exciton binding energy.

Since obtaining the accurate reflective index of peptide QDs is technically challenging, the refractive index of the analogous benzene crystal, namely n=1.5, was used to calculate ε_{∞}. This defines ε_{∞}=n²=2.25.

The optical absorption starts from λᵢₒₙ 250 nm (hωᵢₑₙ=4.96 eV) (FIG. 2B), corresponding to the breaking of the binding exciton state. The value of hωᵢₒₙ corresponds to the QD energy gap. The difference between hωᵢₑₙ and the phononless line hω_{g}⁰=4.59 eV equals 0.37 eV, representing the exciton binding energy, Eₑₓ^{QD} of the QD.

The effective masses of electrons and holes are almost identical and close to 0.5m₀. Consequently, for μ=0.5mₑ=0.25m₀ and M=M₀, the QD diameter of the cyclo-dipeptides was calculated to be D≈2.24 nm, approximately the dimension of a dimer.

For Job Plot analysis of cyclo-WW+Zn(II), a fixed total concentration of 15.0 mM was used, with the following molar proportions (corresponding concentrations) of cyclo-WW: 0.0 (0.0 mM), 0.1 (1.5 mM), 0.2 (3.0 mM), 0.3 (4.5 mM), 0.4 (6.0 mM), 0.5 (7.5 mM), 0.6 (9.0 mM), 0.7 (10.5 mM), 0.8 (12.0 mM), 0.9 (13.5 mM), 1.0 (15.0 mM). 1 mL sample of each solution was pipetted into a 1.0 cm path-length quartz cuvette, and a T60 visible spectrophotometer (PG Instruments, Leicestershire, United Kingdom) was used for spectra collection with a fixed spectral bandwidth of 2 nm and a 200-800 nm wavelength range. Anhydrous MeOH solutions of ZnCl₂ at the corresponding concentrations were used as background and subtracted. The absorbance at 515 nm was extracted to generate the Job Plot vs. molar proportions of cyclo-WW.

Theoretical calculations of molecular orbital amplitudes and energy levels: Density functional theory calculations were carried out based on the self-consistent solution of Kohn-Sham function and the projector augmented wave pseudopotential as implemented in Vienna Ab-initio Simulation Package (VASP). The exchange-correlation potential is in the form of Perdew-Burke-Ernerhof (PBE) with generalized gradient approximation (GGA). For the structural relaxation, the energy convergence threshold was set to 10⁻⁵ eV and the residual force on each atom was less than 0.03 eV Å⁻¹. The cutoff energy for the plane-wave basis was set to 500 eV. To eliminate interaction between the molecule and its periodic images, a vacuum distance larger than 15 Å for each direction in the supercell geometry was used.

Nucleic magnetic resonance (NMR): Cyclo-WW or cyclo-WW+Zn(II) were dissolved in deuterated solvent with tetramethylsilane as the internal standard to prepare sample solutions with 5.0 mM dipeptide and 10.0 mM Zn(II). ¹H NMR spectra were recorded on a Bruker AV-400 NMR spectrometer with chemical shifts reported as ppm. The difference in the chemical shifts values before and after the addition of Zn (II) into the cyclo-WW solution (Δδ=(δ_{cyclo-WW})−δ(_{cyclo-WW+zn (II))}) were calculated in ppb and plotted as a function of amide, aromatic and aliphatic protons.

Mass spectrometry (MS): The MS experiment was performed using a LCMS Xevo-TQD system including an Acquity model UPLC and a triple quad mass spectrometer (Waters, Mass., USA). The positive electrospray ionization (ES+) channel was used for analysis.

Scanning electron microscopy (SEM): 20 μL solution samples were placed onto a clean glass slide and allowed to adsorb for a few seconds. After removing excessive liquid with filter paper, the slide was coated with Cr and observed under a JSM-6700 field emission scanning electron microscope (JEOL, Tokyo, Japan) operated at 10 kV.

Quantum yield (QY) measurement: 5 mL sample solution was pipetted into a 1.0 cm path-length quartz cuvette with a tube (Hellma, Müllheim, Germany), and the absolute quantum yield was measured on an absolute PL quantum yield spectrometer C11347 (Hamamatsu Photonics, Shizuoka, Japan) at ambient temperature. The relevant blanks, namely anhydrous MeOH, MeOH solutions of ZnCl₂ (CuCl₂), or DMSO solution of ZnCl₂, were used as background and subtracted. At least five measurements were performed and averaged for accuracy.

Dynamic light scattering (DLS): 850 μL of the sample solution was introduced into a DTS1070 folded capillary cell (Malvern, Worcestershire, U.K.), and the size was measured using a Zetasizer Nano ZS analyzer (Malvern Instruments, Malvern, UK) at 25.0° C. and a backscatter detector (173°). Three measurements were performed and averaged for accuracy.

Fourier transform infrared spectroscopy (FTIR): 750 μL of the sample solution was dropped onto polystyrene IR card (International Crystal Labs, Garfield, N.J., USA) and air drying. The FTIR spectra were recorded on a Nicolet 6700 FTIR spectrometer (Thermo Scientific, Waltham, Mass., USA), from 4000 to 400 cm⁻¹ at room temperature. 128 scans were collected with a spectral resolution of 4 cm⁻¹ in nitrogen atmosphere. Corresponding reference spectra (anhydrous MeOH or MeOH solution of ZnCl₂ (CuCl₂)) were recorded under identical conditions and subtracted.

Atomic force microscopy (AFM): 4 μL of sample solution was dropped onto a freshly cleaved mica surface and let it dry. The mica was then rinsed with water and purged dried gently with nitrogen. A topographic image was recorded under a NanoWizard 3 BioScience AFM (JPK, Berlin, Germany) in the tapping mode at ambient temperature, with 512×512 pixel resolution and a scanning speed of 1.0 Hz.

Transmission electron microscopy (TEM): A carbon-coated copper grid was placed on a 10 μL cyclo-dipeptide droplet for 1 minute and then blotted. Next, the washed grid was placed on a 10 μL droplet containing 4% (w/v) uranyl acetate solution for 1 min and then blotted. Samples were examined using Tecnai G2 Spirit TEM (FEI) at 80 kV.

X-ray diffraction (XRD): Spectra were recorded using a Bruker D8 Advance X-ray powder diffractometer (Bruker) at room temperature with a scan range 2θ of 5-45° and a count of two seconds. Cyclo-WW+Zn(II) nanospheres powder was placed on the standard flat sample reflection holder. Data collection and analysis was performed using the MDI Jade software.

Fabrication and characterization of LEDs: Commercially available InGaN chips were used at the bottom of the LED base. For the preparation of the color conversion layer, the cyclo-dipeptide+Zn(II) MeOH solution was purged dry using ultrapure nitrogen, and then mixed with PDMS at a mass ratio of 1:1. The mixtures were applied on InGaN chips and after curing at 80° C. for 1 hour, the LEDs peptides phosphors were obtained.

Cytotoxicity: HaCaT, B16-BL6 and MCF-7 cells were seeded in a 96-well plate with 100 μL of culture medium per well and incubated at 37° C. in 5% CO2 for 12 h to allow the cells to adhere to the surface. The medium was then replaced with a medium containing cyclo-WW+Zn(II) nanospheres at two different concentrations (62.5, 125.0 nM in cell culture medium), or with naïve medium as a control. Cells were incubated for 24 h before determining cell viability using the CCK-8 assay (Dojindo Molecular Technologies), according to the manufacturer's instructions. The absorbance at 490 nm was determined using Opsys microplate reader (Dynex Technologies, Chantilly, Va.).

In vivo NIR imaging: Cyclo-WW+Zn(II) nanospheres (50 μL, 2.7 mM diluted in water) were administered into nude mice by subcutaneous injection. Whole body NIR fluorescence imaging was conducted with the mice anesthetized (2.5% isoflurane in oxygen flow, 1.5 L min⁻¹) immediately following the injection using an IVIS Spectrum Imaging System (PerkinElmer). All images were taken using emission filters designed for DsRed (575-650 nm), Cy5.5 (695-770 nm) and ICG (810-875 nm), with excitation wavelengths of 535, 640 and 745 nm, respectively. The fluorescent light emitted from the mice was detected by a CCD camera. Data acquisition and analysis was performed using the Living Image 4.2.1 software.

### Example 1

### Dimeric QDs as Building Blocks for Self-Assembly

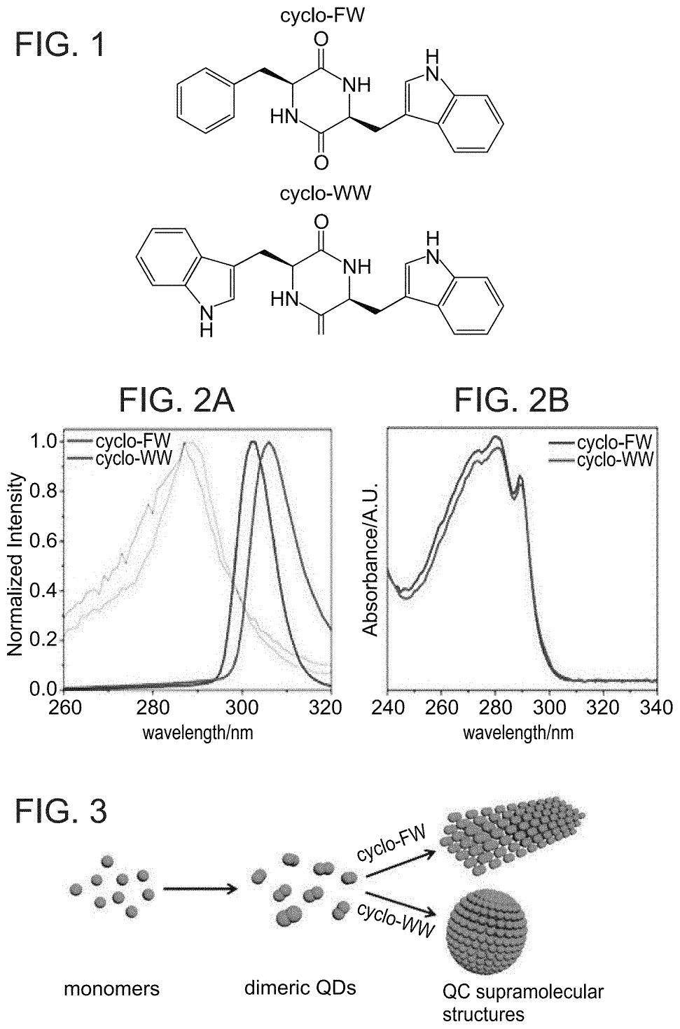

The photoluminescent properties of two tryptophan (W)-containing aromatic cyclo-dipeptides [Gazit, E. Peptide nanostructures: aromatic dipeptides light up. Nature Nanotechnol. 11, 309-310 (2016)], cyclo-phenylalanine-tryptophan (cyclo-FW) and cyclo-WW (shown in FIG. 1), both dissolved in methanol (MeOH), were characterized.

Fluorescent characterization demonstrated red shifts of the molecular excitation to 305 nm, compared to 285 nm for the monomers (FIG. 2A), indicating that the cyclo-dipeptides indeed self-assembled, which was further confirmed by dynamic light scattering (DLS) detections (FIG. 5).

UV-Vis absorption spectra revealed that the cyclo-dipeptides had spike-like absorbance, showing three peaks at 273 nm, 280 nm and 289 nm (FIG. 2B), characteristic of the formation of QD structures.

The diameter of the QDs was calculated (as described hereinabove) to be about 2.24 nm, around two-fold of the dimension of the monomer.

In addition, mass spectrometry (MS) analysis detected the dimeric molecular weights (MW), along with the corresponding monomeric molecular mass indicating the presence of dimers (data not shown). Therefore, it can be concluded that the cyclo-dipeptide monomers first formed dimers, which behaved as QDs and served as the fundamental building blocks to further organize into supramolecular structures, as represented schematically in FIG. 3.

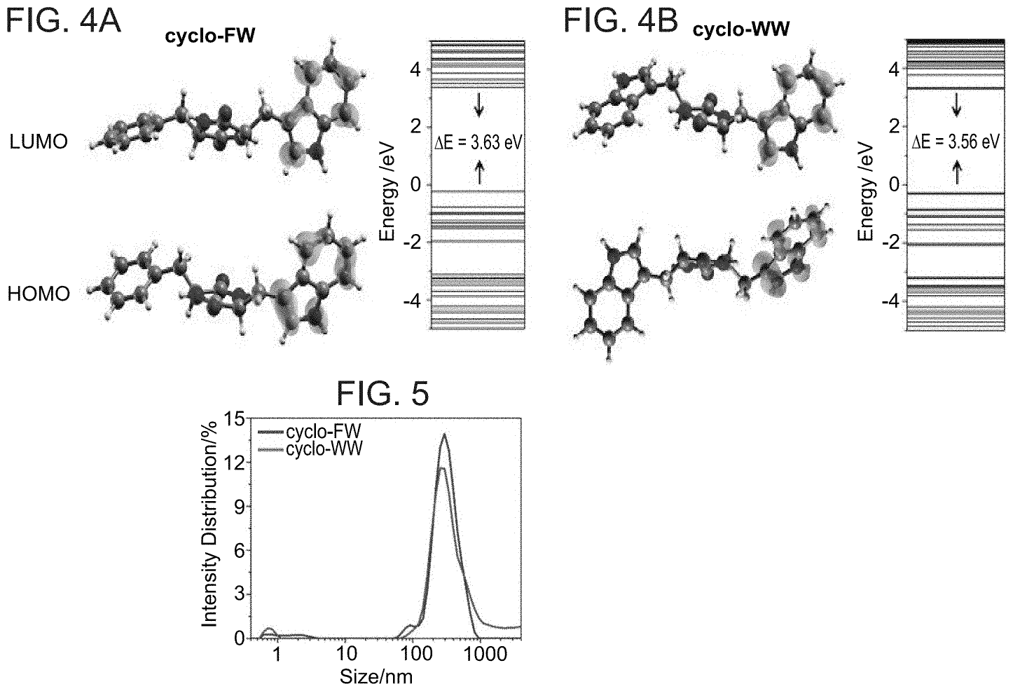

Theoretical calculations using density functional theory, shown in FIGS. 4A-B, demonstrated that the spatial distributions of the highest occupied and lowest unoccupied molecular orbitals of the dipeptides were mainly concentrated on the side-chain indole rings. Specifically, the band gaps (ΔE) of cyclo-FW and cyclo-WW were calculated to be 3.63 eV and 3.56 eV, respectively, indicating their wide-gap semiconductive nature. Without being bound by any particular theory, these data is regarded as implying that the dimerization is mostly driven by 7C-7C interactions between aromatic side-chains, especially the indole rings. Particularly, the aromatic interactions could induce the through-space conjugation of the electron clouds from adjacent indole rings, thus restricting the molecular motions and underlying the molecular basis for quantum confined (QC) regions. The QC effects and restricted molecular activities resulted in the release of excitation energy exclusively as emitted light.

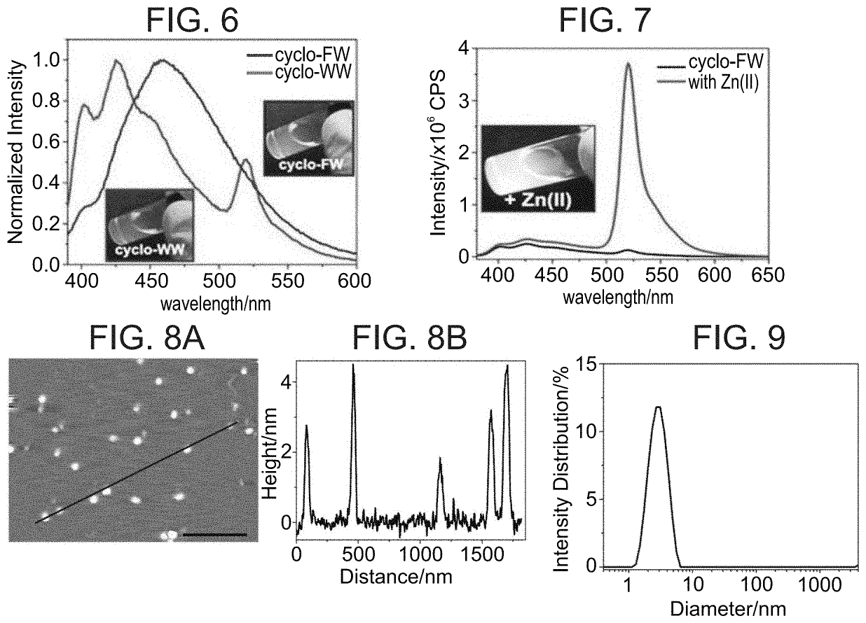

The orderly organized QDs inside the supramolecular self-assemblies result in extensive QC effects along with photoluminescent properties. When excited at 370 nm, the cyclo-dipeptides solutions displayed fluorescence in the visible region, with emission at 460 nm for cyclo-FW and at 425 nm, accompanied by a smaller peak at 520 nm, for cyclo-WW (FIG. 6). Correspondingly, the solutions showed blue-green color under UV light (365 nm) (FIG. 6, insets).

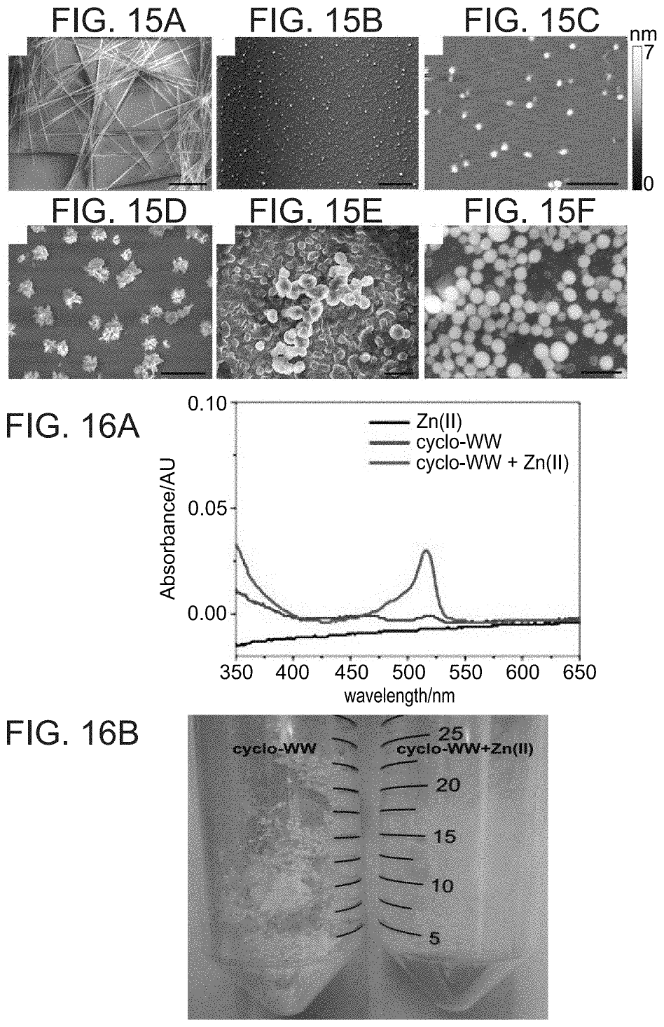

Scanning electron microscopy (SEM) revealed that the two cyclo-dipeptides self-assembled into distinct supramolecular structures, as needle-like crystals were formed by cyclo-FW (FIG. 15A), while cyclo-WW assembled into spherical nanoparticles (FIG. 15B).

The maximal emission of the cyclo-FW assemblies demonstrated a red-shift as excitation wavelength was increased (data not shown), indicating that heterogeneous superstructures of different sizes, arising from the dynamic self-organization, co-existed in the solution.

### Example 2

### Modulation of the Self-Assemblies Morphology and Visible Fluorescence

The doping of the cyclic dipeptide self-assemblies by coordination with metal ions was tested.

Upon introducing Zn(II), the emission of cyclo-WW assemblies (referred to herein as cyclo-WW+Zn(II)) was clearly enhanced, showing a narrow peak at 520 nm with a full width at half maximum of only 18 nm (FIG. 7), leading to a luminous green color under UV light and a quantum yield (QY) of 16% (FIG. 7, inset).

Atomic force microscopy (AFM) experiment indicated the presence of only small nanoparticles, about 3.0 nm in diameter (FIG. 15C; FIGS. 8A-B), approximately the dimension of a dimer. Correspondingly, DLS analysis showed that the size of the structures was about 2.88 nm, with no larger particles present (FIG. 9). The uniform size distribution resulted in a consistent maximal emission, regardless of excitation wavelength (data not shown). These findings demonstrated that following the coordination with Zn(II), the cyclo-WW self-assembly halted at the dimerization stage.

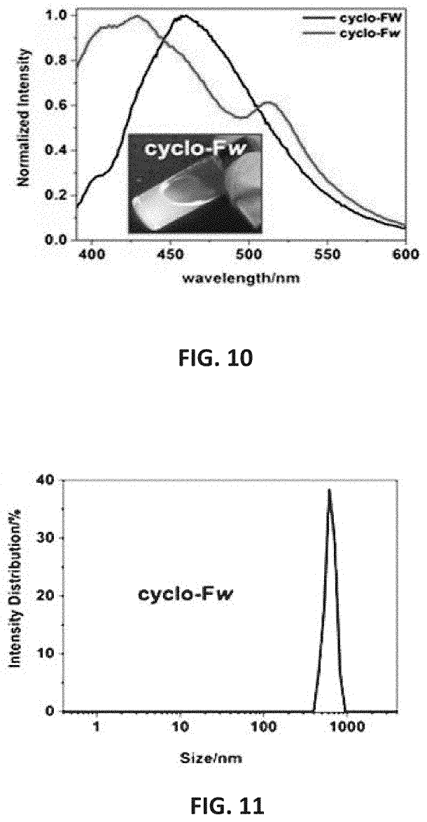

Another strategy to modulate the supramolecular photoluminescence is to substitute the constituents with their enantiomers. W was replaced with its D-type enantiomer in cyclo-FW, hereby designated cyclo-Fw. As shown in FIG. 10, the fluorescent emission shifted to 430 nm. SEM and DLS analyses revealed that cyclo-Fw self-assembled into multi-branched nano-flower architectures (FIG. 15D; FIG. 11), and showed red-shifted maximal emission upon various excitation wavelengths (data not shown).

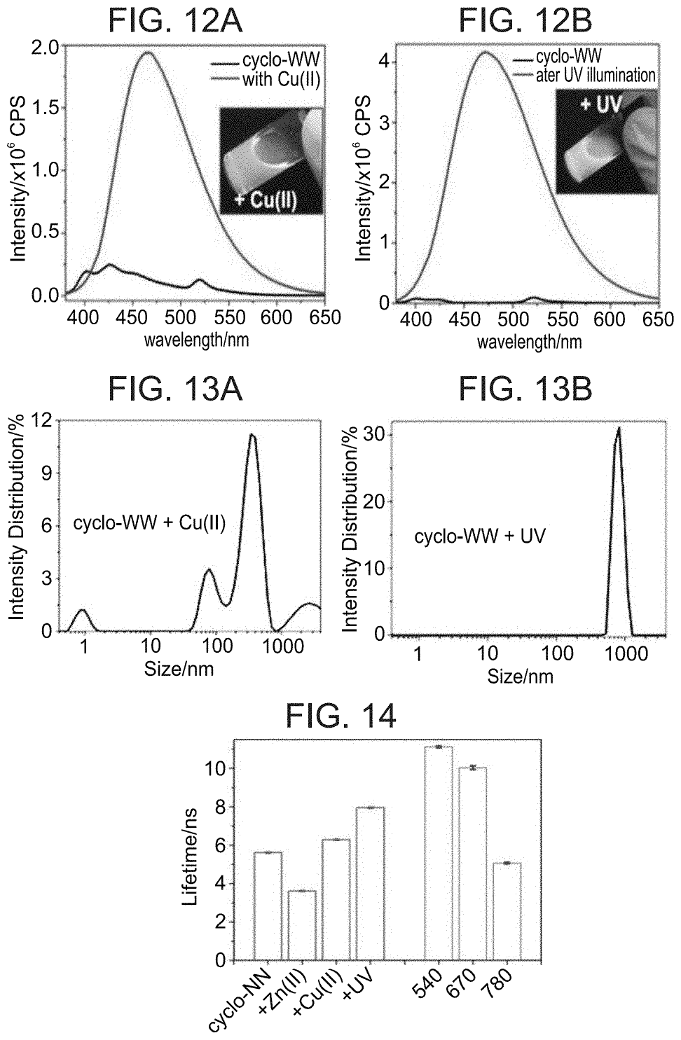

The known reductive property of Trp (W) was further utilized to modulate the fluorescence of the cyclo-dipeptides self-assemblies. As shown in FIG. 12A, when introducing Cu(II), a weak oxidant, into cyclo-WW assemblies (cyclo-WW+Cu(II)), a new fluorescent emission appeared at 465 nm, with a QY of 8%. A similar emission was also observed after irradiation with UV light (365 nm) (cyclo-WW+UV) due to the UV-induced radical oxidation (FIG. 12B), indicating potential use for photo-stimulated applications.

HPLC analysis confirmed that the conversion was complete and the oxidized cyclo-WW was pure (data not shown).

SEM and DLS characterizations showed that the oxidized cyclo-WW self-assembled into spherical nanoparticles, several hundred nanometers to micrometers in diameter (FIGS. 15E and 15F; FIGS. 13A-B). In both cases, the maximal emission red-shifted (data not shown), demonstrating that the spherical nanoparticles further aggregated to form larger particles (not shown). In fact, massive precipitates could be found at the bottom of the cyclo-WW+UV and cyclo-WW+Cu(II) solutions after one week and one month, respectively.

FIG. 14 presents the lifetime statistics of the tested cyclo-WW self-assemblies, as obtained, and upon the modifications described above, extracted from fluorescent decay experiments (not shown). As shown therein, after oxidation, the lifetime increased (6.3 ns for cyclo-WW+Cu(II), 8.0 ns for cyclo-WW+UV) compared to cyclo-WW (5.6 ns), confirming that the redox reactions indeed took place.

The SEM and AFM images of the cyclo-dipeptides QC self-assemblies in MeOH are presented in FIGS. 15A-F. FIG. 15A show needle-like cyclo-FW crystals. FIG. 15B show spherical cyclo-WW nanoparticles. FIG. 15C show dimeric QDs of cyclo-WW+Zn(II). FIG. 15D show Nano-flower architectures of cyclo-Fw. FIGS. 15E and 15F show larger spherical nanoparticles of cyclo-WW+Cu(II) (FIG. 15E) and cyclo-WW+UV (FIG. 15F).

### Example 3

### Mechanistic Insights on the Fluorescence Modulations

The mechanisms underlying the modulation of the fluorescent properties of the self-assemblies was explored.

As shown in FIG. 16A, a new absorption peak at 515 nm, corresponding to ligand (peptide)-to-metal charge transfer, emerged upon mixing cyclo-WW and Zn(II) in solution at a concentration of 5 mM (to thereby promote self-assembly), indicating the formation of coordinated architectures. The new absorption peak resulted in a color change from the original white/light yellow of cyclo-WW to pink (FIG. 16B). In contrast, no new band appeared when Zn(II) was introduced into a monomeric cyclo-WW solution at a concentration of 0.5 mM (in which self-assembly does not occur) (data not shown), confirming that the dimers were indeed the form that complexed with Zn(II). The charge transfer could deliver the excited electrons, resulting in reduced fluorescence decay time, from 5.6 ns of cyclo-WW to 3.6 ns (see, FIG. 14).

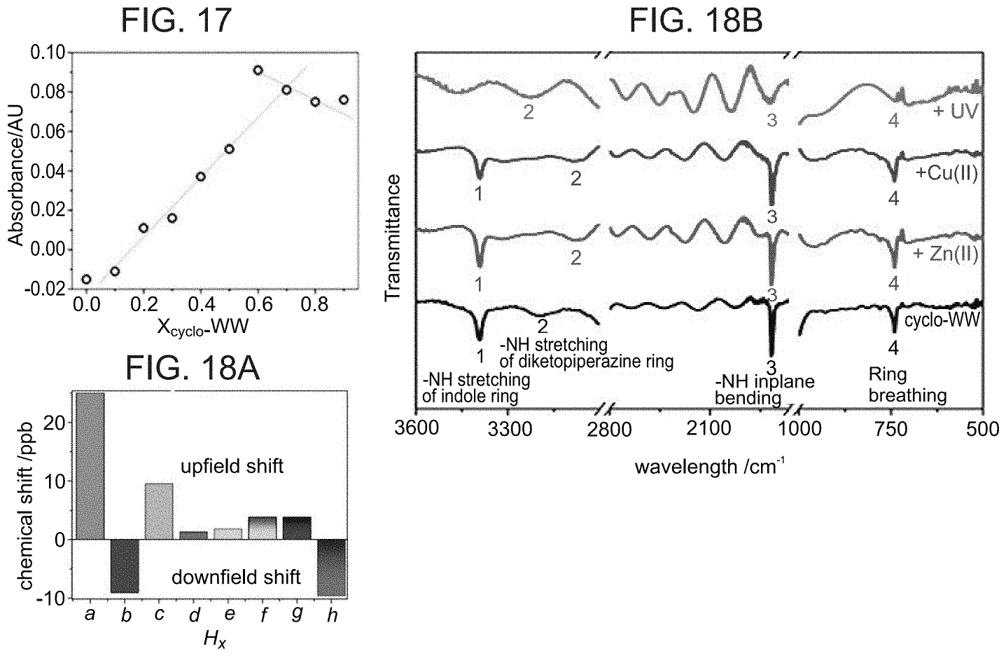

To determine the stoichiometric ratio of cyclo-WW and Zn(II), a Job Plot analysis was performed and is presented in FIG. 17, showing an intersection point at a cyclo-WW proportion of about 0.7. This result indicates that the stoichiometry of cyclo-WW dimers and Zn(II) was approximately 1:1 (0.35:0.3).

The ¹H-NMR spectra shown in FIG. 18A revealed that after coordination, the hydrogen atoms in the backbone showed downfield shifting, indicating that the shielding effect became weaker. This suggests that the diketopiperazine ring contributed to the coordination by supplying electrons to interact with Zn(II). In contrast, the hydrogen atoms in the indole rings showed upfield shifting, indicating that the delocalization of the π-electrons on the aromatic rings became stronger. This demonstrates that the indole rings formed aromatic interactions with each other and through-space conjugation of electrons took place, consistent with the theoretical calculations. Fourier-transform infrared spectroscopy (FTIR) characterizations, shown in FIG. 18B, demonstrated that in the presence of Zn(II), the N—H stretching vibration in diketopiperazine rings red-shifted from the original 3200 cm⁻¹ to 3071 cm⁻¹ due to the decrease of the bond energies resulting from the metal ion adsorption (FIG. 18B, peak 1), indicating that the nitrogen atoms in the backbone diketopiperazine rings contributed to the complexation with Zn(II).

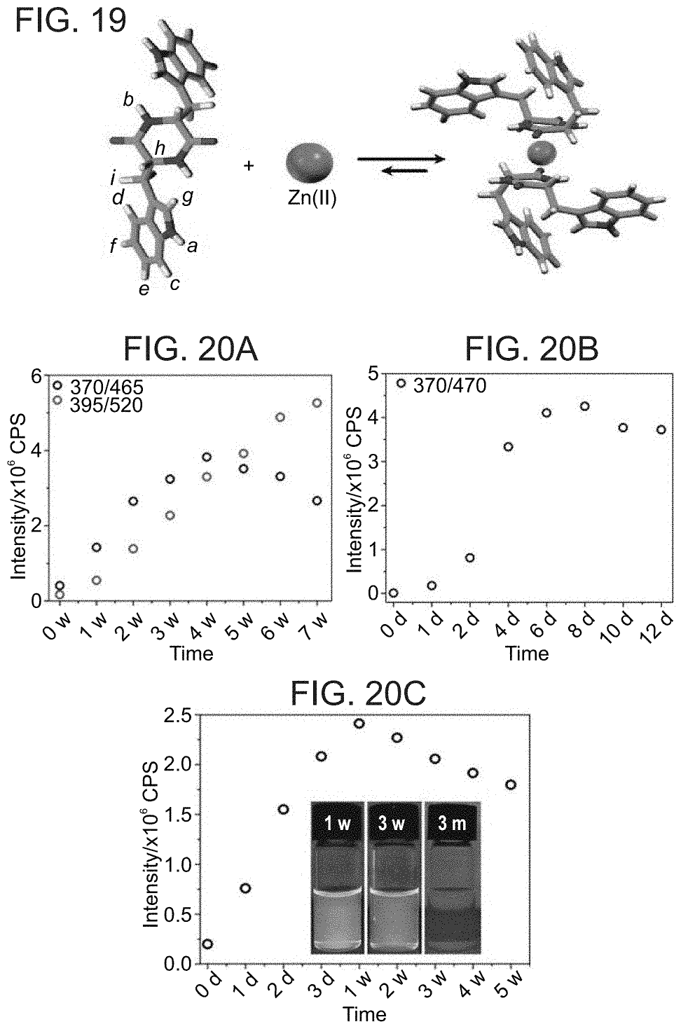

A plausible molecular mechanism of cyclo-WW coordination with Zn(II) is depicted in FIG. 19, based, inter alia, on the NMR and FTIR data. The Zn(II) ion is embedded in a dimer of cyclo-WW, coordinating with two diketopiperazine rings, while the side-chain indole rings form π-π interactions. Without being bound by any particular theory, it is suggested that the complexation induces hindrance against further aggregation of the dimers, thus resulting in the dimers separated from each other and showing stable photoluminescence regardless of the excitation wavelength.

The larger size and aggregating nature of the cyclo-WW+Cu(II) and cyclo-WW+UV nanospheres resulted in time-resolved parabolic evolution of the emission intensities, as shown in FIGS. 20A-B. In fact, the weak oxidation of Cu(II) induced slower aggregation dynamics, thus leading to a continuous increase of the fluorescent emission at 465 nm, with an excitation of 370 nm, persisting over one month (FIG. 20A, black circles). This also resulted in larger aggregations with a long-wavelength 520 nm emission, following excitation at 395 nm, constantly increasing over this period (FIG. 20A, red circles). As a control, the emission of cyclo-WW+Zn(II) decayed due to quenching (FIG. 20C).

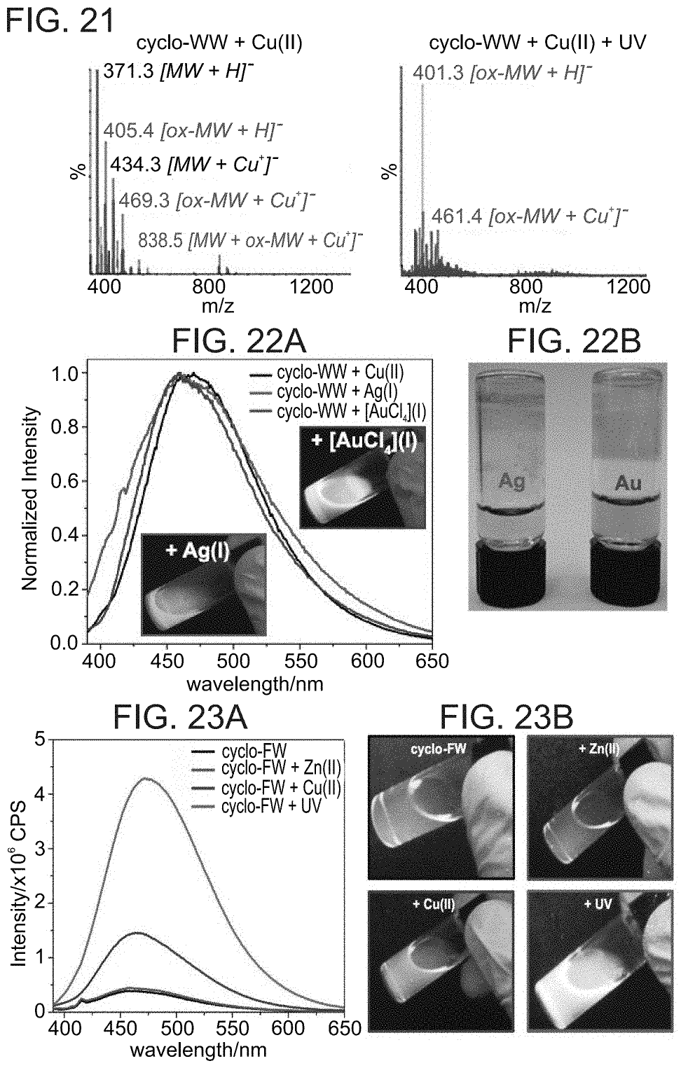

The redox reaction was verified by MS analysis, showing an m/z corresponding to the MW of oxidized cyclo-WW in the cyclo-WW+Cu(II) solution, in addition to the dominant peak of native cyclo-WW (FIG. 21, left panel).

When combining both Cu(II) oxidation and UV irradiation (cyclo-WW+Cu(II)+UV), the synergistic effect dramatically accelerated the oxidation reaction, leading to an MS profile predominantly comprised of the m/z of oxidized cyclo-WW (FIG. 21, right panel). Additionally, in both mass-spectra, the MW of Cu(I)-conjugated peptides was detected, indicating that the Cu(II) ions have been reduced. In contrast, in cyclo-WW+Zn(II), a chloride ion was always integrated, thus counteracting the positive charge of Zn(II) (data not shown).

The oxidation mechanism of Cu(II) was further verified using other metal ions with higher oxidative capability. As shown in FIG. 22A, when replacing Cu(II) with Ag(I) or [AuCl₄](—I), the sample solutions showed the same emission spectra with a maximum at 465 nm and blue-green color under UV light. The intense redox reduced the metal ions to elementary metals (FIG. 22B).

The different modulation mechanisms of Zn(II) and Cu(II) could also be confirmed by mixing with cyclo-FW, which did not complex with Zn(II) but showed enhanced fluorescence at 465 nm with a QY of 12% in the presence of Cu(II) (FIGS. 23A-B). The results show similar fluorescent emission in the absence or presence of Zn(II), indicating that cyclo-FW did not coordinate with Zn(II). In contrast, in the presence of Cu(II) and UV irradiation, intense fluorescence was detected, demonstrating the different mechanisms of modulating the supramolecular fluorescence by Zn(II) vs. Cu(II)/UV.

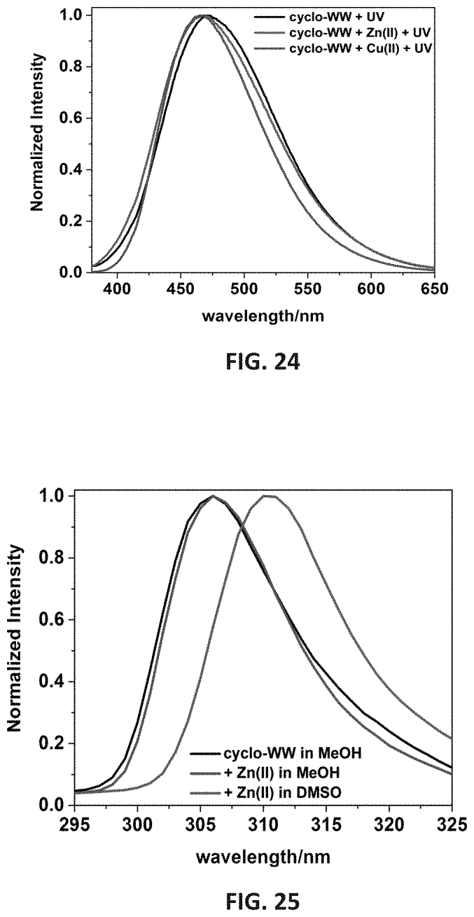

The similar emission spectra of cyclo-WW+Cu(II) and cyclo-WW+UV suggested that the oxidized cyclo-WW did not complex with metal ions. As shown in FIG. 24, cyclo-WW+Zn(II)+UV had the same fluorescence emission as cyclo-WW+UV and cyclo-WW+Cu(II)+UV, rather than that of cyclo-WW+Zn(II) (FIG. 7).

The FTIR analysis demonstrated that in cyclo-WW+Cu(II) and cyclo-WW+UV, the vibration of N—H stretching of indole rings (3393 cm⁻¹) significantly attenuated (FIG. 18B, peak 1), indicating that the redox took place at the N—H bonds of the side-chains. Combined with the MS data, it is assumed that the two nitrogen atoms of the indole rings were conjugated with oxygen to form —N═O bonds. In addition, the intensity of the ring breathing vibration (742 cm⁻¹) also declined (FIG. 18B, peak 4), suggesting that the redox severely disrupted the conformation of the rings. Without being bound by any particular theory, it is assumed that the oxidation changed the electronic density and steric structures of the indole rings, thereby hindering the interactions of oxidized cyclo-WW with metal ions.

### Example 4

### Near-Infrared (NIR) Fluorescence

One of the characteristics of QC materials is the dependence of their emission colors on particles size. The excitation-dependent photoluminescent feature of the self-assemblies as exemplified herein have prompt the present inventors to introduce inhomogeneous size distributions to the self-assembles, for instance by using solvents that can facilitate the self-assembly, so that their emissions be red-shifted to even longer wavelengths.

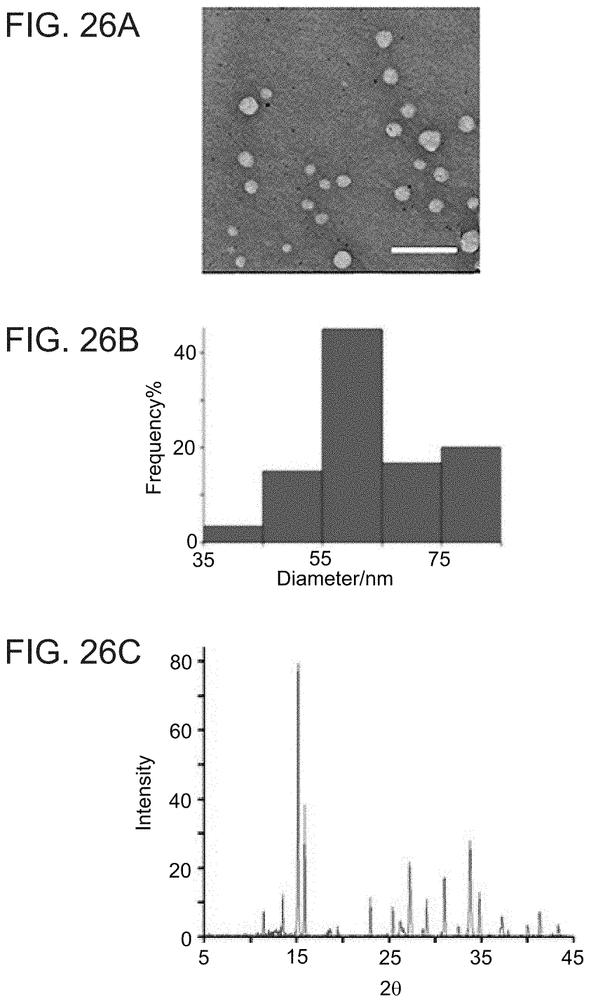

MeOH was replaced by the more polar DMSO as a solvent for cyclo-WW+Zn(II), and the extensive aggregation resulted in molecular excitation red-shift, from 305 nm in MeOH to 310 nm (FIG. 25), and the dimers self-assembled into larger spherical nanoparticles, 63.6±12.2 nm in diameter (FIGS. 26A-B).

X-ray diffraction analysis (FIG. 26C) revealed distinct sharp peaks with high intensities, indicating the high crystallinity of the nanospheres, consistent with the crystallized nature of the QDs.

These results illustrated the well-organized, periodical nano-lattice arrangement within the assemblies, thus confirming the directional organization of the dimeric QDs and the extensive internal QC effects.

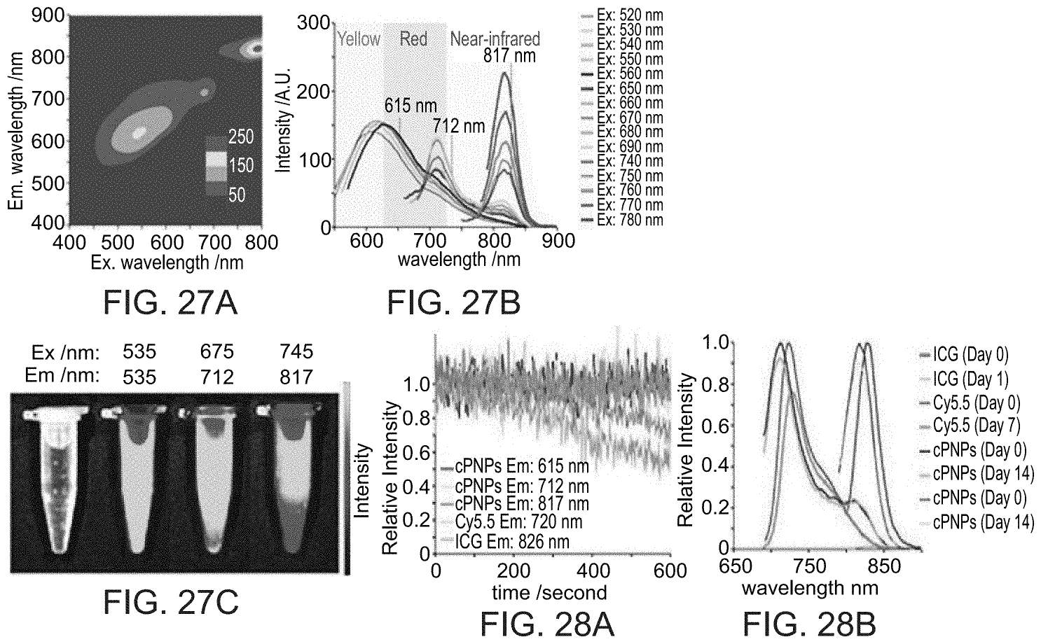

As shown in FIGS. 27A-C, fluorescent characterization of the cyclo-WW+Zn(II) nanospheres in DMSO demonstrated both visible and NIR fluorescence under a wide range of excitation wavelengths (FIG. 27A).

Specifically, the peaks at 615 nm, 712 nm and 817 nm were emitted using excitation wavelengths between 520 and 780 nm (FIG. 27B), with lifetimes of 11.1 ns, 10.0 ns and 5.1 ns, respectively (see, FIG. 14), suggesting that as aggregation progressed, the excited electrons became more stable. This indicates that the supramolecular structures can be used as bio-inspired alternatives for stable, long-term imaging.

Correspondingly, the solution displayed distinct colors under different excitation wavelengths (FIG. 27C). The QYs were measured to be 18% and 27% for emissions at 712 nm and 817 nm, respectively.

Photobleaching evaluation experiments demonstrated that the photoluminescence of the nanospheres formed of cyclo-WW+Zn(II) in DMSO remained stable after continuous irradiation for 600 seconds, compared to the significant fluorescence decay observed for organic fluorescent dyes (indocyanine green (ICG) and cyanine 5.5 (Cy5.5) (FIG. 28A). In addition, time-resolved characterization showed the fluorescence of the nanospheres to be stable even after exposure to natural light for two weeks, indicating their high photostability. In contrast, ICG lost all fluorescence after 1 day and Cy5.5 lost 20% of the fluorescence after 1 week (FIG. 28B).

### Example 5

### QC Fluorescent Self-Assemblies as Photostimulated Device and as an Imaging Probe

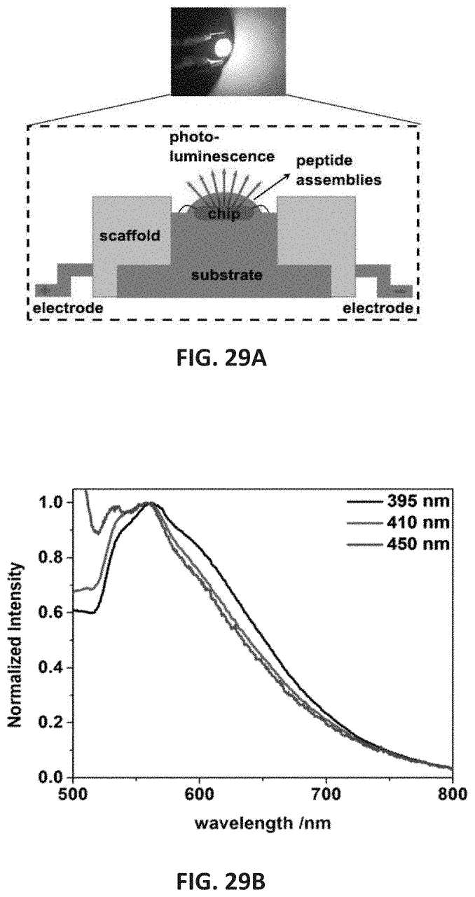

The photoluminescent nature endows the peptide QC self-assemblies the ability to be used for photo-stimulated devices, such as light emitting diodes (LEDs). By applying a mixture of dried (upon solvent evaporation) cyclo-WW+Zn(II) dots (formed in MeOH) and polydimethylsiloxane (PDMS) onto an indium gallium nitride (InGaN) chip, an exemplary LED device using peptide self-assemblies as phosphors was fabricated, as schematically illustrated in FIG. 29A. When applying voltages, bright green light was illuminated, as shown in the inset of FIG. 29A.

Spectroscopic investigations demonstrated an emission around 550 nm regardless of the excitation wavelength (FIG. 29B), thus showing remarkable emission specificity. The red-shift of 30 nm from 520 nm (also shown in FIG. 7) is assumed to be attributed to aggregation that occurred during MeOH evaporation.

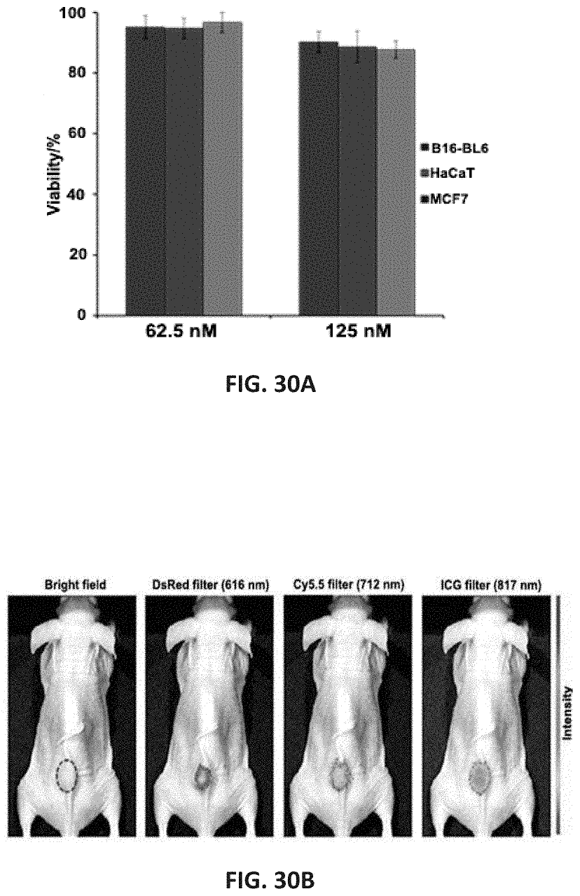

The bioinspired nature and the notable emission up to the NIR region indicate a potential utilization of cyclo-WW+Zn(II) nanospheres in biological systems. In in vitro cytotoxicity analysis, the peptide nanoparticles showed good biocompatibility towards B16BL6 (murine melanoma cell line), MCF-7 (human breast cancer cell line) and HaCaT (human skin cell line) cells (FIG. 30A).

Subcutaneous injection of the nanospheres into nude mice followed by NIR fluorescence imaging revealed distinct visible and NIR fluorescent signals at the injection site (FIG. 30B). The fluorescent signals were stable, showing no decay for one week, thus highlighting the possibility of utilizing the photoluminescent QC assemblies for in vivo bio-imaging applications. The advantage of easy modifications, such as specific targeting and controllable assembly/dis-assembly, facilitates simple functionalization of the assemblies, thus exemplifying their use as targeted therapy and controllable drug release.

### Example 6

### Self-Assemblies Using Additional Aromatic Dipeptides

The roles that aromatic side-chains play during self-assembly have prompt the present inventors to test the effect of the side chain aromatic moieties on the supramolecular morphologies and photoluminescent properties.

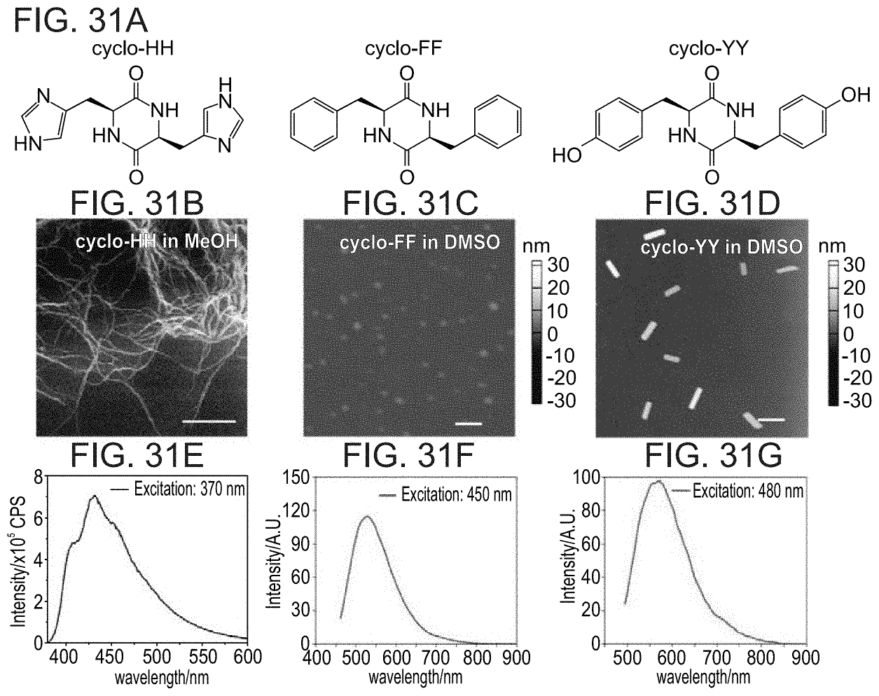

In preliminary studies, three cyclo-dipeptides comprised of different aromatic amino acids, cyclo-dihistidine (cyclo-HH), cyclo-diphenylalanine (cyclo-FF), and cyclo-dityrosine (cyclo-YY), shown in FIG. 31A, were examined under the same conditions.

Morphological characterization showed that the cyclo-dipeptides self-assembled into diverse supramolecular structures. Specifically, cyclo-HH formed nanofibers in MeOH (FIG. 31B), cyclo-FF formed spherical nanoparticles in DMSO (FIG. 31C), and cyclo-YY assembled into nanorods in DMSO (FIG. 31D).

AFM analysis demonstrated small aggregates less than 4 nm in height at a lower concentration (0.5 mM), with dots for cyclo-HH and cyclo-FF and thin nanofibers for cyclo-YY (FIGS. 32A-C). This suggested that like cyclo-FW and cyclo-WW, the self-assemblies of these three cyclo-dipeptides were also composed of QC intermediates.

Fluorescent characterization demonstrated that the distinct morphologies presented different photoluminescence, with cyclo-HH nanofibers (formed when a cyclo-dipeptide concentration of 5 mM is used) showing emission at 430 nm (Ex: 370 nm) (FIG. 31E), cyclo-FF nanoparticles at 530 nm (Ex: 450 nm) (FIG. 31F) and cyclo-YY nanorods at 570 nm (Ex: 480 nm) (FIG. 31G).

### Example 7

The present inventors have further studied all aromatic cyclo-dipeptides, their self-assembly and relevant photoluminescent properties, modulated the supramolecular structures by doping with zinc ions, upon a unique environment switching mechanism, and accordingly, the photoluminescent behaviors. As can be seen, bioinspired nanostructures were demonstrated to be usable as biocompatible phosphors for engineering LEDs, and thus as bio-organic photoactive alternatives for eco-friendly electronics.

Self-Assembly and Intrinsic Photoluminescence:

As shown hereinabove, hydrogen bonding and aromatic interactions are the main driving forces during aromatic short peptides self-assembly, leading to the optoelectronic properties of the peptides self-assemblies. Both can decrease the energy bandgap and facilitate aggregation-induced photoluminescence in the visible light region.

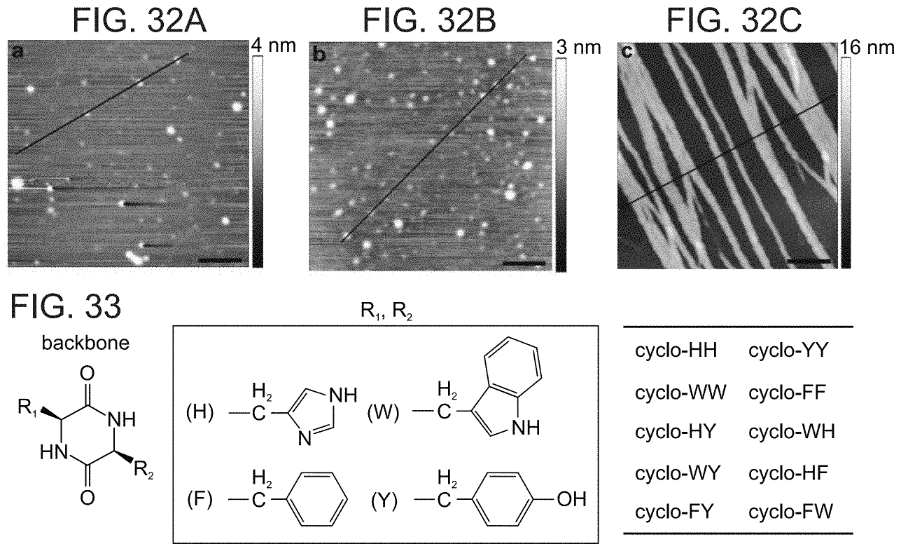

The self-assembly and corresponding photoluminescent properties of a total of 10 cyclo-dipeptides made of various combinations of all natural aromatic amino acids, including histidine (H), W, F and tyrosine (Y) were studied, as shown in FIG. 33.

Briefly, after dissolving the cyclo-dipeptide powder in methanol and heating to 80° C., most peptides were dissolved (except cyclo-FF and cyclo-YY).

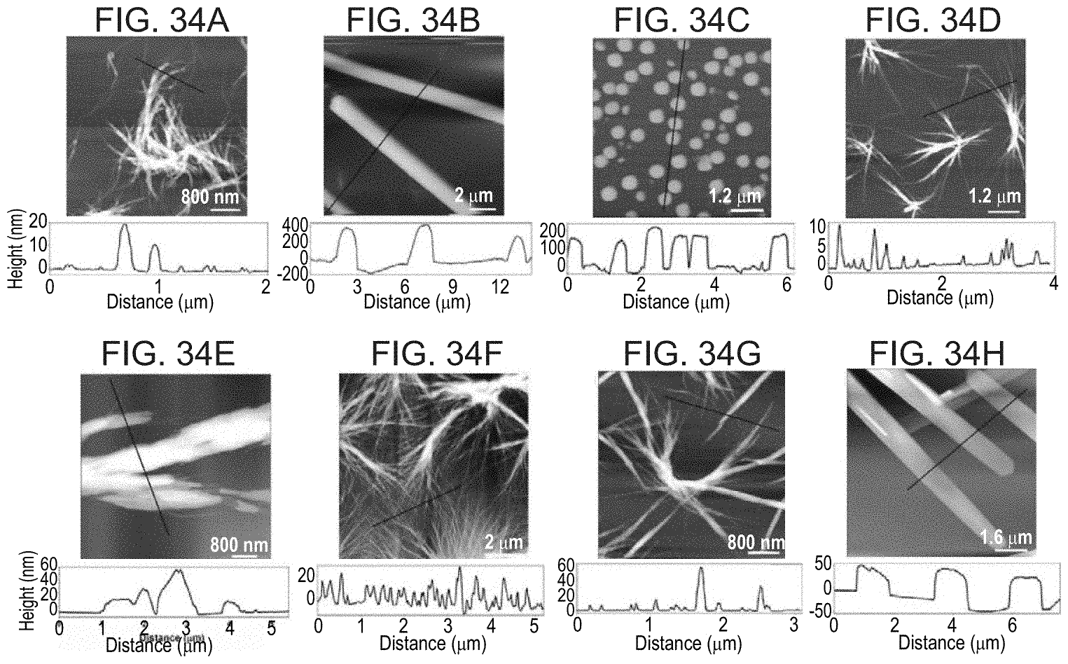

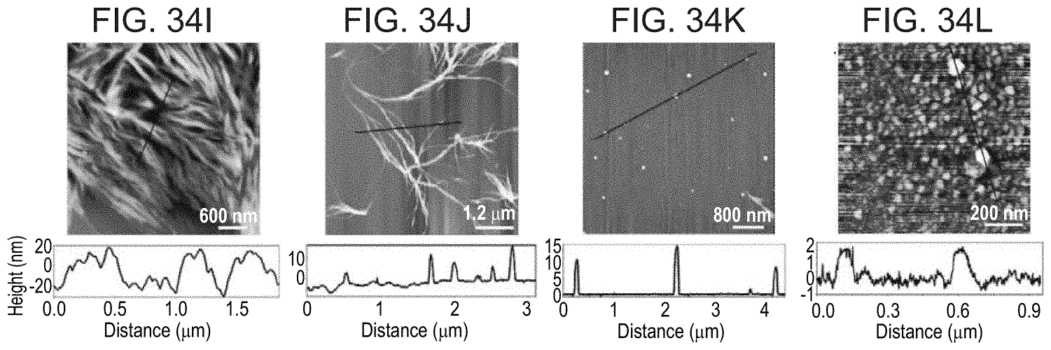

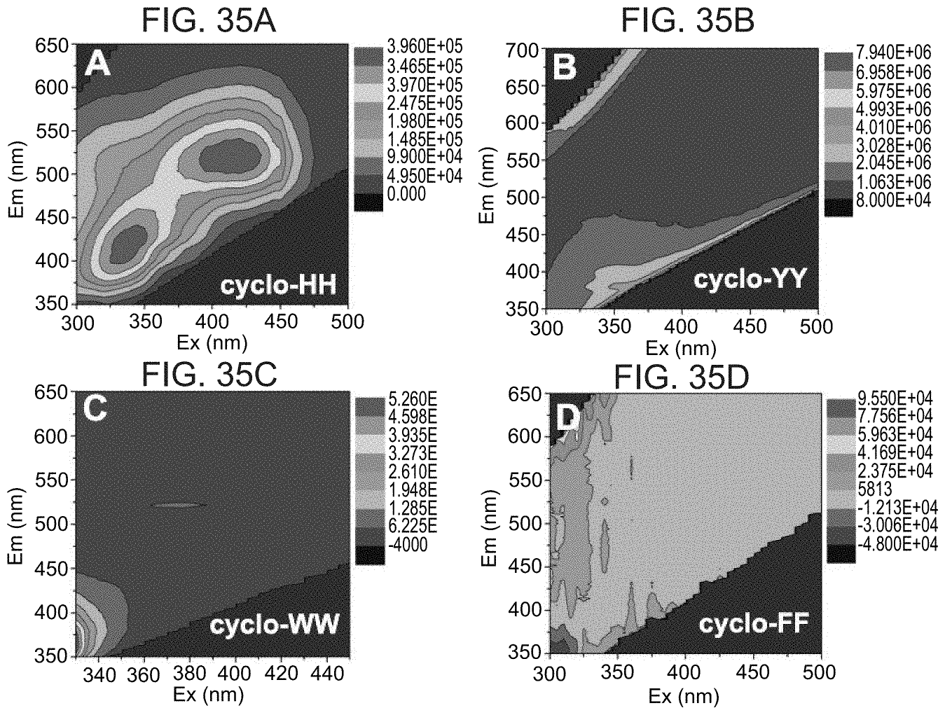

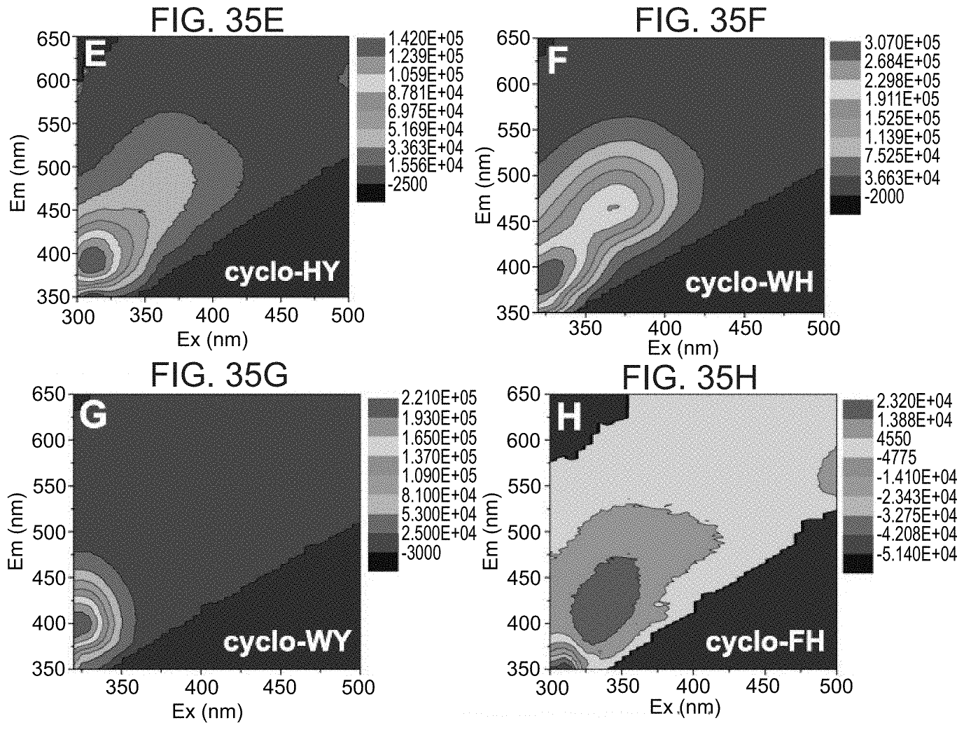

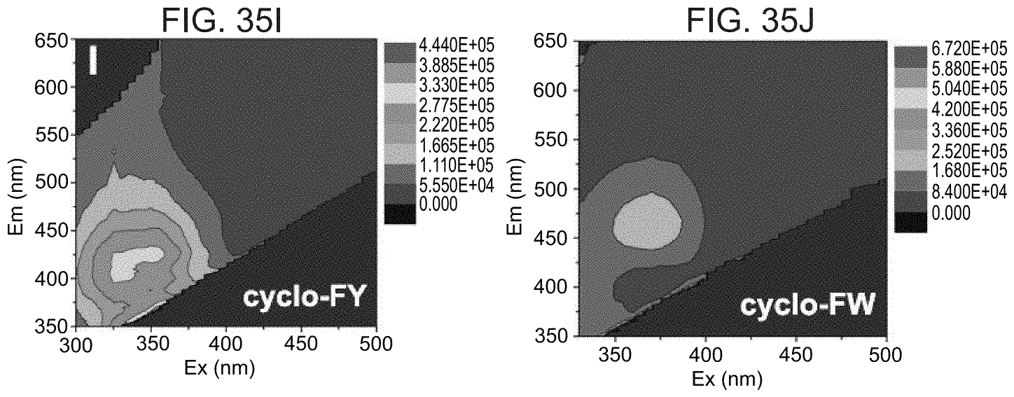

Atomic force microscopy (AFM) analysis, shown in FIGS. 34A-L and Table 1 below, demonstrated that the cyclo-dipeptides self-assembled into diverse architectures with dimensions ranging from dozens to hundreds of nanometers, including nanospheres (cyclo-WW), nanofibers (cyclo-HH, cyclo-FF, cyclo-HY, cyclo-WH, cyclo-WY, cyclo-HF, cyclo-FY) and platelets (cyclo-YY, cyclo-FW). This implies that the supramolecular morphologies can be finely modulated by simply modifying the peptide residues. High-magnification AFM revealed that alongside the larger supramolecular morphologies, nanoparticles of 2 to 10 nm were also present. For example, along with the nanofibers, discrete, dot-like nanoparticles could be detected in the cyclo-HY and cyclo-WH systems (see, FIGS. 35K and 35J), implying the hierarchical assembly in these bio-organic architectures. These findings support the notion that the aromatic cyclo-dipeptides first oligomerize into nanodots, which comprise the intermediates to further assemble into larger nano s tructures.

Aromatic amino acids show intrinsic fluorescence with emission at 360 nm (Ex: 220 nm), 348 nm (Ex: 280 nm), 282 nm (Ex: 257 nm) and 274 nm (Ex: 220 nm) for H, W, F, and Y, respectively [e.g., M. O. Iwunze, J. Photochem. Photobiol., A 2007, 186, 283]. These wavelengths are all in the UV light region (<400 nm), thus severely limiting their application in biological systems and for ecofriendly photoelectronics.

Density functional theory (DFT) calculations demonstrated that although the spatial distributions of the highest occupied molecular orbitals were dispersed on both the backbone diketopiperazine and the side-chain aromatic rings, the electron clouds distribution was only concentrated on the side-chain aromatic rings for the lowest unoccupied molecular orbitals of the cyclodipeptides (data not shown), indicating that the aggregations, along with the potential photoactive properties, were mostly driven by π-π interactions between aromatic side chains.

As shown in Table 1, the band gaps (ΔE) were calculated to be between 3.56 and 4.55 eV, indicating the widegap semiconducting nature of the dipeptide assemblies.

The aromatic interactions could decrease the electron transition energy and lead to the through-space conjugation of the electron clouds, thus inducing the red shift of the assemblies.

TABLE 1

| | | | Excitation (λ_{Ex}) | Emission (λ_{Em}) | QY^{a} | Lifetime (τ) |
|---|---|---|---|---|---|---|
| | ΔE [eV] | Structures | (nm) | (nm) | (%) | (ns) |
| cyclo-HH | 3.61 | nanofibers | 340 | 425 | 3 | 4.89 ± 0.02 |
| | | | 420 | 520 | 7 | 6.48 ± 0.04 |
| cyclo-YY | 4.00 | platelets | — | — | — | — |
| cyclo-WW¹ | 3.56 | nanospheres | 370 | 440 (strong) | 2 | 5.45 ± 0.03 |
| | | | | 520 (weak) | | |
| cyclo-FF | 4.50 | nanofibers | — | — | — | — |
| cyclo-HY | 4.00 | nanofibers | 310 | 390 | 3 | 0.003 ± 0.034 |
| cyclo-WH | 3.56 | nanofibers | 320 | 380 (strong) | 3 | 0.07 ± 0.01 |
| | | | 370 | 465 (weak) | 2 | 11.37 ± 0.05 |
| cyclo-WY | 4.55 | nanofibers | 320 | 400 | 3 | 0.232 ± 0.004 |
| cyclo-HF | 4.19 | nanofibers | 340 | 410 | 2 | 3.29 ± 0.03 |
| cyclo-FY | 3.94 | nanofibers | 350 | 430 | 3 | 0.022 ± 0.003 |
| cyclo-FW¹ | 3.63 | platelets | 370 | 465 | 2 | 4.52 ± 0.05 |
| cyclo-HH + | — | nanodots | 395 | 490 | 15 | 4.63 ± 0.01 |
| Zn(II) | | | | | | |
| cyclo-WW + | — | nanodots | 370 | 520 | 12 | 3.63 ± 0.01 |
| Zn(II)¹ | | | | | | |
| cyclo-HY + | — | nanodots | 380 | 490 | 10 | 4.79 ± 0.01 |
| Zn(II) | | | | | | |

^{a}During QY measurements, due to the limitation of instrument parameters, the excitation was set at 350 nm if the maximal excitation wavelength of samples was less than 350 nm.

Table 1 further presents the maximal excitation (λEx) and emission (λEm) wavelengths of the cyclo-dipeptide self-assemblies, and FIGS. 35A-J present the corresponding excitation-resolved emission contour profiles. Except cyclo-YY, cyclo-FF (with no detectable emissions) and cyclo-HY (λEm at 390 nm), all other cyclo-dipeptide self-assemblies showed fluorescence of no less than 400 nm. Cyclo-HH, cyclo-WW and cyclo-WH self-assemblies showed two λEm each, namely 425 nm (Ex: 340 nm) & 520 nm (Ex: 420 nm), 440 nm & 520 nm (Ex: 370 nm), and 380 nm (Ex: 320 nm) & 465 nm (Ex: 370 nm), respectively. These spectroscopic findings indicated the hierarchical organization and the size-encoded photoluminescence of the assemblies, consistent with the AFM results.

In addition, as further shown in Table 1, the photoluminescence of the self-assemblies showed remarkable stability, with fluorescence lifetimes (τ) of nearly 5 nanoseconds and cyclo-WH showing the longest τ of up to 11 nanoseconds for the 465 nm emission.

Zinc Ions Mediated Photoluminescence Enhancement:

As shown in Table 1, the dominant emissions of the peptide self-assemblies were between 400 and 465 nm, in the blue and blue-green region, with quantum yield (QY) of 2-3% for most self-assemblies, and of 7% for the 520 nm emission for cyclo-HH.

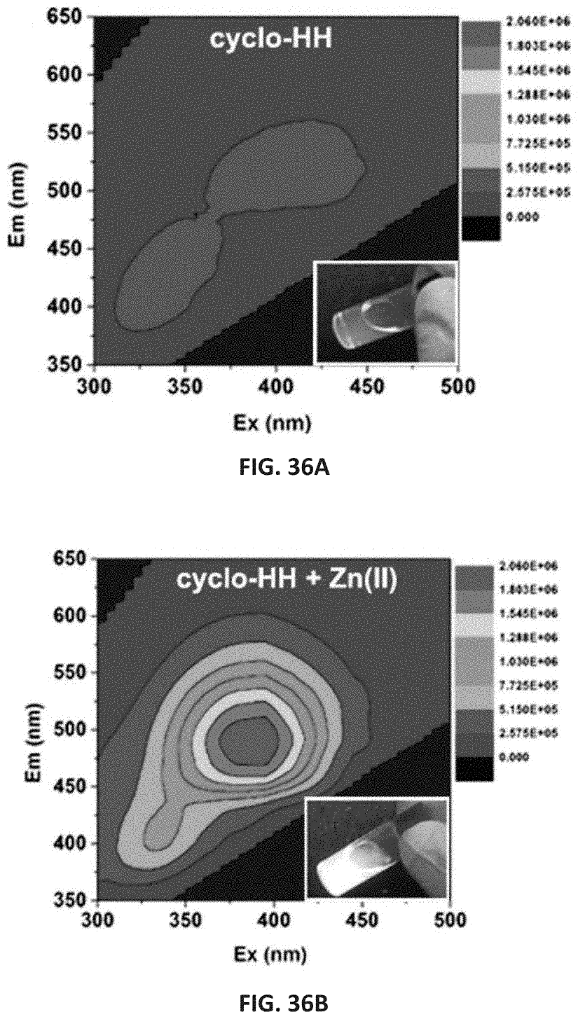

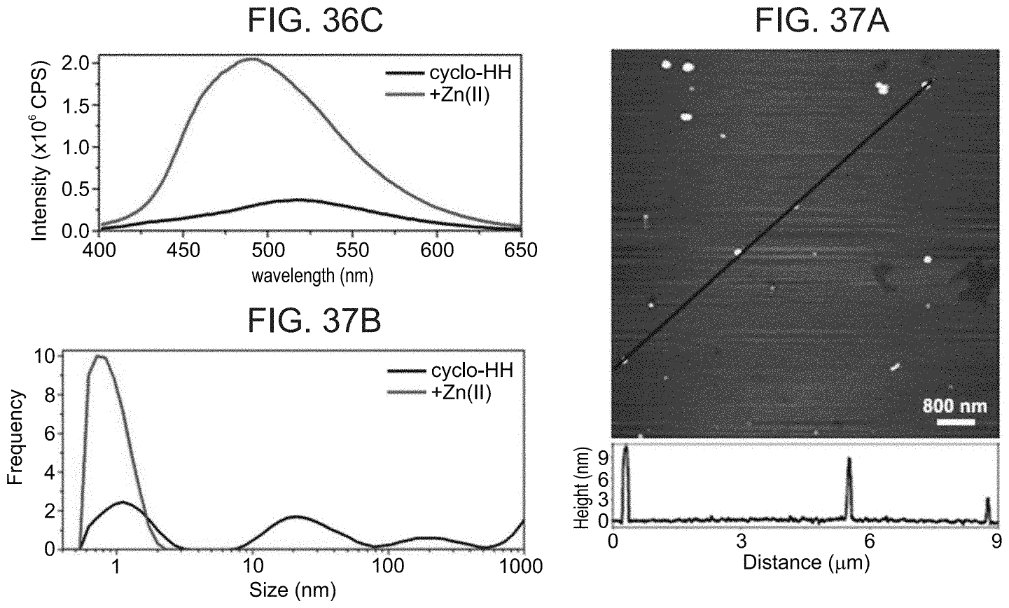

Aiming at increasing the efficiency, Zn(II) was introduced to the aromatic cyclodipeptide solutions. As shown in Table 1 and FIGS. 36A-C, fluorescence characterization demonstrated that the emission intensity of the cyclo-HH solution was significantly enhanced in the presence of Zn(II), with the λEm detected at 490 nm (Ex: 395 nm) and τ of 4.63 nanoseconds. As further shown in Table 1, the photoluminescence efficiency was improved, with an enhanced QY of 15% for cyclo-HH+Zn(II). The emission of the cyclo-HY solution was also enhanced to some extent in the presence of Zn(II), with the λEm detected at 490 nm (Ex: 380 nm), τ of 4.79 ns and a QY of 9%, similarly to cyclo-WW. The other dipeptides show less noticeable changes in the presence of Zn(II) (data not shown).

The coordination mechanism between zinc ions and cyclo-HH was studied further.

As shown in FIGS. 37A-B, AFM characterizations demonstrated that only dot-like nanoparticles, several nanometers in height, were present in the cyclo-HH+Zn(II) solution (FIG. 37A). Dynamic light scattering (DLS) experiments revealed that the dominant particles in the solutions were only several nanometers size, compared to dozens to hundreds of nanometers for the same peptides in the absence of zinc ions (FIG. 37B). These results indicated that the coordination with Zn(II) inhibited the hierarchical organization of cyclo-HH, resulting in separation of the oligomers from each other, thereby leading to their stabilization.

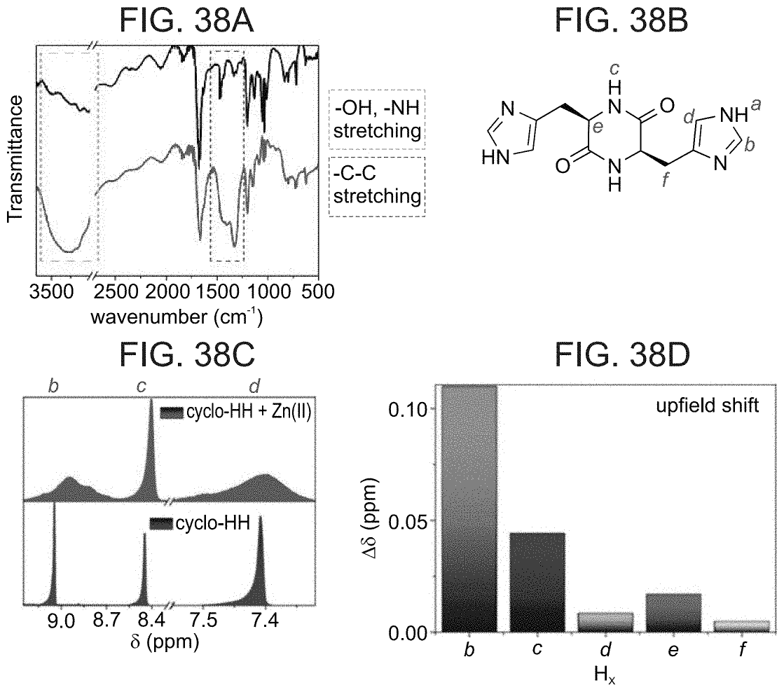

The Fourier-transform infrared spectroscopy (FTIR) analysis shown in FIG. 38A indicates that after Zn(II) coordination, the —NH stretching vibration peaks of the cyclo-HH became narrower and sharper, indicating that less hydrogen bonds were formed and many —NH moieties remained free. In addition, the insert backbone C—C stretching vibration peaks became relatively stronger, indirectly confirming that the active nitrogen atoms extensively participated in complexation with Zn(II) and the coordination distinctly hindered the IR absorption of the relevant chemical bonds. This confirms that the active nitrogen atoms extensively participated in complexation with Zn(II), as demonstrated herein for cyclo-WW+Zn(II).

¹H NMR analysis, shown in FIGS. 38B-D, indicated that the chemical shifts (Δδ) of cyclo-HH hydrogen atoms exhibit band broadening along with upfield shifting following coordination with Zn(II) (FIGS. 38B-C), especially for the imine protons of the side-chain imidazole ring (marked with b, Δδ=0.11 ppm) (FIG. 38D), indicating that the shielding effect of the electron clouds became stronger. The results suggest that the imidazole ring, rather than the diketopiperazine rings as in the cyclo-WW+Zn(II) case, coordinated with Zn(II) through the imine nitrogen atom, thus decreasing the attraction of electrons outside the proton at the b position.

Molecular Dynamics (MD) Simulations and Free Energy Calculations:

Molecular dynamics (MD) simulations were further utilized to study the self-assembly of cyclo-HH.

MD simulations and free energy calculations were performed to investigate the self-assembly of two systems: (1) cyclo-HH dipeptides in methanol and (2) cyclo-HH+Zn(II) in methanol. Five MD simulation runs were performed for each system, in CHARMMfmer using the CHARMM36 force field, with a Drude polarizable force field to account for Zn(II) mediated interactions. In both systems, multiple (64) peptides were placed in a periodic boundary conditions box in the absence or presence of Zn(II). Details of the MD simulations are provided in the Supplementary Information.

Characterization of interactions between pairs of cyclo-HH dipeptides, and between cyclo-HH dipeptides and Zn(II): In the MD simulations the cyclo-HH dipeptides self-assembled into clusters ranging from two and up to seventeen peptides. The analysis focused on the cyclo-HH dipeptide: cyclo-HH dipeptide interactions and Zn(II): cyclo-HH dipeptide interactions within the clusters. Distance criteria were defined to characterize the interactions between cyclo-HH dipeptides (hydrogen bonds between histidine side chain atoms, diketopiperazine backbone atoms, as well as histidine side chain atoms and diketopiperazine backbone atoms). The criteria were used to identify the ordered structures (containing β-bridge like interactions between a pair of two cyclo-HH or between three or four adjacent dipeptides involved in elongated β-bridge like interactions) and amorphous structures (without any particular β-bridge like interactions between cyclo-HH dipeptides) within the clusters. Additional distance criteria were defined characterizing the interactions between cyclo-HH and Zn(II) (coordination between Zn(II) and histidine sidechain atoms or diketopiperazine backbone atoms) to identify how Zn(II) coordinate with the ordered or amorphous arrangements of cyclo-HH within the clusters.

Energetic analysis of clusters formed by cyclo-HH in the absence and presence of Zn(II): Interaction and association free energy calculations were performed using the MM-GBSA approximation to investigate the mechanism and driving forces leading to the association and stabilization of cyclo-HH dipeptides and Zn(II) within the clusters, in the first moments of self-assembly prior to the formation of highly-ordered structures. Each calculation was performed for each individual cluster, and the resulting energy values were normalized by the number of cyclo-HH dipeptides within the cluster and decomposed into polar and non-polar components.

Across all simulations, visual inspection confirmed that highly disordered clusters of cyclo-dipeptides deformed and reformed, ensuring that the cyclo-dipeptides were not trapped in a local energetic minimum in a given simulation, while facilitating the formation of structures with higher order or symmetry (data not shown).

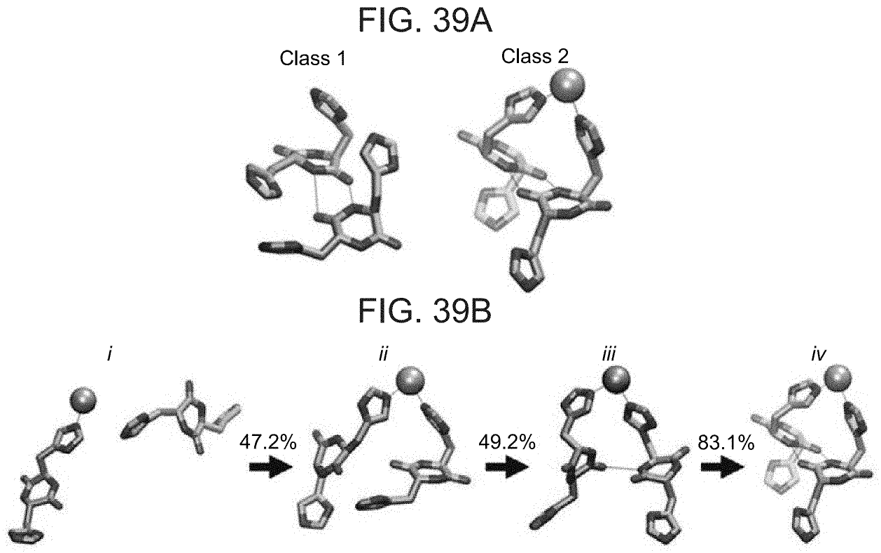

Focus was put on the formation of the elementary interactions between a pair of cyclo-dipeptide molecules resulting in an ordered structure. In the cyclo-HH methanol solution, cyclo-HH dipeptides tended to form ordered conformations where both imidazole side chains were located on the same side of the backbone diketopiperazine rings, namely “Class 1” β-bridge like conformation (see, FIG. 39A, left panel). In contrast, in the cyclo-HH+Zn(II) system, pairs of cyclo-HH dipeptides tended to form ordered conformations such that the imidazole side chains of the two interacting dipeptides were on the opposite sides of the backbone, namely “Class 2” β-bridge like conformation (See, FIG. 39A, right panel), as also confirmed by subsequent crystallographic characterization following the hierarchically oriented organization principle. The “Class 2” β-bridge like conformations were primarily observed only if Zn(II) was coordinated between two H imidazole rings belonging to opposite, β-bridge bonded cyclo-HH dipeptides (FIG. 39A). Otherwise, if the pair of cyclo-HH dipeptides was not coordinated with Zn(II), the molecules tended to form “Class 1” β-bridge like conformations. These findings indicate that the introduction of Zn(II) indeed affected the association of cyclo-HH dipeptides and that the “Class 2” β-bridge like conformation was stabilized by Zn(II) through “locking” the pair of cyclo-HH molecules.

In the cyclo-HY+Zn(II) system, which showed weaker photoluminescence enhancement compared to cyclo-HH+Zn(II), the Zn(II) ions did not steer any conformational alternations, with both conformation types formed at a nearly equal proportion (data not shown). In addition, the Zn(II) was primarily coordinated with only one H of two bonded cyclo-HY molecules independent of which class the dipeptide adopted, in contrast to the cyclo-HH+Zn(II) system where Zn(II) was coordinated with two H residues of opposing “Class 2” β-bridge bonded dipeptides.

The ordered β-bridge like conformations of cyclo-HH mostly coexisted with relatively amorphous structures of cyclo-HH arranged in larger clusters (data not shown), with the ordered conformations positioned in the core and the amorphous layers partially surrounding in the perimeter, reminiscent of pseudo “core/shell” architectures. According to additional calculations, the outer amorphous layers can provide a stabilizing environment for the core ordered structures, thereby improving their durability. This type of architecture can stabilize the dots, thus enhancing the QY and the photoluminescence intensity.

Within the clusters of cyclo-HH+Zn(II), the ratio of Zn(II) to cyclo-HH dipeptide was higher in the core (0.5-1.13) than in the shell layers (0.42-0.82).

The ratio of Zn(II) to cyclo-HH dipeptide is approximately 1:1 for two adjacent cyclo-HH molecules, while the ratio of Zn(II) to chloride ions is approximately 1:2, thus indicating that the Zn(II) involved in “Class 2” β-bridge like conformations is coordinated with two imidazole rings of cyclo-HH dipeptide pairs along with the two chloride ions. The Zn(II) to cyclo-HH ratio was lower for the clusters comprising more adjacent peptides, while keeping the ratio of Zn(II) to chloride ions consistent. Nevertheless, elongated β-bridge like conformations comprising a 1:1 ratio of Zn(II) to cyclo-HH could still be observed.

For comparison, the ratio of Zn(II): cyclo-HY was less than 1:3, notably lower than the 1:1 ratio observed for cyclo-HH+Zn(II). Additionally, the ratio of Zn(II):cyclo-HY did not differ in the core of the clusters versus the shell layers. These structural differences between cyclo-HH and cyclo-HY in the presence of Zn(II) suggest that the emission of the cyclo-dipeptide assemblies is influenced by their ability to coordinate with Zn(II). The presence of two H within cyclo-HH could allow for the Zn(II) ions to “lock” in elongated “Class 2” β-bridge like conformations, which enhanced the QY of the assemblies.

Additional statistical analysis further focused on the structural and energetic properties of cyclo-HH self-assembly in the presence of Zn(II), and the extracted self-assembly procedure is presented in FIG. 39B. Statistical analysis of all instances of cyclo-HH “Class 2” β-bridge like conformations, irrespective of whether the pair of cyclo-HH was within a larger cluster or not, showed that the Zn(II) ion first coordinated with the H of one cyclo-HH dipeptide (FIG. 39Bi). Then, the H of a second cyclo-HH monomer coordinated with the Zn(II) to form a dimer, at a statistical proportion of 47.2% (FIG. 39Bii). Following the coordination, the two cyclo-HH monomers began to form hydrogen bond interactions between their backbone atoms (FIG. 39Biii), at a statistical proportion of 49.2%, to finally form β-sheet bridge-like configurations at a statistical proportion of 83.1% (FIG. 39Biv). The relatively low instances of the first two stages (<50%) indicate that a large proportion of cyclo-HH monomer remained separated in the solution, consistent with the DLS results. This analysis suggests that Zn(II) was not coordinated with preformed peptide structures, but rather drove the formation of the “Class 2” β-bridge like conformations following attraction to the peptide monomers at the very early stage of the assembly process. The aforementioned analysis focused on the Zn(II) ion simultaneously shared by two H side-chains. However, during the simulations, an additional Zn(II) ion was commonly present within the “Class 2” β-bridge like conformations, such that it was coordinated with one of the remaining H side-chains of the two β-bridge bonded cyclo-HH dipeptides. This is indicated by the nearly 1:1 ratio of Zn(II) to cyclo-HH in the “Class 2” β-bridge like conformations comprising two adjacent peptides (not shown).

Free energy calculations were further performed to elucidate the driving forces leading to the formation of clusters in the cyclo-HH+Zn(II) system. These studies revealed that the doping of cyclo-HH association by Zn(II) is initially driven by a relatively small energetic penalty for the transfer of Zn(II) to a lower dielectric medium, counterbalanced by single or pairs of cyclo-HH monomers attracting and pulling Zn(II) from the solvent to the peptide-rich environment, thus comprising an “environment-switching” mechanism. This mechanism could be observed within MD simulations, with Zn(II) first coordinated with cyclo-HH monomers promoting their self-assembly into pseudo “core/sell” clusters. Data related to these studies can be found in Kai Tau et al., Adv. Funct. Mater. 2020, 1909614, which is incorporated by reference as if fully set forth herein.

Light Emitting Diode Using Peptides Assemblies as Phosphors:

By combining a mixture of dried cyclo-HH+Zn(II) dots (upon solvent evaporation) and polydimethylsiloxane with a 420 nm emissive InGaN chip, a prototypical light emitting diode (LED) device using peptide self-assemblies as phosphors was fabricated.

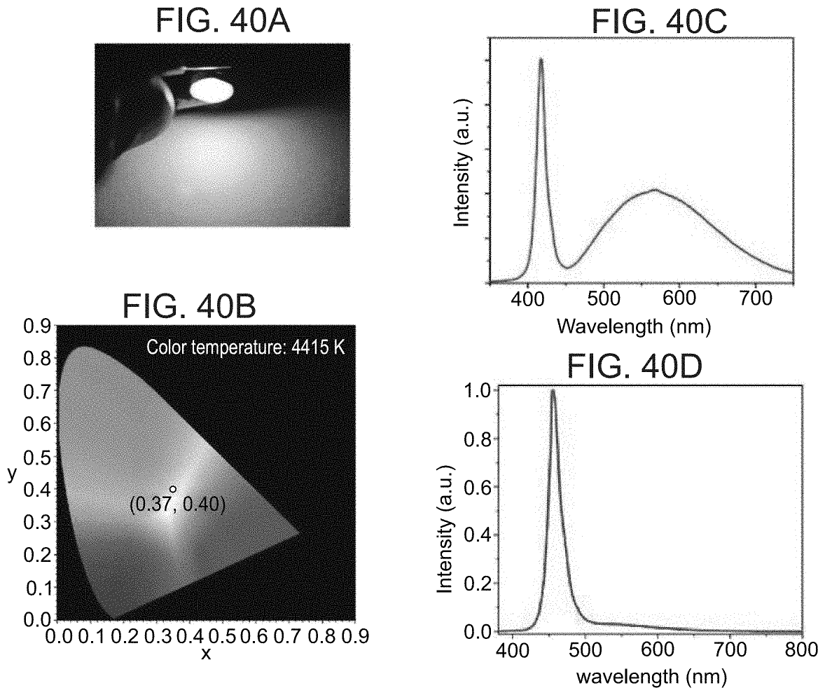

As shown in FIGS. 40A-C, bright green light with an emission around 565 nm was obtained when the LED operated under voltage of 3.0 V, with Commission Internationale de L′Eclairage (CIE) coordinates of (0.37, 0.40) and a color temperature of 4415 K. As a control, a LED comprising cyclo-HH alone showed the intrinsic blue emission of the chip (FIG. 40D), thus confirming the role of Zn(II) in enhancing green photoluminescence.

These results further demonstrate that the aromatic cyclodipeptide self-assembling photoactive structures show a promising prospect for use in the ecofriendly optoelectronic field, potentially bridging between the optical world and biological systems.

### Example 8

The assembly of cyclic(L-histidine-D-histidine) (denoted herein CHH) was studied. Highly fluorescent peptide dots, with a large quantum yield (>0.7), were constructed through “self-assembly locking strategy”. The CHH self-assemblies show bright fluorescence, allowing their use as an emissive layer in the photo- and electro-luminescent light emitting diodes (LEDs). A “self-encapsulation” strategy was used to construct a nanocarrier to effectively deliver anticancer drug into cancer cells with in situ monitoring. These studies show that bioinspired supramolecular functional components can be applied as novel multifunctional nanomaterials with unique features for optoelectronic or biological applications.

Materials and Methods:

Methods which are not described herein are as described in the Materials and Methods section hereinabove.

Materials: CHH was purchased from GL Biochem (Shanghai, China). Zinc nitrate (Zn(NO₃)₂), zinc chloride (ZnCl₂), zinc bromide (ZnBr₂), zinc iodide (ZnI₂), sodium nitrate (NaNO₃), polyvinyl pyrrolidone (PVP), 1-methyl-2-pyrrolidone (NMP), dimethylformamide (DMF), dimethyl sulfoxide (DMSO), isopropanol, ethanol were purchased from Sigma Aldrich (Rehovot, Israel). Poly(3,4-ethylenedioxythiophene): poly(styrenesulfonate) (PEDOT: PSS) (Clevios P VP AI 4083) was purchased from H. C. Stark. Poly (N-vinyl carbazole) (PVK) was purchased from Tokyo chemical industry Co. Ltd. 1,3,5-tris(N-phenylbenzimidazol-2-yl) benzene (TPBI) was purchased from Xi′an Polymer Light Technology Corp. PEO (M.W.=1000 000). Epirubicin hydrochloride (EPI) was purchased from Glentham life science, and DRAQ5 was purchased from Biolegend Inc. All materials were used as received without further purification. Water was processed using a Millipore purification system (Darmstadt, Germany) with minimum resistivity of 18.2 MΩ cm. 450 nm emissive InGaN chip (emission peak at 450 nm, operation under voltage of 3.0 V) was purchased from Greatshine Semiconductor Technology Co. Ltd.

Peptide self-assembly: Fresh stock solutions of CHH were prepared by dissolving the peptide into 5% (v/v) DMF/isopropanol at a concentration of 0.02 mmol mL-1. For assembly, 2.97 mg of metal salt Zn(NO₃)₂ was added into the peptide solution under vigorous sonication, and the colorless solution was incubated in an 80° C. water bath for 1 hour. The color of the solution subsequently turned into light yellow.

Crystal preparation: (1) CHH—ZnI₂: the weighed CHH powder was dissolved in 5% (v/v) DMF/isopropanol, to a concentration of 5.48 mg/mL, 6.38 mg of metal salt ZnI₂ was added under vigorous sonication, followed by a 1 hour incubation at 80° C. and filtration through a 0.45 μm PTFE membrane (Merck Millipore, Carrigtwohill, Ireland). Red rod crystals appeared after ten days and reached a maximum size after 30 days. (2) CHH—NaNO₃: the weighed CHH powder was dissolved into 5% (v/v) DMF/ethanol, to a concentration of 5.48 mg/mL, and 1.7 mg of metal salt NaNO₃ was added under vigorous sonication, followed by a 1 hour incubation at 80° C. and filtration through a 0.45 μm PTFE membrane (Merck Millipore, Carrigtwohill, Ireland). Colorless plaque-shaped crystals appeared after five days and reached a maximum size after 30 days. (3) CHH—Zn(NO₃)₂: the weighed CHH powder was dissolved in 5% (v/v) DMF/ethanol, to a concentration of 5.48 mg/mL, and 2.97 mg of metal salt Zn(NO₃)₂ was added under vigorous sonication, followed by a 1 hour incubation at 80° C. Yellow needle-shaped crystals appeared after 15 minutes and reached a maximum size within 2 hours.

Atomic force microscopy (AFM): 5 μL of sample solution was dropped onto a freshly cleaved mica surface and dried by N₂ purge (99.99%). The mica was then rinsed with water and gently purge dried with nitrogen. A topographic image was recorded under a Dimension icon AFM (Bruker) in the tapping mode at ambient temperature, with a 512×512-pixel resolution and a scanning speed of 1.0 Hz. Nanoscope Analysis software was used for data collection and analysis.

Mass spectrometry (MS): The CHH—Zn sample, along with the untreated CHH, were dissolved into a 1% (v/v) trifluoroacetic acid/water mixture. The MS experiment was performed using a LCMS Xevo-TQD system including an Acquity model UPLC and a triple quad mass spectrometer (Waters, Mass., USA). The positive electrospray ionization (ES+) channel was used for analysis.

Powder X-ray diffraction (PXRD): Self-assemblies of Cyclo-HH—Zn(NO₃)₂ was centrifuged at 15000 rpm for 20 minutes and the precipitates were washed three times with Milli-Q water, flash-frozen immediately, and lyophilized for 72 hours. The resulting powder samples were deposited on a quartz zero-background sample holder. The diffraction patterns were collected using a D8 ADVANCE diffractometer (Bruker, Germany) equipped with a linear detector LYNXEYE XE. Data collection was performed at room temperature with a scan range 2θ of 5-45°.

For cyclo-dipeptide-Zn structures, aliquots (10 μL) of cyclo-dipeptide-Zn were added into a glow discharge copper grid (400 mesh) coated with a thin carbon film for 2 minutes. Excess solution was then removed, and the grid was washed three times with deionized water. TEM images were viewed using a FEI Tecnai F20 electron microscope operating at 80 kV.

X-ray crystallography: Crystals suitable for diffraction were coated with Paratone oil (Hampton Research), mounted on loops and flash frozen in liquid nitrogen. Single crystal X-ray diffraction data measurement was performed using a Rigaku XtaLabPro system with CuKα1 (λ=1.5418 Å) radiation at 100(2) K. Data were collected and processed using CrysAlisPro 1.171.39.22a (Rigaku OD, 2015). The structure was solved by direct methods using SHELXT-2016/4 and refined by fullmatrix least squares against F2 with SHELXL-2013.

Fluorescence lifetime microscopy (FLIM): Fluorescence lifetime imaging was acquired using an LSM 7 MP two-photon microscope (Carl Zeiss, Weimar, Germany) coupled to the Becker and Hickl (BH) simple-Tau-152 system. Chameleon Ti: Sapphire laser system with 80 MHz repetition rate was used to excite the sample at 900 nm. Images were acquired using a Zeiss 20×1 NA waterimmersion objective. A Zeiss dichroic mirror (LP 760) was used to separate the excitation and emission light wavelengths. Emission light was collected via a hybrid GaAsP detector (HPM-100-40, BH, Berlin, Germany) with a GFP bandpass filter. The image acquisition time was 60 seconds in order to collect a sufficient number of photons.

Fluorescence spectroscopy and quantum yield (QY) measurement: 600 μL sample solution was pipetted into a 1.0 cm path-length quartz cuvette, and the spectrum was collected using a FluoroMax-4 Spectrofluorometer (Horiba Jobin Yvon, Kyoto, Japan) at ambient temperature. The excitation and emission wavelengths were set at 300-500 nm and 400-650 nm, respectively, with a slit of 2 nm. Absolute fluorescence QY measurements were performed using Quanta-Phi integrating sphere connected to Fluoromex-4.

Microfluidics experiments: Microfluidics experiments were performed as reported in Arnon et al., Nature Communications 2016, 7, 13190]. In a typical protocol, CycloHH—Zn(NO₃)₂ crystalline powder was inserted into the device. Then, a flow of fresh solutions was injected at a rate of 4 μl h-1 using Cetoni GmbH neMESYS Syringe Pumps (Korbussen, Germany) and glass HAMILTON syringes, 1,725 TLL of 250 μl. The process was examined under an Eclipse Ti-E inverted microscope (Nikon, Japan), equipped with a Zyla 4.2+sCMOS camera (Andor, UK), and images were captured at different time points.

Photoluminescence device fabrication and characterization: Commercially available InGaN chips were used at the bottom of the light emitting diode (LED) base. For preparation of the color conversion layer, CycloHH—Zn was blended into PVP at a mass ratio of 1:70, and the resulting mixtures were vacuum-dried at 60° C. for 30 minutes. The mixtures were applied on the InGaN chips and following curing at 80° C. for one hour, and the LEDs peptide phosphors were obtained.

Organic LED (OLED) device fabrication and characterization: ITO-coated glass substrates were cleaned ultrasonically in organic solvents (acetone and isopropyl alcohol), rinsed in deionized water, and then dried in an oven at 150° C. for 10 minutes. The substrates were cleaned by a UV-ozone treatment to enrich the ITO surface with oxygen, thereby increasing its work function. The approximately 30 nm thick PEDOT: PSS hole injection layer was spin-coated at 3000 rpm for 30 seconds on the ITO, followed by annealing in an oven at 150° C. for 15 minutes. Subsequently, the emissive layer of CycloHH—Zn blended into PVK was spin-coated at 3000 rpm for 35 seconds over the surface of the PEDOT:PSS film from the solution of NMP, followed by baking on a hot plate at 80° C. for 15 minutes to form the active region of the peptide-derived bio-OLED. Finally, the substrates were transferred to a vacuum chamber and a 30 nm thick TPBI electron transport layer was thermally deposited with base pressure of 3×10-4 Pa. Next, a 20 nm Ca and 100 nm thick Al cathode was deposited using a shadow mask 2 mm in width. The active area of the devices was thus 4 mm². The thermal deposition rates for TPBI and Ca/Al were 1,1, and 3 Å s⁻¹, respectively. The thickness of the films was measured using a Dektak XT (Bruker) surface profilometer and a spectroscopic ellipsometer (Suntech). The luminance-current-voltage (L-I-V) characteristics were measured using a computer-controlled Keithley 236 SMU and Keithley 200 multimeter coupled with a calibrated Si photodiode. Electroluminescence spectra were measured by an Ocean Optics 2000 spectrometer, which couples a linear charge-coupled device array detector ranging from 350 to 800 nm.

Live cells imaging using confocal microscopy (CLSM): HeLa cells were grown to 70-80% confluence in glass bottom cell culture dishes. Then, the cells were cultured with media containing the CycloHH—Zn+EPI at a concentration of 4 μg/mL for different durations. Next, the cells were stained using a DRAQ5TM dye diluted 1:1000 in PBS for 15 minutes at room temperature in the dark to allow staining of the nuclei. The cells were then washed twice with PBS. Imaging was performed using SP8 inverted confocal microscope (Leica Microsystems, Wetzlar, Germany). Excitation and emission ranges: λex=405 nm, λem=420-500 nm; EPI, λex=543 nm, λem=550-750 nm; DRAQ5, λex=633 nm, λem=750-780 nm.

FLIM analysis of cultured cells: HeLa cells seeded in dishes were treated with CycloHH—Zn+EPI at a concentration of 4 μg/mL for 30 minutes, 76 minutes, 125 minutes, 194 minutes, 270 minutes, and 420 minutes, followed by washing with PBS. The time-resolved fluorescence signal was acquired using an LSM 7 MP two-photon microscope (Carl Zeiss, Weimar, Germany) coupled to the Becker and Hickl (BH) simple-Tau-152 system. Images were acquired through a Zeiss 20 X/1 NA water-immersion objective. A Zeiss dichroic mirror (T690) was used to separate the excitation and the emission light.

An additional barrier filter was used to block emission light above 690 nm. Emission light was separated by a dichroic mirror (555 nm) and the two fluorescent lights were filtered by two bandpass filters (500-550 nm and 590-650 nm). Pseudocolored lifetime images were generated by assigning a color to the value of average fluorescence lifetime τm at each pixel. Emission light was collected via a hybrid GaAsP detector (HPM-100-40, BH, Berlin, Germany) with a Cherry bandpass filter.

Cyclic voltammetry: Electrochemical experiments were carried out using a CHI660A electrochemical workstation; Indium-tin-oxide (ITO) glass substrates (10 mm×50 mm×0.7 mm) served as a working electrode, Ag/AgCl as the reference electrode, and a Pt wire as the counter electrode. CHH—Zn were dissolved in dry dimethylformamide (DMF) with 0.1 M tetrabutylammonium hexafluorophosphate (TBAPF6) as the supporting electrolyte. All electrochemical measurements were performed in a nitrogen atmosphere.

Preparation of CHH—Zn+EPI: Fresh stock solutions of CHH were prepared by dissolving the peptide into 4% (v/v) DMSO/isopropanol at a concentration of 5.48 mg/mL. Subsequently, 0.25 mg EPI and 2.97 mg of metal salt Zn(NO₃)₂ were added under vigorous sonication, followed by incubation in an 80° C. water bath for 1 hour and overnight at room temperature. The obtained suspension was then centrifuged at 15000 rpm for 20 minutes and the precipitates were washed three times with Milli-Q water to remove any excess EPI and salts. To determine the loading capacity of EPI, the precipitated CHH—Zn+EPI nanoparticles were re-dissolved in DMSO and measured by UV-Vis absorption spectra with a range of known standard concentrations.

Release profile of CHH—Zn+EPI: CHH—Zn+EPI samples (2 mg/mL) were individually placed into 3.5 kDa dialysis tubes and dialyzed in 70 mL PBS buffer and acetate buffer at different pH values (pH 7.4 or 6.0). The dialysis was carried out by stirring inside an incubator shaker at 37° C. in the dark. Drug release was assumed to begin as soon as the dialysis chambers were placed into the buffer reservoirs. Aliquots (100 μL) of the solutions in the release reservoirs were removed for characterization at various time points. The concentration of released EPI was determined by measuring the absorption at 500 nm using a calibration curve prepared under the same conditions.

Determination of cytotoxicity: 2×105 cells/mL of HeLa cells were cultured in 96-well tissue microplates (100 μL per well) and allowed to adhere overnight at 37° C. CHH—Zn+EPI was added in cell growth medium at concentrations of 1, 2, and 4 m/mL. Half of each plate was seeded with cells, while the other half was used as a blank control. Medium with no CHH—Zn+EPI served as a negative control. After overnight incubation at 37° C., cell viability was evaluated using 3-(4,5-dimethylthiazolyl-2)-2,5-diphenyltetrazolium bromide MTT cell proliferation assay according to the manufacturer's instructions. Briefly, after overnight incubation at 37° C. with the CHH—Zn+EPI, 10 μL of 5 mg/mL MTT reagent dissolved in PBS was added to each of the 96 wells, followed by a 4 hours incubation at 37° C. Next, 100 μL extraction buffer (50% DMF, 20% SDS in Milli-Q water) was added to the wells, followed by 30 min incubation at 37° C. in the dark. Finally, color intensity was measured using an ELISA plate reader at 570 nm and background subtraction at 680 nm.

Live cells imaging using confocal microscopy (CLSM): HeLa cells were grown to 70-80% confluence in glass bottom cell culture dishes. Then, the cells were cultured with media containing the CHH—Zn+EPI at a concentration of 4 μg/mL for different durations. Next, the cells were stained using a DRAQ5TM dye diluted 1:1000 in PBS for 15 minutes at room temperature in the dark to allow staining of the nuclei. The cells were then washed twice with PBS. Imaging was performed using SP8 inverted confocal microscope (Leica Microsystems, Wetzlar, Germany). Excitation and emission ranges: λex=405 nm, λem=420-500 nm; EPI, λex=543 nm, λem=550-750 nm; DRAQ5, λex=633 nm, λem=750-780 nm.

FLIM analysis of cultured cells: HeLa cells seeded in dishes were treated with CHH—Zn+EPI at a concentration of 4 μg mL-1 for 30, 76, 125, 194, 270, and 420 minutes, followed by washing with PBS. The time-resolved fluorescence signal was acquired using an LSM 7 MP two-photon microscope (Carl Zeiss, Weimar, Germany) coupled to the Becker and Hickl (BH) simple-Tau-152 system. Images were acquired through a Zeiss 20 X/1 NA water-immersion objective. A Zeiss dichroic mirror (T690) was used to separate the excitation and the emission light. An additional barrier filter was used to block emission light above 690 nm. Emission light was separated by a dichroic mirror (555 nm) and the two fluorescent lights were filtered by two bandpass filters (500-550 nm and 590-650 nm). Pseudocolored lifetime images were generated by assigning a colour to the value of average fluorescence lifetime τm at each pixel. Emission light was collected via a hybrid GaAsP detector (HPM-100-40, BH, Berlin, Germany) with a Cherry bandpass filter.

Phasor analysis of FLIM data: The phasor-FLIM analysis was performed using the SPCImage 6.4 software. The fluorescence signal collected from each pixel of the image was transformed into Fourier space and a phasor map was constructed. From the FLIM measurements, the sine (S) and cosine (G) Fourier components of the lifetime decay were calculated for every pixel of the image, yielding the two phasor coordinates G and S, calculated using the following equations:

$${S}_{i , j ( ω )} = \frac{{\int }_{0}^{∞} I ( t ) sin ( n ω t ) dt}{{\int }_{0}^{∞} I ( t ) dt}$$

$${g}_{i , j ( ω )} = \frac{{\int }_{0}^{∞} I ( t ) cos ( n ω t ) dt}{{\int }_{0}^{∞} I ( t ) dt}$$

where the indices i and j represent the pixel of the image and I(t) represents the photon counts of the time bin, t, of the lifetime decay histogram of the corresponding pixel. ω=2πf, where f is the laser repetition frequency (i.e., 80 MHz in our experiments) and n is the harmonic frequency. Analysis of the phasor distribution was performed by cluster identification. In general, every possible lifetime can be mapped onto this universal representation of the decay (phasor plot), and multiple species are added vectorially. To obtain a single apparent lifetime for each sample, a cluster of pixel values were detected in specific regions of the phasor plot, and the average fluorescence lifetime τm was calculated from all pixels above a threshold of about 300 photons.

Results:

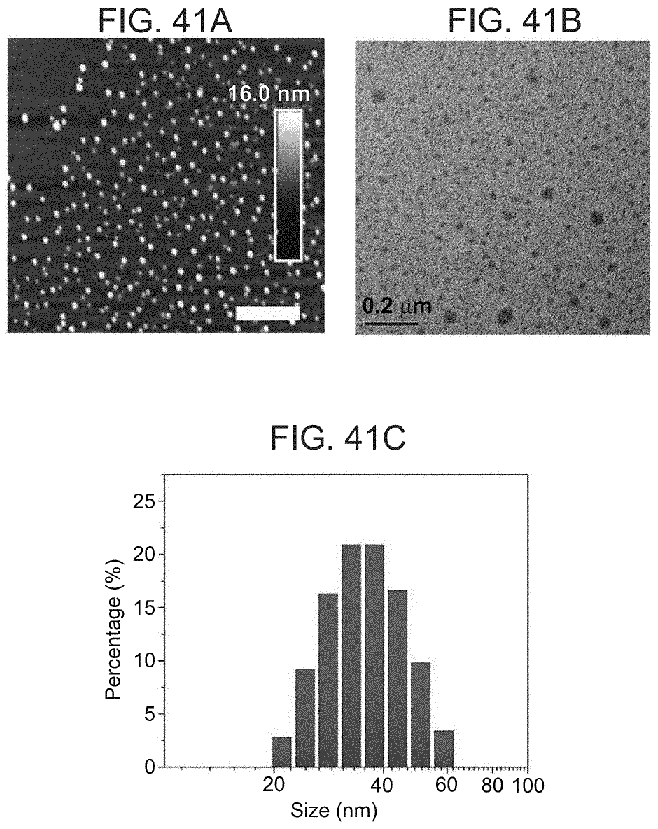

Co-assembly was performed by mixing CHH and Zn(NO₃)₂ (CHH—Zn) under controlled experimental conditions, resulting in nanostructure formation. Atomic force microscopy (AFM) and transmission electron microscopy (TEM) imaging confirmed the presence of nanoparticles with an average diameter of about 30 nm (FIGS. 41A-B), in agreement with dynamic light scattering (DLS) data (FIG. 41C).

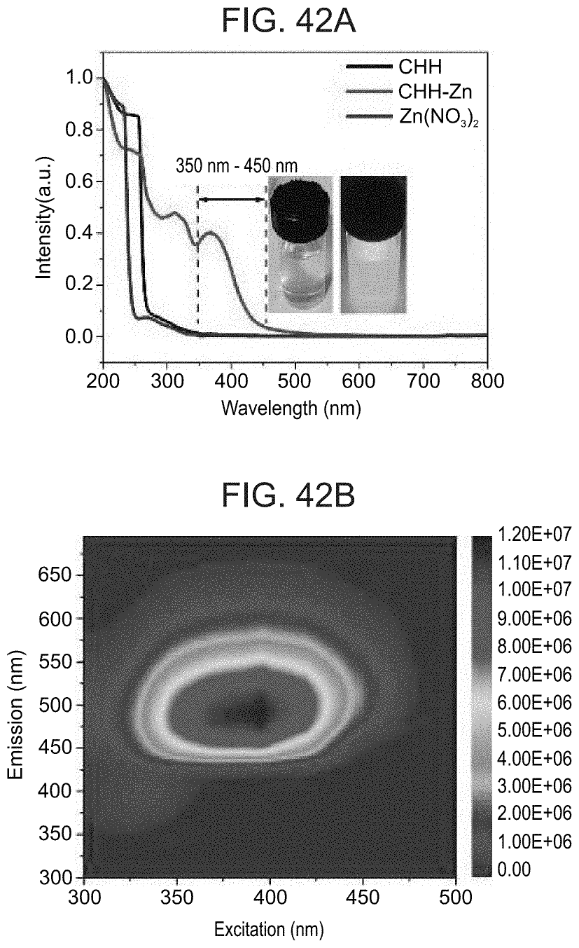

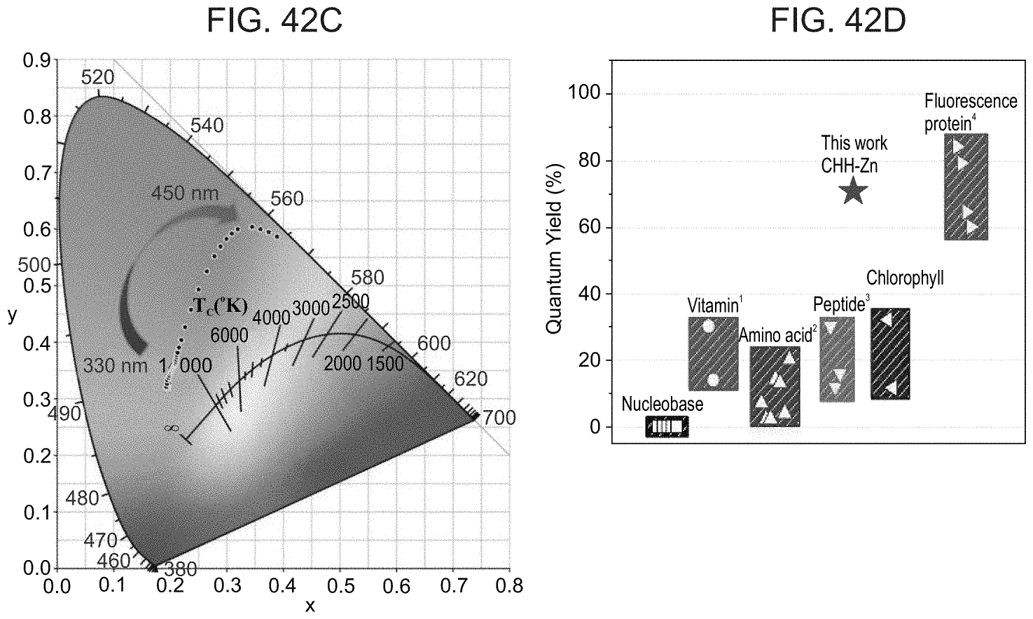

The optical properties of the CHH—Zn nanostructures are shown in FIGS. 42A-D. FIGS. 42A-B show the normalized UV-Vis absorption and excitation-emission matrix contour profiles of the CHH—Zn assemblies. By introducing Zn ions to the cyclic-dipeptide, an absorption peak extending between 350 nm to 450 nm was observed, indicating the formation of a Zn-related coordination structure. Upon excitation at 390 nm, the CHH—Zn peptide nanocrystals exhibited bright fluorescence emission centered at 500 nm. As shown in FIG. 42C, with the excitation wavelength changes from 330 to 450 nm, visible photoluminescence showed a pronounced red shift from cyan to green and variation of the central peak from 490 to 520 nm along with excellent linearity of the chromaticity coordinates. Such red-shift in the fluorescence emission spectra in response to a change in the excitation wavelength is termed as red edge excitation shift (REES). As shown in FIG. 42D, the maximum photoluminescence efficiency of the CHH—Zn self-assembly was about 70.6%, among the highest values reported so far for peptide-derived materials, and even comparable to inorganic quantum dots or GFP30-33.

Spectroscopic methods and control experiments were combined in order to obtain specific chemical and structural information. It was found that all Zn(II) present in the assembled system displayed a strong fluorescence signal and similar absorption and emission spectra, while comparative sodium nitrate-related self-assembly system and CHH showed similar absorption spectra and weak fluorescence emission intensity (data not shown). These findings suggest that the Zn(II)-peptide coordinated structure is formed by supplying Zn(II) to the cyclic-dipeptide system, which in turn determines the optical properties.

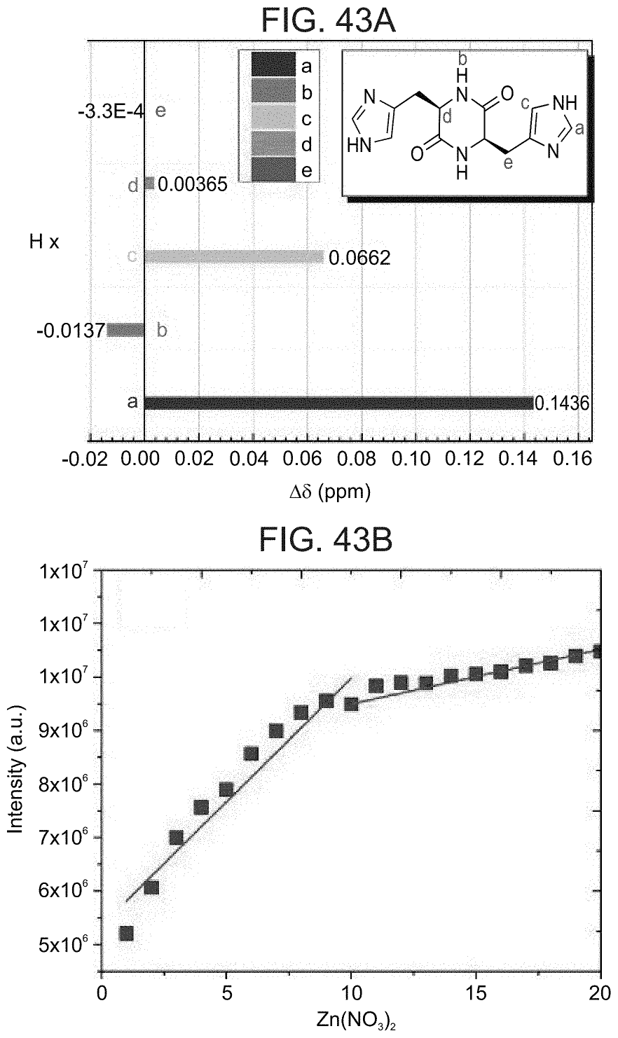

NMR analysis, presented in FIG. 43A showed that the imine protons of the side-chain imidazole ring downshifted Δδ=0.143 ppm (a position) after the addition of Zn(II), implying strong coordination through imine-imidazole nitrogen. The coordination of Zn ion and imidazole ring was further verified by mass spectrometry analysis of CHH with Zn(NO₃)₂, showing an m/z 611.2 band corresponding to the oligomer of [2MCHH+Zn²⁺] (data not shown).

Data obtained in these studies (see also FIG. 43B) suggest a specific self-assembly mechanism mediating CHH packing with nitrate.

To further characterize the self-assembly mechanism, both CHH—Zn(II) and CHH—NaNO₃ were crystallized and the resulting structures were analyzed via X-ray crystallography. The CHH—Zn(II) crystallizes in orthorhombic space group Pbcn, with one CHH molecule, one neutral [Zn(L)2I2] unit and one isopropanol molecule per asymmetric unit.

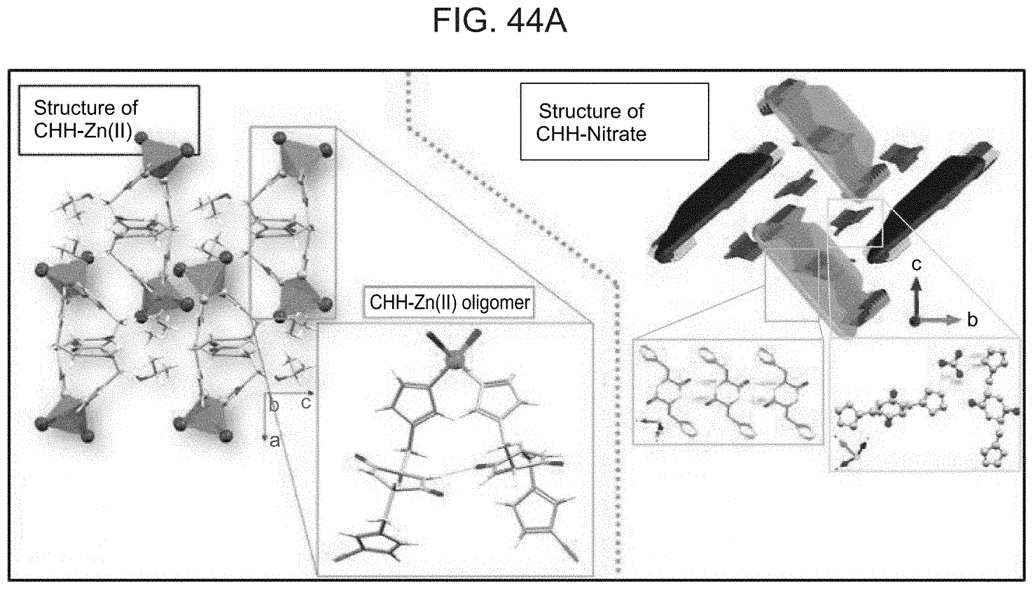

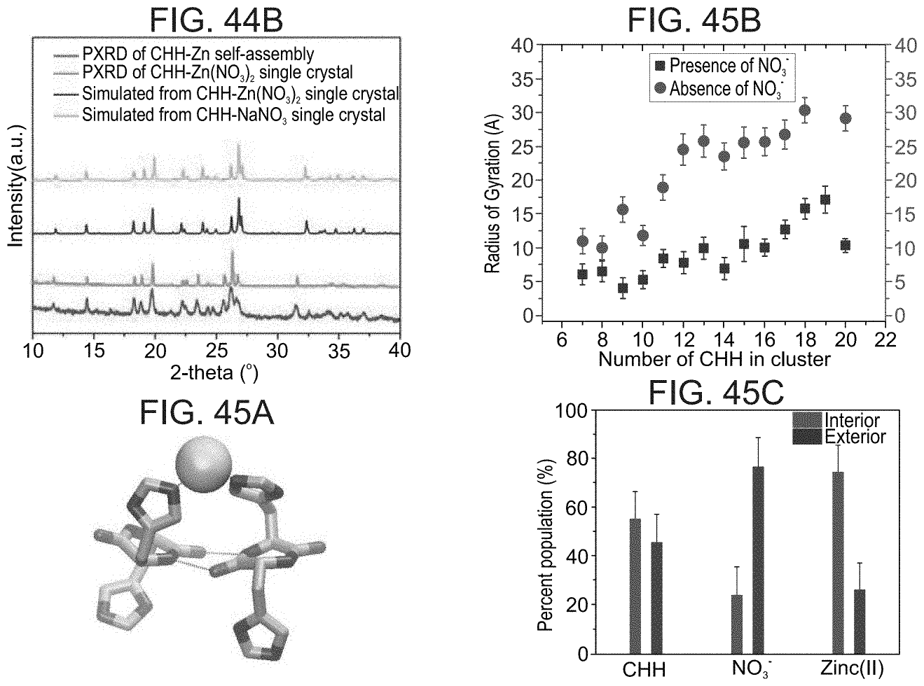

A perspective view of the Zn(II) center of the CHH—Zn(II) compound is illustrated in FIG. 44A (left) with a unit cell scheme. Each Zn(II) atom was coordinated with two ligands and two N-donor atoms from the imidazole groups of two different CHH molecules, occupying the apical coordination sites to generate a Zn(II) centered geometric tetrahedron. In turn, two adjacent cyclic-dipeptides were connected through a β-bridge like hydrogen bonding on the opposite sides of the backbone.

The X-ray determined structure of CHH—NaNO₃, shown in FIG. 44A (right) revealed a unique packing of the cyclic-dipeptides crystal in the monoclinic space group P21/c with two CHH and four nitrates in the unit cell. The components assembled to form a 1D chain with a hydrogen bond (N—H . . . O═C) of 2.889 Å (donor . . . acceptor) via a parallel (β-sheet hydrogen bonding network. The adjacent chains formed the extended structure through hydrogen bonds between the imidazole ring and nitrate groups.

CHH—Zn(NO₃)₂ single crystals were further examined through crystallographic analysis. Upon using a microfluids technique it has been visually observed that the Zn(NO₃)₂ crystals are densely packed with a growth rate of 0.01 μm s⁻¹ along the a direction, ultimately forming a needle shape (not shown). The resulting powder X-ray diffraction (PXRD) pattern shown in FIG. 44B and unit cell parameters of the CHH—Zn self-assemblies, highly resembled those of the formed CHH:Zn(NO₃)₂ crystals, indicating a similar molecular organization.

Using extensive crystallographic and confocal fluorescence lifetime microscopy (FLIM) studies combined with computational molecular dynamics and free-energy analysis, further insights into the formation of the CHH—Zn(NO₃)₂ clusters were gained by performing free-energy analysis of the different pathways which may lead to their formation. Some of the data obtained in these studies is presented in FIGS. 34A-C.

A plausible self-assembly mechanism of CHH and Zn(NO₃)₂ is depicted in FIG. 45D. The self-assembly of the CHH and Zn(II) can be observed at initial oligomerization step. Following the coordination of Zn(II) with the two histidine side-chains and stabilization of the dimer, the CHH monomers begin to form hydrogen bond interactions between their backbone atoms, forming a one-dimensional chain via β-sheet bridge-like interactions and subsequently generating an extended network through the linkage of nitrates. As the CHH one-dimensional chain grows, the chelation of CHH and Zn(II) is limited. Finally, the CHH—Zn(II) oligomer clusters are encapsulated and incorporated into CHH—NO₃— nanoassemblies.

The capability of the tested assemblies to serve as an emissive material in photo- and electro-luminescent prototypes was tested.

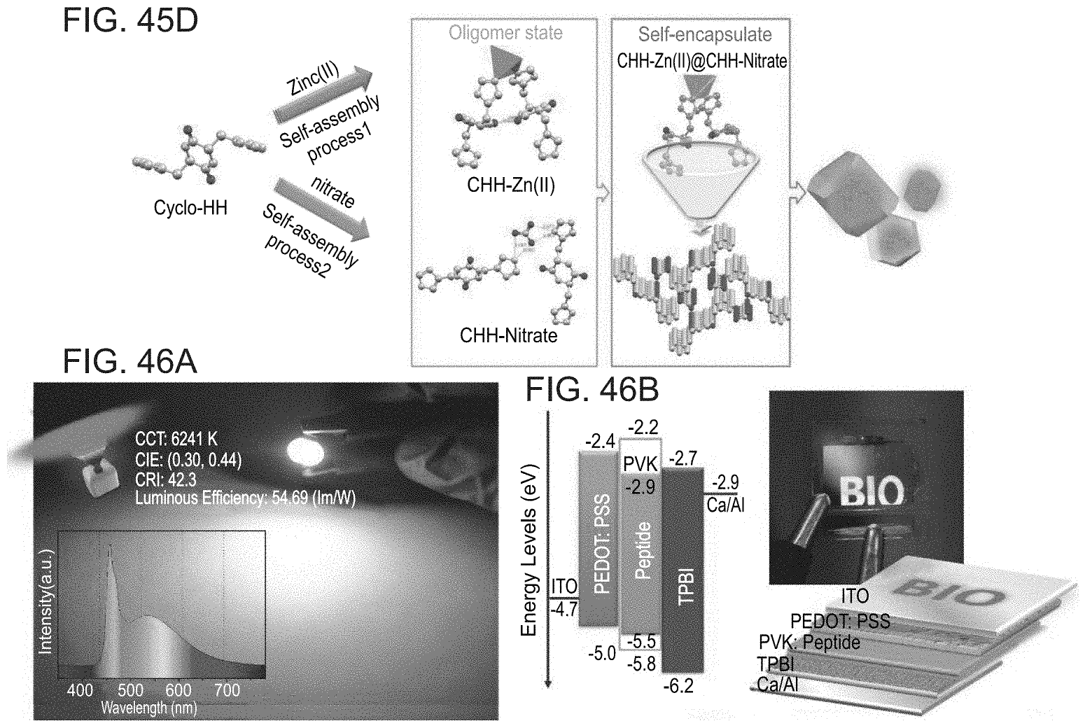

As shown in FIG. 46A, peptide-based phosphors were prepared by embedding CHH—Zn into polyvinyl pyrrolidone (PVP) at the mass ratio of 1:70. The peptide-based phosphor converted LED emitted bright green light with Commission Internationale de L′Eclairage (CIE) color coordinates of (0.31, 0.45) and achieved high luminous efficiency of 56.62 lm W⁻¹ at 20 mA drive current (not shown).

The CHH—Zn assembly was further utilized as a bio-organic light-emitting material in optoelectronics. A simple natural peptide derived bio-organic-LED (OLED) prototype was fabricated by using CHH—Zn-blended poly(N-vinyl carbazole) (PVK) as an emissive layer. As illustrated in FIG. 46B, the operation photographs present a close-up view of the bright, uniform, and defect-free surface green electroluminescence emission from the peptide-based OLED. The measured maximum luminance (Lmax) and current efficiency (ηc) reach as high as 1385 cd m-2 and 0.58 cd A-1, respectively, with a low applied Von of about 4 V. Due to the stable fluorescence, the bio-OLED showed no temporal degradation in the emission spectrum under the applied operating conditions, indicating significant potential for practical applications.

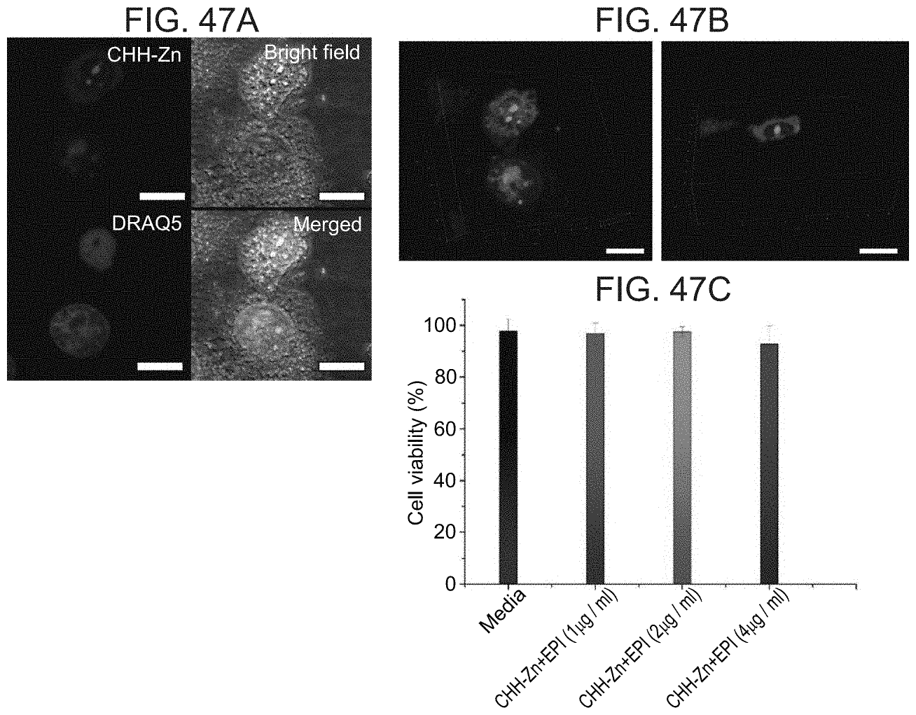

The self-assembled peptide nanoparticles were further tested for their use in bioimaging. High-resolution confocal fluorescence microscopy images of HeLa cells were collected following incubation with CHH—Zn and the DRAQ5 red DNA stain. As shown in FIG. 47A, the CHH—Zn structures were found to penetrate the cells and display bright green fluorescence under excitation of 405 nm. As shown in FIG. 47B, 3D imaging analysis indicated that CHH—Zn could effectively transport through the nuclear pore complex of HeLa cells and accumulate within the nucleolus region. As shown in FIG. 47C, in vitro cytotoxicity analysis demonstrated the excellent cytocompatibility of CHH—Zn peptide nanoparticles toward HeLa cells.

Based on the membrane permeability feature of the peptide structures, their potential applications for drug delivery was demonstrated.

The co-assembly of CHH—Zn and Epirubicin, an anthracycline drug used in chemotherapy, was experimentally confirmed through absorbance spectra, showing a 15.67% loading capacity of Epirubicin within the CHH—Zn nanoassemblies.

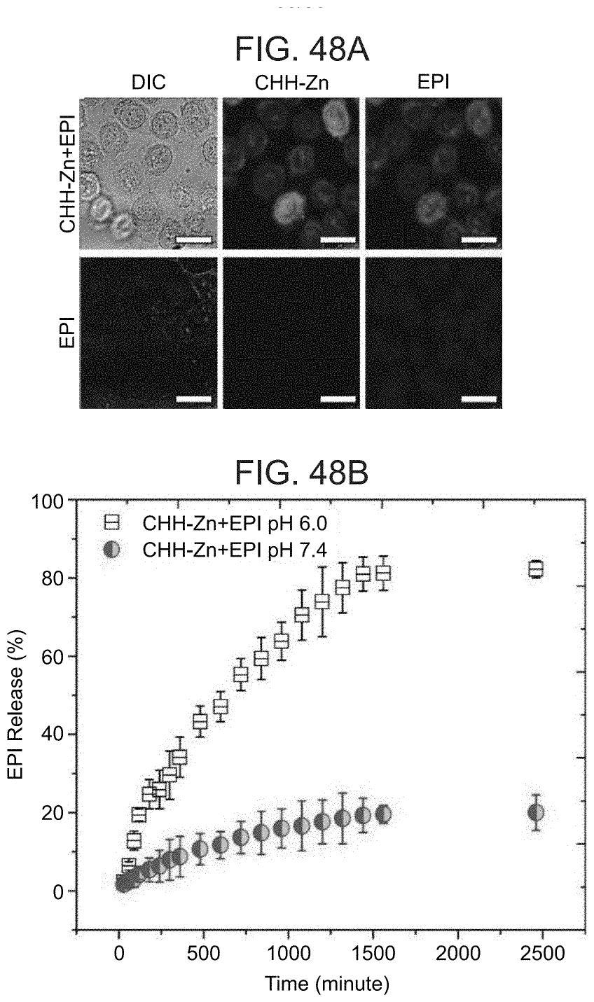

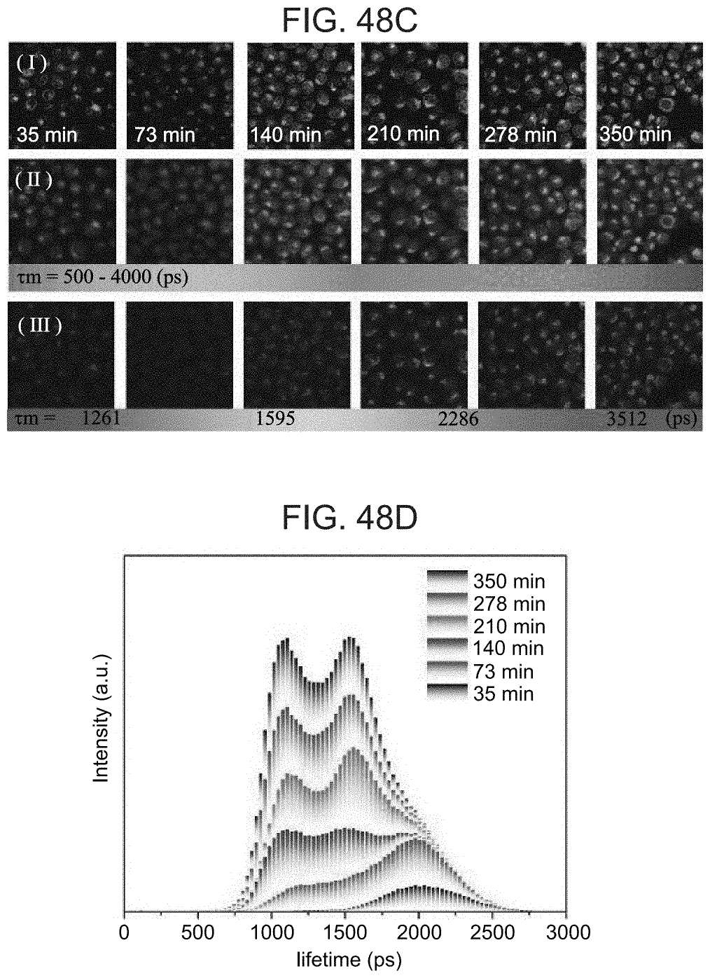

To examine the drug delivery potential of the newly-designed assemblies, HeLa cells incubated with CHH—Zn+Epirubicin or Epirubicin alone were examined by livecell confocal microscopy. As shown in FIG. 48A, the fluorescence intensity of intracellular Epirubicin in cells incubated with CHH—Zn+Epirubicin was significantly higher than that of Epirubicin alone, indicating efficient Epirubicin uptake and release into the nucleolus of HeLa cells via the CHH—Zn carrier. The Epirubicin release profiles shown in FIG. 48B suggested that the release of Epirubicin from the CHH—Zn can be efficiently triggered and accelerated by an acidic stimulus, which is favored for the acidic extracellular microenvironment of tumor tissues.

In order to further monitor the Epirubicin release process and eliminate autofluorescence from the biological system, the two-photon FLIM technique with phasor analysis was applied. Pixels with similar lifetimes are selected in the phasor diagram and the FLIM image is separated and painted into four subcellular compartments: cell membrane (about 3512 ps) cytoplasm (about 2286 ps), nucleus membrane (about 1595 ps), and nucleus (about 1261 ps) (not shown). After internalizing of Epirubicin into the cells, changes in its fluorescence lifetime can indicate changes in the subcellular microenvironment, reflecting drug release and transport. As shown in FIGS. 48C-D, with elongation of incubation time, more Epirubicin was released and, consequently, the fluorescence intensity of Epirubicin gradually increased, along with a decrease in the average lifetime.

These results indicate that the CHHZn+Epirubicin could accumulate around and bind to the cell membrane as early as 35 minutes of incubation with HeLa cells, and then be released in the cytoplasm due to the acidic environment and eventually accumulate in the nucleus. In addition, the release behavior of CHH—Zn+Epirubicin could be monitored by the variation of the fluorescent signal of CHH—Zn, showing that CHH—Zn not only promoted the transport of Epirubicin into HeLa cells, but also can be acted as a real-time optical monitor for the drug release process. These data demonstrate that the peptide nanostructures can be used to investigate the drug release in spatiotemporal mode and metabolism kinetics of cancer drugs in a certain organ or tissue.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

What is claimed is:

1. A light emitting system comprising a self-assembled structure formed of a plurality of cyclic peptides, at least a portion of said cyclic peptides being in association with metal ions, wherein each cyclic peptide in said plurality of cyclic peptides independently comprises from 2 to 6 amino acid residues, wherein at least two of said amino acid residues are each independently an aromatic amino acid residue, wherein said self-assembled plurality of cyclic peptides exhibits photoluminescence.

2. The light emitting system of claim 1, wherein in at least a portion, or all, of said plurality of cyclic peptides, each amino acid residue has the same chirality.

3. The light emitting system of claim 1, wherein in at least a portion, or all, of said plurality of cyclic peptides, at least one amino acid residue is an L-amino acid residue and at least one amino acid residue is a D-amino acid residue.

4. The light emitting system of claim 1, wherein in at least a portion, or all, of said plurality of cyclic peptides, each cyclic peptide is a cyclic dipeptide.

5. The light emitting system of claim 1, wherein in at least a portion, or all, of said plurality of cyclic peptides, each cyclic peptide is a cyclic homodipeptide.

6. The light emitting system of claim 5, wherein said cyclic homodipeptide comprises one L-amino acid residue and one D-amino acid residue.

7. The light emitting system of claim 1, wherein in at least a portion, or all, of said plurality of cyclic peptides, each cyclic peptide comprises at least one aromatic amino acid that comprises an imidazole in its side-chain.

8. The light emitting system of claim 7, wherein in at least a portion, or all, of said plurality of cyclic peptides, each cyclic peptide comprises at least two aromatic amino acid residues, each independently comprising said imidazole.

9. The light emitting system of claim 7, wherein in at least a portion, or all, of said plurality of cyclic peptides, each cyclic peptide is a cyclic homodipeptides which comprises two amino acid residues, each comprising said imidazole.

10. The light emitting system of claim 9, wherein said cyclic homodipeptide comprises one L-amino acid residue and one D-amino acid residue.

11. The light emitting system of claim 10, wherein each of said amino acid residues is a histidine residue.

12. The light emitting system of claim 1, wherein said association with said metal ions modulates at least one property of said photoluminescence of said self-assembled plurality of cyclic peptides.

13. The light emitting system of claim 12, wherein said association with said metal ions modulates an emission wavelength of said self-assembled plurality of cyclic peptides.

14. The light emitting system of claim 1, wherein said metal ions are multivalent metal ions.

15. The light emitting system of claim 1, wherein said self-assembled structure has an average size of less than 100 nm at least in one dimension or cross-section.

16. The light emitting system of claim 1, further comprising an excitation system configured to excite said self-assembled structure to emit light.

17. The light emitting system of claim 1, further comprising a therapeutically active agent in association with said self-assembled structure.

18. A pharmaceutical composition comprising the light emitting system of claim 1 and a pharmaceutically acceptable carrier.

19. A pharmaceutical composition comprising the light emitting system of claim 17 and a pharmaceutically acceptable carrier.

20. A method of treating a subject having a medical condition and/or for monitoring said medical condition and/or for monitoring said treating, the method comprising administering to the subject the light emitting system of claim 17, wherein said medical condition is treatable by said therapeutically active agent.
